SEQUENCE LISTING (61 sequences)

SEQ ID NO: 1 - DNA, 20 bp, Artificial Sequence
  Unique identifier nucleotide sequence for EGFR
acgcttaagaaaccgcctac

SEQ ID NO: 2 - DNA, 20 bp, Artificial Sequence
  Unique identifier nucleotide sequence for p53
cacagcacggaaacaggaga

SEQ ID NO: 3 - DNA, 20 bp, Artificial Sequence
  Unique identifier nucleotide sequence for erbB2
atatagaacgcccactcgca

SEQ ID NO: 4 - DNA, 20 bp, Artificial Sequence
  Unique identifier nucleotide sequence for erbB3
attatccaaaagcccgaccg

SEQ ID NO: 5 - DNA, 20 bp, Artificial Sequence
  Unique identifier nucleotide sequence for erbB4
tatatatgcgcgtgcaagcg

SEQ ID NO: 6 - DNA, 20 bp, Artificial Sequence
  Unique identifier nucleotide sequence for ER
agcttattgtttcggggtgc

SEQ ID NO: 7 - DNA, 20 bp, Artificial Sequence
  Unique identifier nucleotide sequence for pg8
atttttgtggcggatcgctg

SEQ ID NO: 8 - DNA, 20 bp, Artificial Sequence
  Unique identifier nucleotide sequence for ?-actin
acgtttatgacgtgttcggc

SEQ ID NO: 9 - DNA, 18 bp, Artificial Sequence
  Sequence for UP5 accessory region
taggcaggaagacaaaca

SEQ ID NO: 10 - DNA, 16 bp, Artificial Sequence
  Sequence for UP3 accessory region
acagcaccacagacca

SEQ ID NO: 11 - DNA, 21 bp, Artificial Sequence
  Sequence for TMP accessory region
ctgggctcaacccaggaagtg

SEQ ID NO: 12 - DNA, 6 bp, Artificial Sequence
  Sequence for Spacer A accessory region
aagctt

SEQ ID NO: 13 - DNA, 6 bp, Artificial Sequence
  Sequence for Spacer B accessory region
gcgcgc

SEQ ID NO: 14 - DNA, 6 bp, Artificial Sequence
  Sequence for Spacer C accessory region
cggccg

SEQ ID NO: 15 - DNA, 55 bp, Artificial Sequence
  Part of a oligonucleotide ID tag
taggcaggaagacaaacactgggctcaacccaggaagtgtggtctgtggtgctgt

SEQ ID NO: 16 - DNA, 18 bp, Artificial Sequence
  UP5 primer
taggcaggaagacaaaca

SEQ ID NO: 17 - DNA, 21 bp, Artificial Sequence
  TMP probe
cacttcctgggttgagcccag

SEQ ID NO: 18 - DNA, 20 bp, Artificial Sequence
  Primer for EGFR unique identifier nucleotide sequence
gtaggcggtttcttaagcgt

SEQ ID NO: 19 - DNA, 20 bp, Artificial Sequence
  Primer for p53 unique identifier nucleotide sequence
tctcctgtttccgtgctgtg

SEQ ID NO: 20 - DNA, 20 bp, Artificial Sequence
  Primer for erbB2 unique identifier nucleotide sequence
tgcgagtgggcgttctatat

SEQ ID NO: 21 - DNA, 20 bp, Artificial Sequence
  Primer for erbB3 unique identifier nucleotide sequence
cggtcgggcttttggataat

SEQ ID NO: 22 - DNA, 20 bp, Artificial Sequence
  Primer for erbB4 unique identifier nucleotide sequence
cgcttgcacgcgcatatata

SEQ ID NO: 23 - DNA, 20 bp, Artificial Sequence
  Primer for ER unique identifier nucleotide sequence
gcaccccgaaacaataagct

SEQ ID NO: 24 - DNA, 20 bp, Artificial Sequence
  Primer for pg8 unique identifier nucleotide sequence
cagcgatccgccacaaaaat

SEQ ID NO: 25 - DNA, 20 bp, Artificial Sequence
  Primer for ?-actin unique identifier nucleotide sequence
gccgaacacgtcataaacga

SEQ ID NO: 26 - DNA, 18 bp, Artificial Sequence
  Cy3UP5 primer
taggcaggaagacaaaca

SEQ ID NO: 27 - DNA, 18 bp, Artificial Sequence
  Cy5UP5 primer
taggcaggaagacaaaca

SEQ ID NO: 28 - DNA, 16 bp, Artificial Sequence
  UP3 primer
acagcaccacagacca

SEQ ID NO: 29 - DNA, 19 bp, Artificial Sequence
  ID sequence for aFGF
cattacccctaagggatgc

SEQ ID NO: 30 - DNA, 20 bp, Artificial Sequence
  ID sequence for bFGF
aattgcacaagagcccactc

SEQ ID NO: 31 - DNA, 20 bp, Artificial Sequence
  ID sequence for angiogenin
tacacgactttcgagcgcat

SEQ ID NO: 32 - DNA, 20 bp, Artificial Sequence
  ID sequence for TGF-alpha
aagaagcgacaacggaggaa

SEQ ID NO: 33 - DNA, 20 bp, Artificial Sequence
  ID sequence for TGF-betta
actacacgtacaccgagaga

SEQ ID NO: 34 - DNA, 20 bp, Artificial Sequence
  ID sequence for pg8
atttttgtggcggatcgctg

SEQ ID NO: 35 - DNA, 16 bp, Artificial Sequence
  UP3 primer
acagcaccacagacca

SEQ ID NO: 36 - DNA, 18 bp, Artificial Sequence
  UP5 primer
taggcaggaagacaaaca

SEQ ID NO: 37 - DNA, 20 bp, Artificial Sequence
  Unique identifier nucleotide sequence for ERK1/2
atctgagcaaacgcagcatg

SEQ ID NO: 38 - DNA, 20 bp, Artificial Sequence
  Unique identifier nucleotide sequence for p38 MARK
attatccaaaagcccgaccg

SEQ ID NO: 39 - DNA, 20 bp, Artificial Sequence
  Unique identifier nucleotide sequence for SARK/JNK
tttccgacatctgagccaac

SEQ ID NO: 40 - DNA, 20 bp, Artificial Sequence
  Unique identifier nucleotide sequence for I6B
ctaaaccctcatagggacac

SEQ ID NO: 41 - DNA, 20 bp, Artificial Sequence
  Unique identifier nucleotide sequence for GSK-3
tcatcacgactaccgatgca

SEQ ID NO: 42 - DNA, 20 bp, Artificial Sequence
  Unique identifier nucleotide sequence for Pg8
atttttgtggcggatcgctg

SEQ ID NO: 43 - DNA, 19 bp, Artificial sequence
  Unique identifier nucleotide sequence for caldesmon
gattcacgtgatccgatgc

SEQ ID NO: 44 - DNA, 20 bp, Artificial Sequence
  Unique identifier nucleotide sequence for adducin
tgatccgatagacgactgca

SEQ ID NO: 45 - DNA, 20 bp, Artificial Sequence
  Unique identifier nucleotide sequence for MARCK3
aaacgcagaagaggcacaca

SEQ ID NO: 46 - DNA, 20 bp, Artificial Sequence
  Unique identifier nucleotide sequence for NAP22/CAP23
tttggagcgtaagcagcatg

SEQ ID NO: 47 - DNA, 20 bp, Artificial Sequence
  Unique identifier nucleotide sequence for nNOS
gtaagcagagtaggcaacag

SEQ ID NO: 48 - DNA, 20 bp, Artificial Sequence
  Unique identifier nucleotide sequence for mGluR7A
agtaggcaatgtacccagac

SEQ ID NO: 49 - DNA, 20 bp, Artificial Sequence
  Unique identifier nucleotide sequence for calpastatin
aaagaccctcatgcagcaga

SEQ ID NO: 50 - DNA, 20 bp, Artificial Sequence
  Unique identifier nucleotide sequence for calpontin
aagaggcaacaaagcgaagg

SEQ ID NO: 51 - DNA, 20 bp, Artificial Sequence
  Unique identifier nucleotide sequence for neurogranin
actaagcctttcggagtctg

SEQ ID NO: 52 - DNA, 20 bp, Artificial Sequence
  Unique identifier nucleotide sequence for twitchin kinase
aacaaggctcatccgatgtc

SEQ ID NO: 53 - DNA, 20 bp, Artificial Sequence
  Unique identifier nucleotide sequence for titin kinase
acaaagcctcatccgaaagc

SEQ ID NO: 54 - DNA, 20 bp, Artificial sequence
  Unique identifier nucleotide sequence for myosin light chain kinase
cataaggcaaacagcgttgc

SEQ ID NO: 55 - DNA, 20 bp, Artificial Sequence
  Unique identifier nucleotide sequence for Pg8
atttttgtggcggatcgctg

SEQ ID NO: 56 - DNA, 18 bp, Artificial Sequence
  Forward primer
gacacagaggaggttttc

SEQ ID NO: 57 - DNA, 20 bp, Artificial Sequence
  Reverse primer
agtactcataggcattctct

SEQ ID NO: 58 - DNA, 20 bp, Artificial Sequence
  Forward primer
actctggagagtcaagatac

SEQ ID NO: 59 - DNA, 20 bp, Artificial Sequence
  Reverse primer
agaggacagactcactttat

SEQ ID NO: 60 - DNA, 18 bp, Artificial Sequence
  Forward primer (TAF1)
cgccagtgtgctggaatt

SEQ ID NO: 61 - DNA, 20 bp, Artificial Sequence
  Reverse primer (TAR)
cagtgtgatggatatctgca

(19) United States
(12) Patent Application Publication
(10) Pub. No.: US 2007/0003950 A1
(43) Pub. Date: Jan. 4, 2007

(54) Detecting targets by unique identifier nucleotide tags

(75) Inventors: Li Shen, Potomac, MD (US); Xiang Yu, Germantown, MD (US); Hui Cen, Oakland, CA (US)

Correspondence Address: MORRISON & FOERSTER LLP, 12531 HIGH BLUFF DRIVE, SUITE 100, SAN DIEGO, CA, 92130-2040, US

(73) Assignee: Superarray Bioscience Corporation, Frederick, MD (US)

(21) Appl. No.: 11/384,133

(22) Filed: Mar. 17, 2006

Related U.S. Application Data
(63) Continuation of application No. 10/269,790, filed on Oct. 10, 2002, now abandoned.
(60) Provisional application No. 60/327,763, filed on Oct. 10, 2001.

(51) Int. Cl. C40B 30/06; C12Q 1/68

(52) U.S. Cl. 435/6

(57) ABSTRACT

This invention relates generally to the field of target detection. In particular, the present inventions provides for methods and compositions for assaying a plurality of different non-nucleic acid targets or for assaying activities of a plurality of enzymes using, inter alia, oligonucleotide identification (ID) tags.

### CROSS-REFERENCE TO RELATED APPLICATIONS

The present application claims priority benefit of the provisional U.S. Patent Application Ser. No. 60/327,763, filed Oct. 10, 2001, the content of which is herein incorporated by reference in its entirety.

### BACKGROUND OF THE INVENTION

Many molecular events resulting in changes in protein expression and post-translational modification are implicated in the processes of growth, development, aging and disease. Synchronous detection of the molecular events is important in revealing the mechanism involved in these processes. The information obtained from detailed analysis of the molecular events occurring and contributing to these processes is valuable for the understanding of the underlying mechanisms of diseases and for the development of early detection, diagnosis, prognosis and therapy of human diseases.

For detection of nucleic acid targets, traditional methodology allows detection of a single target at a time. However, recent advancement of nucleic acid array technology has made it possible to simultaneously analyze thousands of nucleic acid targets in one sample by a single assay. The detection is achieved by immobilizing multiple nucleic acid targets at different locations on a support surface through base pairing between nucleic acid targets and their complementary strands affixed on the support surface. The multiple nucleic acid targets are detected by the specific base pairing of nucleotide, and the quantity of the nucleic acid targets are measured by the intensity of fluorescent signals that labeled on the bound target.

Simultaneous detection of protein targets has been more difficult to achieve. Specific binding between antibody and antigen has been widely employed to identify protein targets. Based on the same principle of nucleic acid array, many attempts have been made to produce a protein array by immobilizing proteins, such as antibodies or antigens, on a support surface at predetermined locations. Through the specific binding between proteins, particularly antibodies and antigens, multiple protein targets can be analyzed simultaneously. However, unlike nucleic acids, the activity of proteins is dependent on their 3-dimensional conformation. Usually, the 3-D structure of the water-soluble protein is preserved in aqueous liquid environment, while many hydrophobic proteins require lipid membrane or detergent to retain their biological activities. When the proteins are immobilization on a solid surface, proteins tend to denature at solid-liquid and liquid-air interfaces. Thus, antibody-based protein arrays by immobilizing the proteins on solid surfaces are largely unsuccessful due to loss or gradual loss of the antibody activity.

Color-coded beads in liquid suspension have been developed to encode antibodies in detection of multiple protein targets (Kruse, N., Pette, M., Toyka, K., and Rieckmann, P., J Immunol Methods. 1997, 210(2):195-203, 1997, Gordon R F and McDade R L., Clin Chem. 1997, 43(9):1799-801 1997.). The number of color-codes that can be employed in encoding antibodies is however limited by the detection system that is used adjunctively with a flow cytometer in sorting color-coded beads. The encoding and decoding system used in these methods limit the number of targets that can be detectable simultaneously in a single assay.

Oligonucleotide tags have been employed for tracking, retrieving and identifying nucleic acid target in vitro and in vivo. The most important benefit of using oligonucleotide tags for tracking targets is that very large number of unique oligonucleotide sequences can be produced to tag very large number of targets. U.S. Pat. No. 5,635,400 and U.S. Pat. No. 5,654,413 disclose methods for sorting polynucleotides onto surfaces of solid phase materials by the specific hybridization of oligonucleotide tags with their complements. By using about one million unique oligonucleotide tags to tag a total cDNA population, the tagged cDNA library is sorted by microbeads each immobilized with an oligonucleotide complementary to an oligonucleotide tag attached to the cDNAs. Therefore, the whole cDNA library can be separated into I million microbeads and analyzed simultaneously (Lynx, Calif.). As described in Shoemaker et al, Nature Genetics 14 (4): 450-465 (1996), a method has been developed to insert the oligonucleotide tags into the yeast genome and track deletion mutations in the yeast population to analyze the biological function of thousand of genes in parallel.

Oligonucleotide tags have been utilized to achieve highly sensitive immunoassays. As published in many references, such as U.S. Pat. No. 6,110,687, Sano et al., Science, 258:120, 1992; Case et al., J. Immunol Method, 223:93, 1999; Niemeyer et al., Nucleic Acid Res. 27:4553, 1999; Hendrickson et al., Nucleic Acid Res. 23:522, 1995; Schweitzer et al., Proc. Natl. Acad. Sci. USA 97:10113, 2000; Zhang et al. Proc. Natl. Acad. Sci. USA 98:5497, 2001, etc. Immuno-PCR is performed by using DNA molecules as template for amplification. However, in these contexts oligonucleotide tags are only used for amplification purposes, and only a single protein was detected at a time.

Hendrickson et al. (Nucleic Acid Res. 23:522, 1995, and U.S. Pat. No. 5,985,548) disclosed a method for amplification and simultaneous detection of a non-nucleic acid analyte (i.e., proteins) in fluid, in which the amplification was achieved by replicating a target nucleic acid sequence that has been co-immobilized with the analyte. These references also describe using the variation in length of nucleotide sequence as code to measure protein targets. The different lengths of nucleotide sequences are visualized by gel electrophoresis, and the length of a nucleotide sequence serves as the identity of the corresponding protein target. However, the use of the length of nucleotide sequences as codes in tracking, retrieving and identifing a large number of targets is limited by the fact that the size of nucleotide sequence may significantly affect conjugating nucleotide sequence to a reporter molecule, e.g., antibody. The differences in the length of nucleotide sequences often result in differences in conjugation efficiencies, and subsequently different reporting efficiency for different target. In addition, the different lengths of nucleotide sequences can affect their amplification efficiency by PCR. This method is limited by the potential discrimination in quantification of different lengths of nucleotide sequences, and the lack of a conventional method to analyze large number of different length nucleotide sequences also limited its application.

### BRIEF SUMMARY OF THE INVENTION

The present invention provides for methods and compositions for assaying a plurality of different non-nucleic acid targets or for assaying activities of a plurality of enzymes, using, inter alia, oligonucleotide identification (ID) tags.

A. Methods and Compositions for Assaying a Plurality of Different Non-Nucleic Acid Targets Using Tagged Reporter Ligands

In one aspect, the present invention is directed to a method for assaying a plurality of different non-nucleic acid targets in a sample, which method comprises: a) providing a plurality of reporter ligands, each said reporter ligand comprising a portion that specifically binds to a target present or suspected being present in a sample and an oligonucleotide identification (ID) tag, wherein said oligonucleotide ID tags in said reporter ligands are distinguishable from each other based on an identifiable property other than the length of said oligonucleotide ID tags; b) contacting said sample with said plurality of reporter ligands provided in step a) under suitable conditions to allow binding between said targets, if present in said sample, to said plurality of reporter ligands; c) separating reporter ligands bound to said targets from unbound reporter ligands; and d) assessing the identity and/or quantity of targets in said sample by detecting and/or quantifying said oligonucleotide ID tags in said reporter ligands bound to said targets. Related compositions and kits are also provided.

The present methods can be used to assay any non-nucleic acid targets. In one specific embodiment, the non-nucleic acid targets to be assayed are associated with a cellular component. For example, such non-nucleic acid targets can be comprised in fixed cells or tissue sections or comprised in a cell surface or an insoluble cellular component. The reporter ligands bound to the cellular-component-associated-targets can be separated from the unbound reporter ligands by any suitable methods, e.g., a wash step. The separating step can further comprise other separation procedures such as precipitation, centrifugation, flow cytometry or affinity immobilization.

In another specific embodiment, both the non-nucleic acid targets and the plurality of reporter ligands are soluble and the targets and the reporter ligands are contacted in a fluid to form soluble targets-reporter-ligands complexes. The soluble targets-reporter-ligands complexes can be separated from the unbound reporter ligands by any suitable methods. For example, the soluble targets-reporter-ligands complexes can be separated from the unbound reporter ligands by a difference in their molecular masses. In another example, the soluble targets-reporter-ligands complexes can be separated from the unbound reporter ligands by chromatography, electrophoresis, centrifugation or filtration. In still another example, the soluble targets-reporter-ligands complexes can be separated from the unbound reporter ligands by selective immobilization of targets-reporter-ligands complexes to a support surface followed by a wash step. The wash step can also involve a precipitation or centrifugation procedure to remove wash fluid from immobilized targets-reporter-ligands complexes.

In still another specific embodiment, the non-nucleic acid targets are soluble and the soluble targets are non-specifically immobilized to a support surface before the targets are contacted with the plurality of reporter ligands. The reporter ligands bound to the surface via binding to the targets can be separated from the unbound reporter ligands by any suitable methods, e.g., a wash step. The wash step can also involve a precipitation or centrifugation procedure to remove wash fluid from immobilized targets-reporter-ligands complexes.

In yet another specific embodiment, the non-nucleic acid targets are soluble and the soluble targets are immobilized to a support surface via a specific interaction between the targets and the support surface before the targets are contacted with the plurality of reporter ligands. For example, the specific interaction between the targets and the support surface can be effected via a single capture reagent on the support surface that specifically binds with a common moiety or epitope shared by the targets. Alternatively, the specific interaction between the targets and the support surface can be effected via a plurality of capture reagents on the solid surface, each of the capture reagents specifically binding with a different target. The reporter ligands bound to the surface via binding to the targets can be separated from the unbound reporter ligands by any suitable methods, e.g., a wash step. The wash step can also involve a precipitation or centrifugation procedure to remove wash fluid from immobilized targets-reporter-ligands complexes.

Any suitable moiety or substance can be used as the target-binding portion of the reporter ligand. For example, the target-binding portion of the reporter ligand can be an antibody; an antigen (when assaying antibody as target); a naturally occurring or synthetic ligand and receptor pairs (exemplary ligands being a protein, a peptide, a carbohydrate and a lipid, exemplary receptors being a protein, etc.); a binding motif, e.g., calmoludlin binding motif, protein A/G binding motif, an artificial key and lock imprint, e.g., plastic or silicon imprints of a protein, a peptide, a lipid, a carbohydrate; a lectin,.; a nucleic acid derived from an in vitro evolution process, e.g., aptamers evolved to bind proteins. Other exemplary moiety or substance that can be used as the target-binding portion of the reporter ligand include naturally occurring molecules, synthetic molecules, peptides, polypeptides, proteins, natural peptides, natural polypeptides, natural proteins, modified forms of peptides, modified forms of polypeptides, modified forms of proteins, post-translationally modified peptides, post-translationally modified polypeptides, post-translationally modified proteins, nucleotides, polynucleotides, modified nucleotides, modified polynucleotides, post-transcriptionally modified nucleotides, post-transcriptionally modified polynucleotides, natural lipids, natural polylipids, modified lipids, modified polylipids, natural saccharides, natural polysaccharides, modified saccharides, modified polysaccharides, cells, cell lysates, a micro-organism, a virus, polymers, mixtures of polymers, polypeptides, glycoproteins, protein complexes comprising more than one protein, antigens, phosphorylated proteins, antibodies, antibody fragments, single chain antibodies, phage displayed antibodies, lectins, lipids, carbohydrates, small organic molecules, polymers, sugars, oxy sugars, deoxy sugars, phosphorylated oxy sugars, phosphorylated deoxy sugars, saccharides, monosaccharides, polysaccharides, whole cells, nucleic acids, ribonucleic acids, deoxyribonucleic acids, polynucleotides, methylated DNA, lipids, carbohydrates, polymers, mixtures of polymers, small organic molecules, amino acids, steroids, modified steroids, fatty acids, micro-organisms, bacterial organisms, viral organisms, bacterial proteins, viral proteins, secreted molecules, cell surface proteins, subcellular organelles, nuclear proteins, naturally occurring form thereof, synthetic form thereof, derivatives thereof, complexes thereof, combinations thereof, and metabolites of biological processes.

The present methods can be used to assay any non-nucleic acid targets. For example, the targets can be proteins, peptides, lipids, carbohydrates, cells, cellular organelles, viruses, molecules and fragments, aggregates or complexes thereof. Other exemplary targets include naturally occurring molecules, synthetic molecules, a domain, a motif, a moiety of a molecule, a complex of molecules, proteins, polypeptides, peptides, post-translationally modified proteins, post-translationally modified polypeptides, glycoproteins, protein complexes comprising more than one protein, antigens, phosphorylated proteins, antibodies, antibody fragments, single chain antibodies, phage displayed antibodies, lectins, lipids, carbohydrates, small organic molecules, polymers, sugars, oxy sugars, deoxy sugars, phosphorylated oxy sugars, phosphorylated deoxy sugars, polymers, mixtures of polymers, saccharides, monsaccha nucleotides, polynucleotides, methylated DNA, lipids, carbohydrates, small organic molecules, amino acids, steroids, modified steroids, fatty acids, micro-organisms, bacterial organisms, viral organisms, bacterial proteins, viral proteins, secreted molecules, cell surface proteins, subcellular organelles, nuclear proteins, naturally occurring form thereof, synthetic form thereof, derivatives thereof, combinations thereof, complexes thereof, and metabolites of biological processes.

The oligonucleotide ID tags used in the present methods can be in any suitable forms. For example, the oligonucleotide ID tag in the reporter ligand can be DNA, RNA or a combination or analog thereof. In another example, the oligonucleotide ID tag in the reporter ligand can be single-stranded or double-stranded.

The oligonucleotide ID tags in the plurality of reporter ligands can be identified from each other based on any suitable property other than the length of the oligonucleotide ID tags. For example, the oligonucleotide ID tags in the plurality of reporter ligands can be identified from each other based on a difference in their nucleotide sequences, e.g., a difference in nucleotide sequence order, a nucleotide substitution, a nucleotide addition or a nucleotide deletion. Preferably, the oligonucleotide ID tags in the plurality of reporter ligand have about the same melting temperature or about the same number of nucleotides or about the same G:C content.

Preferably, when used in hybridization analysis for detecting oligonucleotide ID tags, all the tags should have similar Tm. When used in sequencing concatemers for detecting ID tags, similar in length is preferable. Only ID sequence region need to have similar melting temperature. For short oligonucleotide ID tag (e.g., less than 50 nt in ID region), the difference in melting temperature is preferably less than 10° C. For longer ID tags, e.g., cDNA based ID tag, the difference in melting temperature can be larger, but usually not over 20° C.

The oligonucleotide ID tags in the reporter ligands can be detected and/or quantified without dissociating the oligonucleotide ID tags from the target-binding portion of the reporter ligands. Alternatively, the oligonucleotide ID tags in the reporter ligands can be detected and/or quantified after they are dissociated from the target-binding portion of the reporter ligands.

The oligonucleotide ID tags in the reporter ligands can be detected and/or quantified without amplifying the oligonucleotide ID tags. Alternatively, the oligonucleotide ID tags in the reporter ligands can be detected and/or quantified after amplifying the oligonucleotide ID tags. The oligonucleotide ID tags can be amplified by any suitable methods, e.g., a nucleic acid replication method. Such exemplary nucleic acid replication methods include polymerase chain reaction (PCR), asymmetric polymerase chain reaction (aPCR), unidirectional linear polymerase reaction (LPR), T7 polymerase reaction, rolling cycle amplification, ligase chain reaction (LCR) and strand-displacement amplification. Other nucleic acid amplification methods, e.g., transcription based methods, can also be used.

The oligonucleotide ID tags in the plurality of reporter ligands can be identified and/or quantified by hybridization analysis, e.g., hybridization analysis under low, middle or high stringency conditions, parallel quantitative polymerase chain reaction (PCR) analysis or nucleotide sequencing analysis. In one example, the hybridization analysis is effected by contacting the oligonucleotide ID tags or amplified copies of oligonucleotide ID tags in the plurality of reporter ligands bound with the targets with an array of complementary nucleic acids immobilized on a support. In another example, the parallel quantitative polymerase chain reaction (PCR) analysis is effected by performing PCR reaction using an array of primers complementary to an identification nucleotide sequence of the oligonucleotide ID tags. In still another example, the nucleotide sequencing analysis is effected by amplifying the oligonucleotide ID tags to form double-stranded tags, cleaving the double-stranded tags using a restrictive endonuclease to release the oligonucleotide ID tags, ligating the oligonucleotide ID tags to form concatemers, sequencing the concatemers, and calculating the frequency of each oligonucleotide ID tag in the concatemers.

In one specific embodiment, the present method can further comprise performing a control experiment by adding a known amount of a reference target to the test sample together with unknown targets and detecting an amount of oligonucleotide ID tag representing the reference target to calibrate detection of the unknown targets.

In another aspect, the present invention is directed to a composition for assaying a plurality of non-nucleic acid targets in a sample, which composition comprises a plurality of reporter ligands, each said reporter ligand comprising a portion that specifically binds to a target present or suspected being present in a sample and an oligonucleotide identification (ID) tag, wherein said oligonucleotide ID tags in said reporter ligands are distinguishable from each other based on an identifiable property other than the length of said oligonucleotide ID tags. Preferably, the oligonucleotide ID tags in the plurality of reporter ligands have about the same melting temperature or about the same number of nucleotides or about the same G:C content.

In still another aspect, the present invention is directed to a kit for assaying a plurality of non-nucleic acid targets in a sample, which kit comprises: a) the above composition; b) means for separating said reporter ligands bound to said targets from said unbound reporter ligands; and c) means for detecting and/or quantifying said oligonucleotide ID tags in the reporter ligands. The kit can further comprise an instruction for simultaneously assaying a plurality of non-nucleic acid targets in a sample.

In yet another aspect, the present invention is directed to a composition, which composition comprises a plurality of complexes formed between a plurality of different non-nucleic acid targets and a plurality of corresponding reporter ligands, each said reporter ligand comprising a portion that specifically binds to a target and an oligonucleotide identification (ID) tag, wherein said oligonucleotide ID tags in said reporter ligands are distinguishable from each other based on an identifiable property other than the length of said oligonucleotide ID tags. Preferably, the composition is substantially free of reporter ligands unbound to any targets.

The present methods, compositions and kits can be used in assaying any suitable number of non-nucleic acid targets, preferably simultaneously. For example, the present methods, compositions and kits can be used in assaying at least 2, 5, 10, 50, 100, 500, 1,000, 5,000, 10,000 or more non-nucleic acid targets. Preferably, the present methods, compositions and kits are used in assaying a group of structurally and/or functionally related non-nucleic acid targets, e.g., proteins.

B. Methods and Compositions for Assaying a Plurality of Different Non-Nucleic Acid Targets Using Tagged Antagonists

In yet another aspect, the present invention is directed to a method for assaying a plurality of different non-nucleic acid targets in a sample, which method comprises: a) providing a plurality of target antagonists, each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags; b) providing a plurality of receptor ligands, each receptor ligand specifically binding to a different target and its corresponding reporter antagonist in a competitive manner; c) contacting a sample with said plurality of target antagonists and said plurality of receptor ligands provided in steps a) and b) under suitable conditions to allow competitive binding between said targets, if present in said sample, and their corresponding reporter antagonists, to their corresponding receptor ligands; d) separating said target antagonists bound to said receptor ligands from said unbound target antagonists; and e) assessing the identity and/or quantity of targets in said sample by detecting and/or quantifying said oligonucleotide ID tags in target antagonists bound to said receptor ligands or said unbound target antagonists.

The sample can be contacted with the plurality of target antagonists and the plurality of receptor ligands in any suitable order. For example, the sample can be contacted with the plurality of target antagonists first and then contacted with the plurality of receptor ligands. Alternatively, the sample can be contacted with the plurality of target antagonists and the plurality of receptor ligands simultaneously. It is also possible that the sample be contacted with the plurality of receptor ligands first and then contacted the plurality of target antagonists.

In yet another aspect, the present invention is directed to a composition for assaying a plurality of non-nucleic acid targets in a sample, which composition comprises a plurality of target antagonists, each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags. The composition can further comprise a plurality of receptor ligands, each receptor ligand specifically binding to a different target and its corresponding reporter antagonist in a competitive manner.

In yet another aspect, the present invention is directed to a kit for assaying a plurality of non-nucleic acid targets in a sample, which kit comprises: a) the above composition; b) means for separating the target antagonists bound to the receptor ligands from the unbound target antagonists; and c) means for detecting and/or quantifying said oligonucleotide ID tags in the target antagonists bound to the receptor ligands or said unbound target antagonists. The kit can further comprise an instruction for simultaneously assaying a plurality of non-nucleic acid targets in a sample.

In yet another aspect, the present invention is directed to a composition, which composition comprises a plurality of complexes formed between a plurality of receptor ligands and their corresponding target antagonist, wherein each said receptor ligand specifically binds to a different target or its corresponding reporter antagonist in a competitive manner and each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags.

In yet another aspect, the present invention is directed to a method for assaying a plurality of different non-nucleic acid targets in a cell, which method comprises: a) providing a plurality of target antagonists, each said target antagonist comprising a portion that specifically associates with a corresponding cellular component and an oligonucleotide identification (ID) tag, wherein said oligonucleotide ID tags in said target antagonists are distinguishable from each other based on an identifiable property other than the length of said oligonucleotide ID tags; b) delivering, e.g., transfecting, said plurality of target antagonists into said cell to allow competitive interaction between said targets, if present in said cell, and said target antagonists, with said cellular components; c) obtaining an equal amount of said cellular components associated with said targets or said target antagonists; and d) assessing the identity and/or quantity of targets in said cell by detecting and/or quantifying said oligonucleotide ID tags in said target antagonists associated with said cellular components. Preferably, the equal amount of the cellular components associated with the targets or target antagonists is obtained by isolating a biological structure, e.g., the cytosol, a plasma membrane, nucleus, endoplasmic reticulum, mitochondria, Golgi complexes, cytoskeleton, and other cellular organelles from the cell.

The general teachings of the above Section A, e.g., properties or numbers regarding to the non-nucleic acid targets, the oligonucleotide ID tags, and various separation, amplification, hybridization and sequencing procedures, etc., are also applicable to this Section B.

C. Methods and Compositions for Assaying Activities of a Plurality of Enzymes Using Tagged Reporter Substrates

In yet another aspect, the present invention is directed to a method for assaying activities of a plurality of enzymes in a sample, which method comprises: a) providing a plurality of reporter substrates, each said reporter substrate comprising a portion that can be modified by a corresponding enzyme in a sample and an oligonucleotide identification (ID) tag, wherein said oligonucleotide ID tags in said reporter substrates are distinguishable from each other based on an identifiable property, e.g., length or an identifiable property other than the length, of said oligonucleotide ID tags; b) contacting said plurality of reporter substrates with said sample under suitable conditions to allow each enzyme, if present in said sample, to catalyze a modification reaction on its corresponding reporter substrate; c) separating modified reporter substrates from unmodified reporter substrates; and d) assessing the activities of said enzymes in said sample by detecting and/or quantifying said oligonucleotide ID tags in said modified reporter substrates. The enzymes to be assayed can exist in vivo, i.e., in cells or in a multi-cellular organism, or in vitro, i.e., in a cell free environment. The enzymatic activity can also be assayed in situ.

The modified reporter substrates can be separated from the unmodified reporter substrates by any suitable methods, e.g., by contacting the reporter substrates with a capture reagent that specifically binds to the modification portion of the reporter substrates and that is immobilized on a surface.

In yet another aspect, the present invention is directed to composition for assaying activities of a plurality of enzymes in a sample, which composition comprises a plurality of reporter substrates, each said reporter substrate comprising a portion that can be modified by a corresponding enzyme in a sample and an oligonucleotide identification (ID) tag, wherein said oligonucleotide ID tags in said reporter substrates are distinguishable from each other based on an identifiable property, e.g., length or an identifiable property other than the length of said oligonucleotide ID tags.

In yet another aspect, the present invention is directed to a kit for assaying activities of a plurality of enzymes in a sample, which kit comprises: a) the above composition; b) means for separating the modified reporter substrates from the unmodified reporter substrates; and c) means for detecting and/or quantifying said oligonucleotide ID tags in the modified reporter substrates. The kit can further comprise an instruction for simultaneously assaying a plurality of enzymes in a sample.

In yet another aspect, the present invention is directed to a composition, which composition comprises a plurality of reporter substrates, each said reporter substrate comprising a portion that has been modified by a corresponding enzyme in a sample and an oligonucleotide identification (ID) tag, wherein said oligonucleotide ID tags in said reporter substrates are distinguishable from each other based on an identifiable property, e.g., length or an identifiable property other than the length of said oligonucleotide ID tags.

The general teachings of the above Section A, e.g., properties and numbers regarding to the non-nucleic acid targets, the oligonucleotide ID tags, and various separation, amplification, hybridization and sequencing procedures, etc., are also applicable to this Section C.

### BRIEF DESCRIPTION OF THE SEVERAL VIEWS OF THE DRAWING(S)

FIG. 1 depicts detection for soluble targets using molecular weight based separation scheme to separate reporter ligand-target complexes from free reporter ligands.

FIG. 2 depicts detection for soluble targets using selective immobilization scheme to separate reporter ligand-target complexes from unbound reporter ligands.

FIG. 3 depicts detection of specific immobilized targets.

FIG. 4 depicts detection of non-specific immobilized targets (e.g., fixed cell or tissue section or non-specific immobilized soluble cell lysate).

FIG. 5 depicts detection of soluble targets in a competition assay.

FIG. 6 depicts detection of targets on a cell surface.

FIG. 7 depicts detection of enzyme activity in a living cell.

FIG. 8 depicts composition of an exemplary oligonucleotide ID tag.

### DETAILED DESCRIPTION OF THE INVENTION

For clarity of disclosure, and not by way of limitation, the detailed description of the invention is divided into the subsections that follow.

A. Definitions

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.”

As used herein, “target” refers to a substance or moiety to be detected or assayed by the methods and compositions of the present invention.

As used herein, “ligand” refers to a substance or moiety that comprises a portion capable of specific binding with a desired target.

As used herein, “oligonucleotide ID tag” refers to a nucleic acid substance with a defined nucleotide sequence(s).

As used herein, “reporter ligand” refers to a ligand coupled with an oligonucleotide ID tag and capable of specifically binding with a target.

As used herein, “reporter antagonist” refers to a substance or moiety coupled with an oligonucleotide ID tag and capable of competing with target for binding with a ligand or associating with a cellular component.

As used herein, “reporter substrate” refers to a substance or moiety coupled with an oligonucleotide ID tag and can be modified by an enzyme in an enzymatic reaction.

As used herein, “capture ligand” refers to a substance or moiety capable of specifically binding a target and has been immobilized by attachment to an appropriate support surface.

As used herein, “receptor ligand” refers to a substance or moiety capable of specifically binding with both a target and an antagonist in a competition assay.

As used herein, “support surface” refers to a surface material onto which, various substances, e.g., targets or target-ligand complexes, can be immobilized.

As used herein, “said oligonucleotide ID tags in said reporter ligands are distinguishable from each other based on an identifiable property other than the length of said oligonucleotide ID tags” means that the different oligonucleotide ID tags are distinguishable from each other based on any identifiable physical, chemical and/or biological property other than the length of the oligonucleotide ID tags. Such exemplary identifiable properties include the differences in the nucleotide sequences, sensitivity to nuclease, e.g., restriction enzyme, digestion, ability or inability to form a secondary structure, e.g., hairpin structure, compositions, e.g., containing DNA or RNA or other types of modifications, structures, e.g., being single-stranded, double stranded, triple-stranded, or being in A-, B- or Z-form, or encoded biological activities, e.g., promoter activities, or a combination thereof.

As used herein, “nucleic acid” refers to any nucleic acid containing molecule including, but not limited to DNA, RNA or PNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

As used herein, “sample” refers to anything which may contain a target (or an analyte) or an enzyme to be assayed using the present methods and/or compositions. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods, e.g., magnetically activated cell sorting, may be applied to separate or enrich target cells from a body fluid sample such as blood.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “fluid” refers to any composition that can flow. Fluids thus encompass compositions that are in the form of semi-solids, pastes, solutions, aqueous mixtures, gels, lotions, creams, and other such compositions.

As used herein the term “assessing” is intended to include quantitative and/or qualitative determination of a target or enzyme present in the sample, and also of obtaining an index, ratio, and percentage, visual or other value indicative of the level of the target in the sample. Assessment may be direct or indirect and the chemical species actually detected need not of course be the analyte itself but may for example be a derivative thereof or some further substance.

As used herein, “targets associated with a cellular component” refers to targets that, in whatever manner, are associated with an intact cell, a subcellular interior or structure, extracellular matrix, or intercellular junction. For example, the targets can be associated with a cellular membrane, e.g., a plasma membrane or a membrane of a subcellular organelle or structure, a cell wall or extracellular matrix. Alternatively, the targets can be enclosed in a cellular interior, e.g., cytosol or interior of a subcellular organelle.

As used herein, “melting temperature” (“Tm”) refers to temperature at which about 50% of a given oligonucleotide is hybridized to its complementary strand.

As used herein, “the oligonucleotide ID tags in the plurality of reporter ligand have about the same melting temperature” means that the melting temperature of different oligonucleotide ID tags are sufficiently close so that same annealing, denaturing or hybridization conditions can be used without affecting an assay result for its intended purpose. Preferably, the difference between the highest and lowest melting temperatures is less than 20° C., and preferably less than 10° C., 5° C. or 1° C.

As used herein, “the oligonucleotide ID tags in the plurality of reporter ligand have about the same number of nucleotides” means that the difference between the largest and smallest number of nucleotides is less than 500 nucleotides. Preferably, the difference between the largest and smallest number of nucleotides is less than 400, 300, 200, 100, 50, 40, 30, 20, 10, 9, 8, 7, 6, 5, 4, 3, 2, 1 or 0 nucleotide(s).

As used herein, “protein modification” refers to addition of a peptidic or non-peptidic moiety to a protein that cannot be considered as the elongation of the peptidic chain of the protein. The addition of the peptidic or non-peptidic moiety can be in vivo or in vitro. The peptidic or non-peptidic moiety can be added to a pure protein or a protein or peptidic component of a complex containing such protein or peptide. Preferably, “protein modification” refers to post-translational protein modification. Exemplary post-translational protein modification include phosphorylation, acetylation, methylation, ADP-ribosylation, addition of a polypeptide side chain, addition of a hydrophobic group, and addition of a carbohydrate.

As used herein, “antibody” refers to specific types of immunoglobulin, i.e., IgA, IgD, IgE, IgG, e.g., IgG₁, IgG₂, IgG₃, and IgG₄, and IgM. An antibody can exist in any suitable form and also encompass any suitable fragments or derivatives. Exemplary antibodies include a polyclonal antibody, a monoclonal antibody, a Fab fragment, a Fab′ fragment, a F(ab′)₂ fragment, a Fv fragment, a diabody, a single-chain antibody and a multi-specific antibody formed from antibody fragments.

As used herein: “stringency of hybridization” in determining percentage mismatch is as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures (See generally, Ausubel (Ed.) Current Protocols in Molecular Biology, 2.9A. Southern Blotting, 2.9B. Dot and Slot Blotting of DNA and 2.10. Hybridization Analysis of DNA Blots, John Wiley & Sons, Inc. (2000)).

As used herein, “a group of structurally and/or functionally related non-nucleic acid targets (e.g., proteins)” refers to a group of non-nucleic acid targets (e.g., proteins), at their natural status, that are structurally linked, located at the same cellular locations, e.g., cellular organelles, located in the same tissues or organs, expressed and/or be functional in the same biological stages, e.g., a particular cell cycle stage or developmental stage, or expressed and/or be functional in the same biological pathway, e.g., a particular metabolism pathway, signal transduction pathway, etc. The “group of structurally and/or functionally related non-nucleic acid targets (e.g., proteins)” need only include at least two non-nucleic acid targets (e.g., proteins) belonging to the same group. The “group of structurally and/or functionally related non-nucleic acid targets (e.g., proteins)” can preferably include more than two non-nucleic acid targets (e.g., proteins) belonging to the same group, e.g., a majority of or even all the non-nucleic acid targets (e.g., proteins) belonging to the same group.

B. Exemplary Embodiments

Methods and compositions are provided for analysis, preferably, simultaneous analysis, of multiple non-nucleic acid targets. In the subject methods and compositions, an oligonucleotide ID tag is attached to a ligand to form a reporter ligand, wherein the unique identifier sequence of the oligonucleotide ID tag is used as an identification code of the reporting molecule. The reporter ligand selectively binds to the target, and a number of separation schemes may be employed to separate target bound reporter ligand complex from unbound reporter ligand. The oligonucleotide ID tag associated with target-reporter ligand complex is then detected. The detection for an oligonucleotide ID tag is in response to the presence of a target, the unique identifier sequence of an oligonucleotide ID tag determines identify of the target, and the amount of an oligonucleotide ID tag determines amount of the target. Through amplification of an oligonucleotide ID tag by means of nucleic acid replication, the sensitive detection for a target is achieved. The subject methods and compositions find use in a variety of applications; they are especially useful for the detection and measurement of multiple targets in a single assay.

Beside applications that utilize ligands, the binding partners of the targets, as reporter molecules to mediate measurement for the targets, the invention also can be applied to utilize antagonists of the targets to mediate the measurement for the target in a competition assay format. In addition, the invention can be applied to utilize specific substrates to measure enzymatic activities of the targets.

The following preferred embodiments and examples are offered by way of illustration and not by way of limitation.

The subject invention provides a detection method for detection and quantitation, preferably, simultaneously detection and quantitation, of multiple immobilized targets.

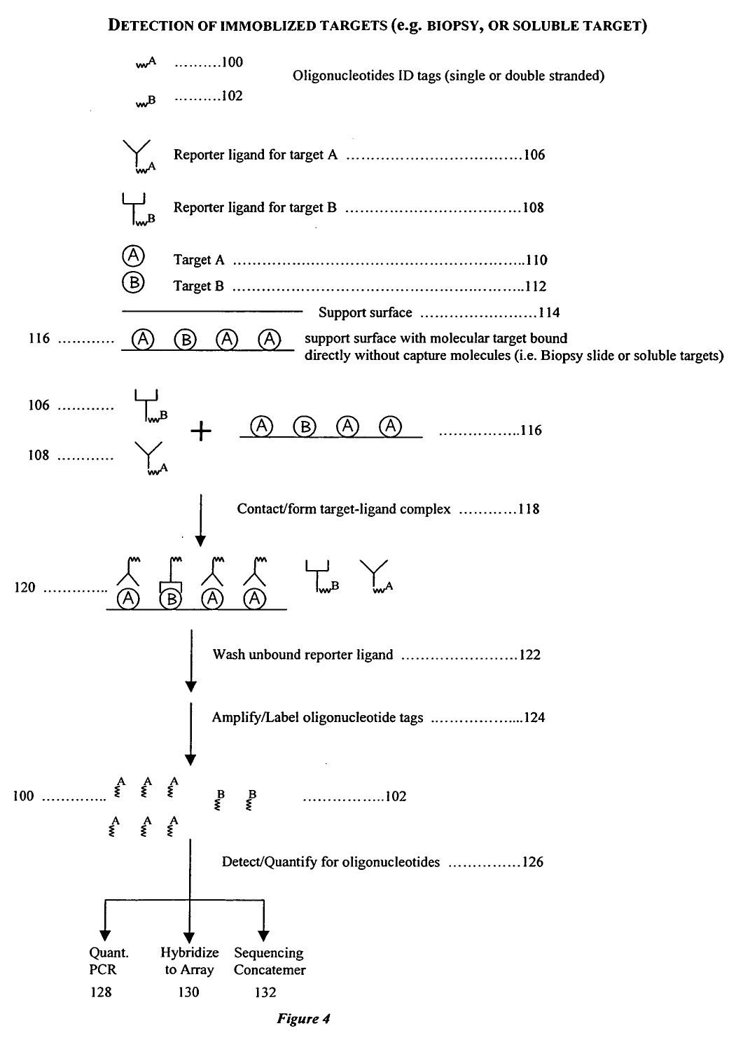

In a preferred embodiment, multiple targets are in fixed cells or tissue section. At step 1) incubating a plurality of oligonucleotide ID tag coded reporter ligands with fixed cells or tissue section, reporter ligands bind specifically to their targets in fixed cell or tissue section to form complexes in immobilized phase. Next at step 2) washing away unbound soluble reporter ligands from the immobilized cells or tissue section, the reporter ligands that bind with their targets are retained on the fixed cell or tissue section. Illustrated in FIG. 4 is an example of separation of the target bound reporter ligand in fixed cell or tissue section by washing away unbound soluble reporter ligands. At step 3) the oligonucleotide ID tags associated with immobilized cell or tissue section are amplified using any of a number of nucleic acid replication methods. And finally, at step 4) the amplified oligonucleofide ID tags are detected simultaneously using any of nucleic acid detection methods, such as hybridizing to nucleic acid array, parallel real-time quantitative PCR, nucleic acid sequencing, electrophoresis and chromatography.

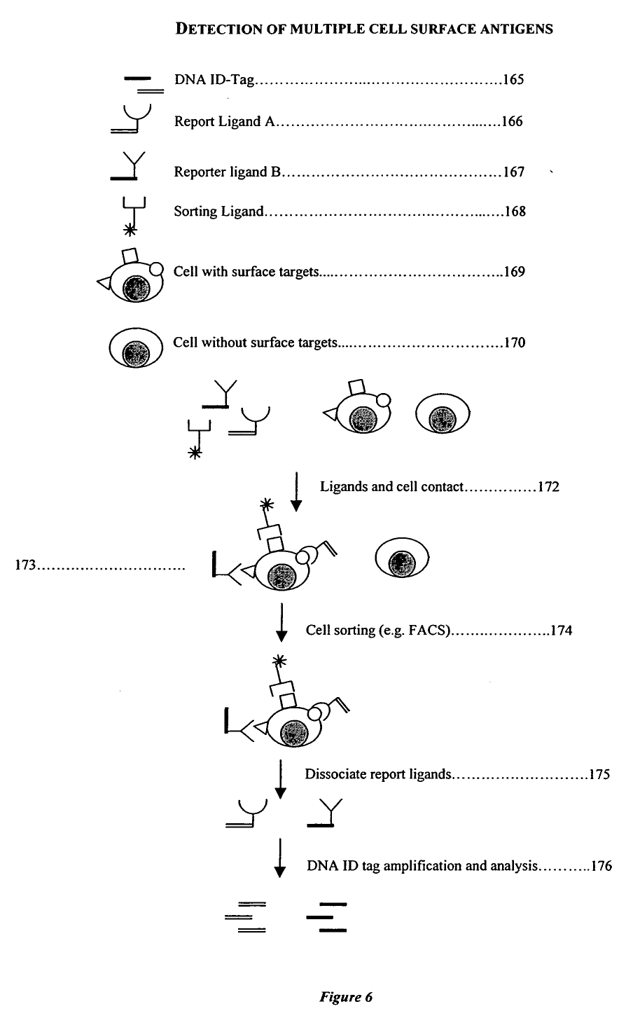

In another preferred embodiment, multiple targets are on the surface of a cell. Illustrated in FIG. 6 is an example of detection of multiple cell surface antigens. At step 1) incubating a plurality of oligonucleotide ID tag coded reporter ligands with cells, reporter ligands bind specifically to their targets to form complexes on a cell surface. Next at step 2) separating cells from unbound soluble reporter ligands using any of a number of methods, including but not limit to centrifugation, flow cytometry, precipitation and immobilization. Illustrated in FIG. 6 is an example of separation of the cell surface antigen bound reporter ligands from unbound soluble reporter ligands. At step 3) the oligonucleotide ID tags associated on cell surface are amplified using any of a number of nucleic acid replication methods. And finally, at step 4) the amplified oligonucleotide ID tags are detected simultaneously using any of nucleic acid detection methods, such as hybridizing to nucleic acid array, parallel real-time quantitative PCR, nucleic acid sequencing, electrophoresis and chromatography.

The subject invention also provides a detection method for detection and quantitation, preferably, simultaneously detection and quantitation, of multiple targets in a fluid. With soluble targets and soluble reporter ligand, the critical step of the embodiment is the separation of target bound reporter ligand from unbound soluble reporter ligand. A number of separation schemes may be employed.

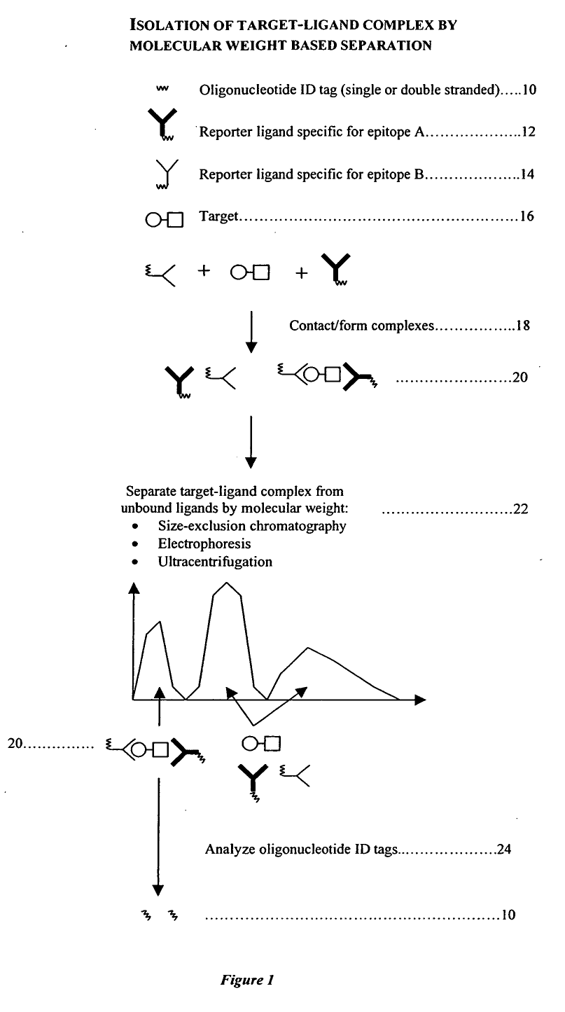

In a preferred embodiment, the method comprises at step 1) mixing a plurality of oligonucleotide ID tag coded reporter ligands with targets in solution phase, reporter ligands bind specifically to their targets to form complexes. Next at step 2) the complexes comprising reporter ligands and targets are separated from unbound reporter ligands utilizing a size differentiation methods, including but not limit to chromatography, electrophoresis, filtration and centrifugation. Illustrated in FIG. 1 is an example of separation of the reporter ligand-target complexes from unbound reporter ligand by size-exclusion chromatography. At step 3) the oligonucleotide ID tags associated with the complexes are either isolated and/or amplified using any of a number of nucleic acid replication methods. And finally, at step 4) the amplified oligonucleotide ID tags are detected simultaneously using any of nucleic acid detection methods, such as hybridizing to nucleic acid array, parallel real-time quantitative PCR, nucleic acid sequencing, electrophoresis and chromatography.

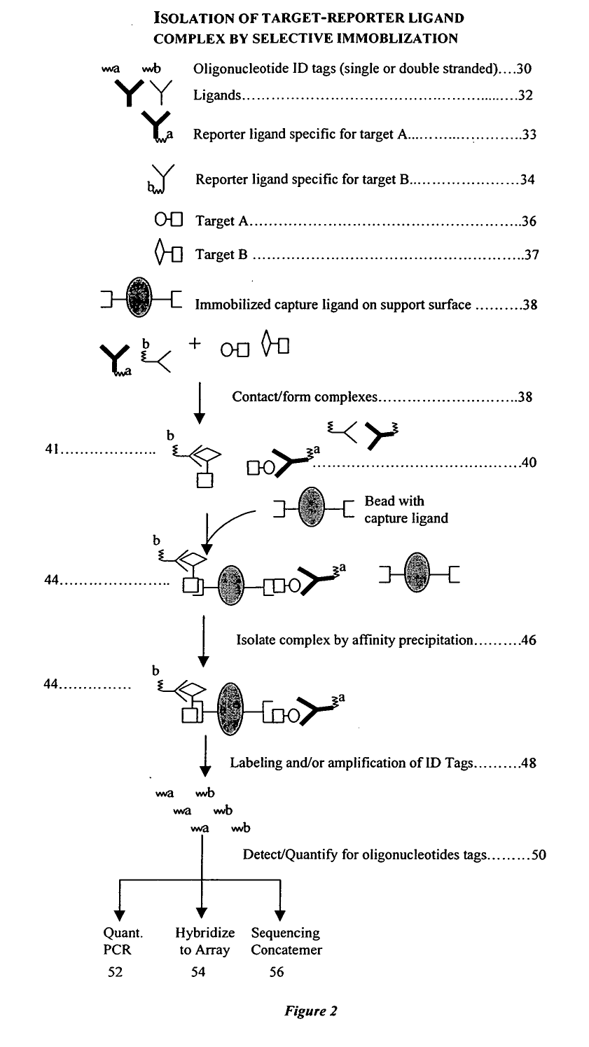

In another embodiment of subject invention, the multiple targets are present in solution phase, at step 1) mixing a plurality of oligonucleotide ID tag coded reporter ligands with targets, reporter ligands bind specifically to their targets to form complexes in solution phase. Next at step 2) the complexes comprising reporter ligands and targets are separated from unbound reporter ligands by selective immobilizing the complexes through specific binding between immobilized capture ligands and targets in complexes. The capture ligand may be single ligand that specifically binds with a common moiety or a structure feature shared by multiple targets, or may be a mixture of ligands that each specifically bind with a target. Illustrated in FIG. 2 is an example of separation of the reporter ligand-target complexes by selective immobilizing a complex to a support and washing away soluble unbound reporter ligand. At step 3) the oligonucleotide ID tags associated with the complexes are either isolated from support and/or amplified using any of a number of nucleic acid replication methods. And finally, at step 4) the isolated and/or amplified oligonucleotide ID tags are detected simultaneously using any of nucleic acid detection methods, such as hybridizing to nucleic acid array, multiplex real-time quantitative PCR, nucleic acid sequencing, electrophoresis and chromatography.

In another preferred embodiment, soluble targets are directly immobilized on a support without target-specific capture ligand. At step 1) the soluble targets are immobilized on a support surface, the chemical or physical property of support surface material captures targets from solution phase to immobilized phase. Next at step 2) a plurality of oligonucleotide ID tag coded reporter ligands contact with the immobilized targets to form reporter ligand-target complexes on the support surface. At step 3) unbound soluble reporter ligands are washed away from support surface. At step 4) the oligonucleotide ID tags associated with the complexes are either isolated from support and/or amplified using any of a number of nucleic acid replication methods. And finally, at step 5) the isolated and/or amplified oligonucleotide ID tags are detected simultaneously using any of nucleic acid detection methods, such as hybridizing to nucleic acid array, multiplex real-time quantitative PCR, nucleic acid sequencing, electrophoresis and chromatography.

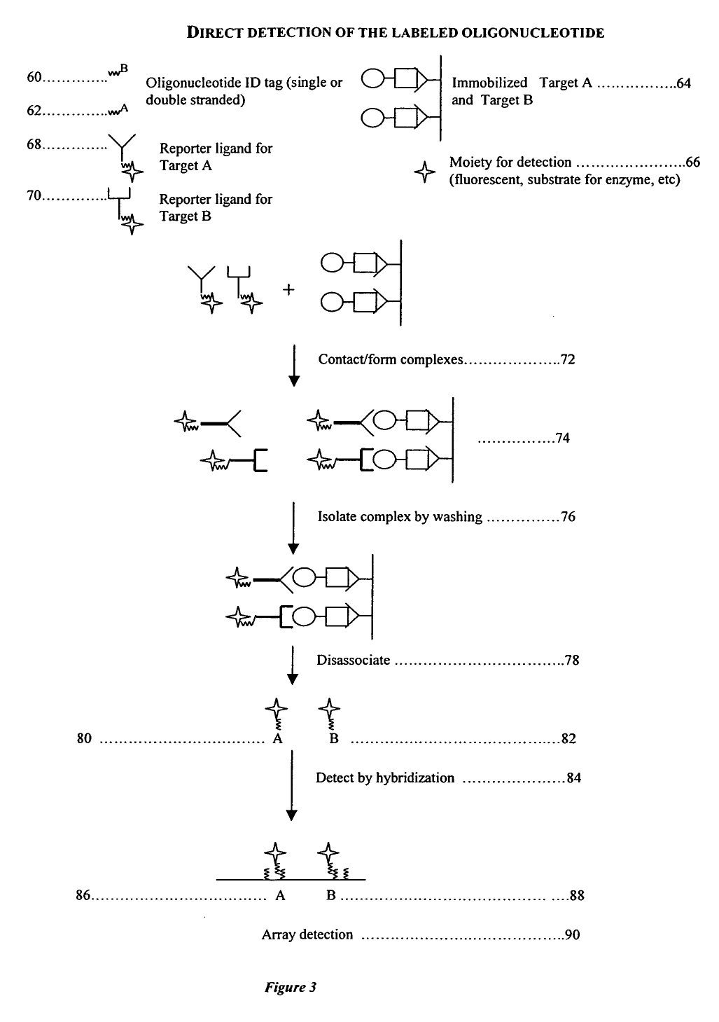

In another preferred embodiment, soluble targets are immobilized to a support through binding with an immobilized single capture ligand. As illustrated in FIG. 3, at step 1) the soluble targets are contacted with a support surface that immobilized with a single capture ligand, wherein the capture ligand is specific for a shared common moiety or a structure features present in the targets. The soluble targets are specifically immobilized on the support through binding with the capture ligand. Next at step 2) a plurality of oligonucleotide ID tag coded reporter ligands contacts with the immobilized targets to form reporter ligand-target complexes on the support. At step 3) wash away unbound soluble reporter ligands from the support surface. At step 4) the oligonucleotide ID tags associated with the complexes are either isolated from support and/or amplified using any of a number of nucleic acid replication methods. And finally, at step 5) the isolated and/or amplified oligonucleotide ID tags are detected simultaneously using any of nucleic acid detection methods, such as hybridizing to nucleic acid array, multiplex real-time quantitative PCR, nucleic acid sequencing, electrophoresis and chromatography.

In another preferred embodiment, soluble targets are immobilized to a support substrate through binding with the immobilized mixture of capture ligands. At step 1) the soluble targets are contacted with a support that immobilized with a mixture of capture ligands, wherein each capture ligand in the mixture is specific to a target. The soluble targets are specifically immobilized on the support through binding with capture ligands. Next at step 2) a plurality of oligonucleotide ID tag coded reporter ligands contact with the immobilized targets to form reporter ligand-target complexes on the support surface. At step 3) unbound soluble reporter ligands are washed away from support surface. At step 4) the oligonucleotide ID tags associated with the complexes are either isolated from support and/or amplified using any of a number of nucleic acid replication methods. And finally, at step 5) the isolated and/or amplified oligonucleotide ID tags are detected simultaneously using any of nucleic acid detection methods, such as hybridizing to nucleic acid array, parallel real-time quantitative PCR, nucleic acid sequencing, electrophoresis and chromatography.

The subject invention also provides competition method for analysis of multiple targets in solution. Insoluble targets may also be analyzed by competition method after the insoluble targets are solubilized.

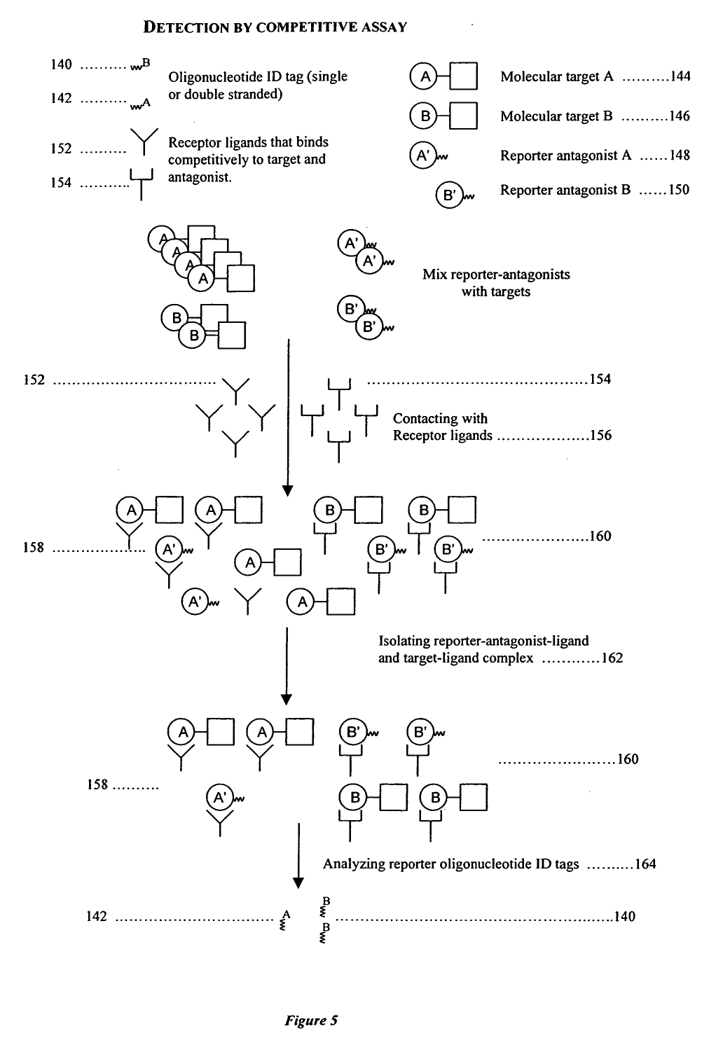

In a preferred embodiment, as illustrated in FIG. 5, competition method comprises at step 1) mix known amount of a plurality of oligonucleotide ID tag coded reporter antagonists with targets in solution phase, wherein each reporter antagonist capable of competing with a target for binding with a receptor ligand. At step 2) add limited amount of receptor ligands into solution to form complexes with both reporter antagonists and targets in solution, wherein each receptor ligand is specific for a target and its antagonist. At step 3) separate the unbound reporter antagonists from complexes of both reporter antagonist-receptor ligand and target-receptor ligand by immobilizing the complexes on a support surface, wherein a capture ligand specific to a shared common moiety or a structure feature present in all of receptor ligands is immobilized on the support surface. At step 4) the oligonucleotide ID tags associated with the complexes are either isolated from support surface and/or amplified using any of a number of nucleic acid replication methods. And finally, at step 5) the isolated and/or amplified oligonucleotide ID tags are detected simultaneously using any of nucleic acid detection methods, such as hybridizing to nucleic acid array, parallel real-time quantitative PCR, nucleic acid sequencing, electrophoresis and chromatography.

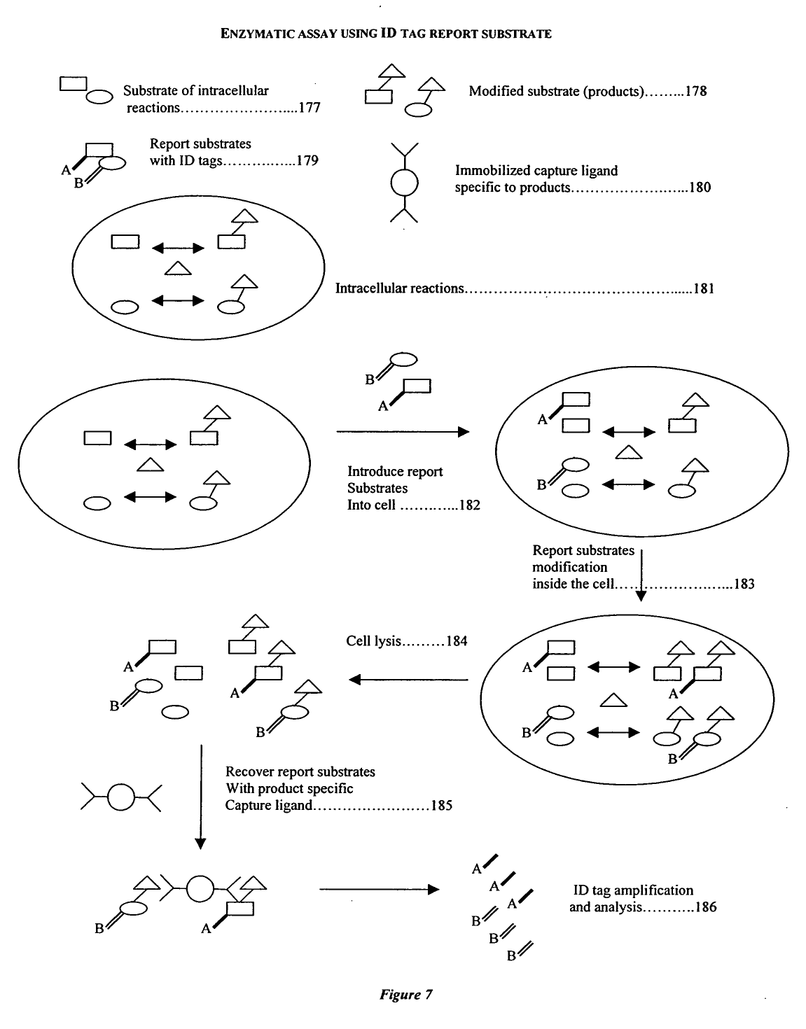

In addition, the subject invention also provides method for analysis of multiple enzyme activities in test tube or in living cells. In a preferred embodiment, as illustrated in FIG. 7, the method comprises at step 1) contacting a plurality of oligonucleotide ID tag coded reporter substrates with target enzymes in living cells via transfection or in a test tube, wherein the reporter substrates are modified enzymatically in the living cell or in a test tube. At step 2) the enzyme modified reporter substrates are isolated by addition of an immobilized capture ligand and washing away unmodified reporter substrate, wherein the capture ligand is specific for the modification moiety or the modified substrates. At step 3) the oligonucleotide ID tags associated with the modified reporter substrates are either isolated from support surface and/or amplified using any of a number of nucleic acid replication methods. And finally, at step 4) the isolated and/or amplified oligonucleotide ID tags are detected simultaneously using any of nucleic acid detection methods, such as hybridizing to nucleic acid array, parallel real-time quantitative PCR, nucleic acid sequencing, electrophoresis and chromatography.

Additionally, one of ordinary skill will recognize that the above several embodiments could be practiced employing alternative immobilization points through the assay.

The method can further comprise performing a control experiment by adding a known amount of one or more reference targets to the test sample together with unknown targets and detecting an amount of unique identifier nucleotide sequence representing the reference target in order to calibrate detection of the unknown targets.

The invention includes compositions and methods for simultaneous detecting multiple targets in a test sample. Whether the targets are insoluble, targets are soluble in a fluid, targets are immobilized on a solid support, or targets that compete with an antagonist reporter for a binding site, the targets can comprise any substance for which a binding ligand can be developed or synthesized or identified. The targets can be broadly construed as any moiety biological or otherwise comprising a molecular component and possibly comprising multiple molecules, aspects, elements, sides chains and the like; targets can be complexes of more than one molecule or target; targets can comprise a binding site on a molecule, aspect, element, side chain or the like; targets can be whole or part of an organism, cell, virus, bacterium; targets can comprise an analyte or substance which it is desirable to analyze. For example, also, a target may include, but is not limited to, a peptide, a polypeptide, a protein, an antibody, an antigen, a ribonucleotide, a deoxynucleotide, a polynucleotide, a lipid, a saccharide, a polysacchride, a hapten, a sugar, any other organic ligand, a polymer, a subcellular organelle, a cell, tissue, a microorganism, a virus, a moiety, a motif, a fragment, a complex, and a product of any of these, or broadly any substance for which a binding ligand can be developed or synthesized or identified.

The targets can be, for example, selected from the group consisting of naturally occurring molecules, synthetic molecules, a domain, a motif, epitope, a moiety of a molecule, a complex of molecules, proteins, polypeptides, peptides, post-translationally modified proteins, post-translationally modified polypeptides, glycoproteins, protein complexes comprising more than one protein, antigens, phosphorylated proteins, antibodies, antibody fragments, single chain antibodies, phage displayed antibodies, lectins, lipids, carbohydrates, small organic molecules, polymers, sugars, oxy sugars, deoxy sugars, phosphorylated oxy sugars, phosphorylated deoxy sugars, polymers, mixtures of polymers, saccharides, monsaccharides, polysaccharides, nucleic acids, ribonucleic acids, deoxyribonucleic acids, polynucleotides, methylated DNA, carbohydrates, small organic molecules, amino acids, steroids, modified steroids, fatty acids, whole cells, micro-organisms, bacterial organisms, viral organisms, bacterial proteins, viral proteins, secreted molecules, cell surface proteins, subcellular organelles, nuclear proteins, complexes thereof, naturally occurring forms thereof, synthetic forms thereof, derivatives thereof, combinations thereof, and metabolites of biological processes.

In practicing the subject invention, the methods require contacting reporter ligands with the targets. Each reporter ligand specifically binds a target. Specific binding constitutes an ability of the ligand to bind one target and not other targets. Specific binding also refers to the different degree of selectivity of a ligand binding with a target. The reporter ligands comprise a ligand attaching with an oligonucleotide ID tag.

In general, a ligand refers to a substance capable of specific binding with a desired target, such as the molecular and other targets identified herein. Thus a ligand can include, for example, but is not limited to a compound, a peptide, a polypeptide, a protein, an antibody, an antigen, a ribonucleotide, a deoxyribonucleotide, a polynucleotide, a lipid, a sacchride, a polysacchride, a hapten, a sugar, a toxin, a therapeutic agent, an organic ligand, a polymer, a sub cellular organelle, a cell, tissue, a microorganism, a virus, a viral moiety, a motif, a fragment, a complex, a product of any of these in a biological or other context, a natural form or a modified form of any of these, an artificial imprint of a polypeptide, a polyribonucleotide, a lipid, a carbohydrate etc. Antibodies can include whole antibodies, antibody fragments such as Fab, single chain antibody, for example. Antibodies can be from various species such as chicken, rabbit, mouse, human, bird, reptile, mammal, and in general any organisms capable of generating an antibody. Antibodies can also be constructed artificially using recombinant DNA technology and be produced in vivo and in vitro.

Accordingly, the ligands can be selected from the group consisting of naturally occurring molecules, e.g., antibodies, antibody fragments, single chain antibodies, phage displayed antibodies, polynucleotides; in vitro evaluated oligonucleotide, e.g., aptamers (Brody E N and Gold L. J. Biotechnol, Mar; 74 (1):5-13, 2000 and Jayasena S D Clin Chem. Sep; 45 (9): 1628-50), in vitro evaluated polypeptide, e.g., polypeptide evolved by ribosome display (Hanes and Pluckthun, Proc. Natl. Acad. SCI. 94, 4937, 1997 AND Ryabova, etc. Nature Biotechnology, 15, 79, 1997), artificial imprint of target or target analog, e.g., protein print described by Aspira Biosystems (San Francisco, Calif.), synthetic molecules, natural peptides, modified forms of peptides, modified forms of polypeptides, proteins, natural proteins, modified forms of proteins, post-translationally modified peptides, post-translationally modified polypeptides, post-translationally modified proteins, natural nucleotides, modified nucleotides, modified polynucleotides, post-transcriptionally modified nucleotides, post-transcriptionally modified polynucleotides, natural lipids, natural polylipids, modified lipids, modified polylipids, natural saccharides, natural polysaccharides, modified saccharides, modified polysaccharides, cells, cell lysates, a micro-organism, a virus, polymers, mixtures of polymers, polypeptides, glycoproteins, protein complexes comprising more than one protein, antigens, phosphorylated proteins, lectins, lipids, carbohydrates, small organic molecules, polymers, sugars, oxy sugars, deoxy sugars, phosphorylated oxy sugars, phosphorylated deoxy sugars, saccharides, monosaccharides, polysaccharides, whole cells, nucleic acids, ribonucleic acids, deoxyribonucleic acids, polynucleotides, methylated DNA, lipids, carbohydrates, polymers, mixtures of polymers, small organic molecules, amino acids, steroids, modified steroids, fatty acids, whole cells, micro-organisms, bacterial organisms, viral organisms, bacterial proteins, viral proteins, secreted molecules, cell surface proteins, subcellular organelles, nuclear proteins, complexes thereof, naturally occurring forms thereof, synthetic forms thereof, derivatives thereof, combinations thereof, and metabolites of biological processes. Broadly, a ligand for the assay, for making a reporter ligand, can be any ligand that specifically binds a particular target (e.g., such as but not limited to the targets listed herein) selected to be detected in an assay of the invention.

A reporter ligand can be formed by attaching an oligonucleotide ID tag to a ligand. The oligonucleotide ID tag can comprise a unique identifier nucleotide sequence. The unique identifier nucleotide sequence can comprise one or more nucleotide differences. The nucleotide differences can include differences in nucleotide composition, differences in sequence order, or a combination of two or more of these differences. One or more nucleotide differences are determined in comparison with identifier sequences of other reporter ligands in a given assay or test system.

The oligonucleotide ID tag can be single stranded or double stranded. The oligonucleotide can be either a deoxyribonucleotide or a ribonucleotide in natural form or as a modified derivative. All reporter ligands specific for a target can comprise a same unique identifier nucleotide sequence regardless of an epitope specificity of the reporter ligands. The differences in nucleotide sequence of the unique identifier nucleotide sequences can comprise differences, for example, selected from the group consisting of one or more difference in the order of nucleotides sequence, one or more nucleotide substitutions, one or more additions of a nucleotide, and one or more elimination of a nucleotide, or one or more differences in the composition of nucleotides. The unique identifier nucleotide sequences can have about the same DNA melting temperature (Tm). Nucleic acid array-based and PCR detection systems require about the same Tm for all the unique identifier nucleotide sequences. Concatemer-based detection requires about the same length of nucleotide sequence for nucleotide identifier sequences.

The oligonucleotide ID tag can further comprise a modified base (or nucleotide derivative) that contains a moiety for direct detection or a moiety for indirect detection of said tag. The moiety for detection can comprise a moiety, for example, selected from the group consisting of a radioactive isotope that generates a detectable signal, a fluorophore that generates a detectable signal, a chromophore that generates detectable signal, and an electron-based detection. Most chromophorebased detection operates in a manner by which a substrate turns into a chromophore by enzymes linked to tags directly or indirectly. Electron-based detection is available as described in U.S. Pat. No. 6,268,149, U.S. Pat. No. 6,268,150, U.S. Pat. No. 6,265,155, and U.S. Pat. No. 6,264,825. Electron transfer can occur as a result of hybridization between a modified strand and a complementary strand, and can then be used as a detection signal for the presence of a particular sequence. Electron-based detection uses a modified nucleic acid that can generate an electron upon hybridization of tag with its complementary molecule. Other detection mechanisms can include an enzyme that catalyzes a substrate which generates chemiluminescence or colormetric detectable signal, biotin that binds avidin that can be attached with a fluorophore for generating a detectable signal or attached with a enzyme that catalyzes a substrate that generates a chemiluminescence or colormetric signal, digoxigenin for binding with anti-digoxigenin antibody conjugated with a fluorophore or an enzyme that catalyze a substrate to generate chemiluminescent or colormetric signal, fluorescein for binding with anti-fluorescein antibody that is conjugated with an enzyme or a fluorophore. See “Nonradioactive Labeling and Detection of Biomolecules” edited by C. Kessler and published by Springer-Verlag in 1992. In general, a medium molecule, such as, for example, biotin, digoxigenin and fluorescein can be used for this detection, and thus any medium molecule that provides a label and detectable signal analogous to those described above can be used. An example of a simple version of indirect detection is to have the detection moiety directly linked to avidin, the complement molecule to biotin. In addition there are other versions such as having naïve avidin binding to biotin first to turn one biotin site into multiple avidin sites (avidin is multi-valent) followed by adding biotin conjugated with a detection moiety or enzyme.

Providing reporter ligands can further comprise that all reporter ligands specific for a target comprise a same unique identifier nucleotide sequence regardless of an epitope specificity of the reporter ligand.

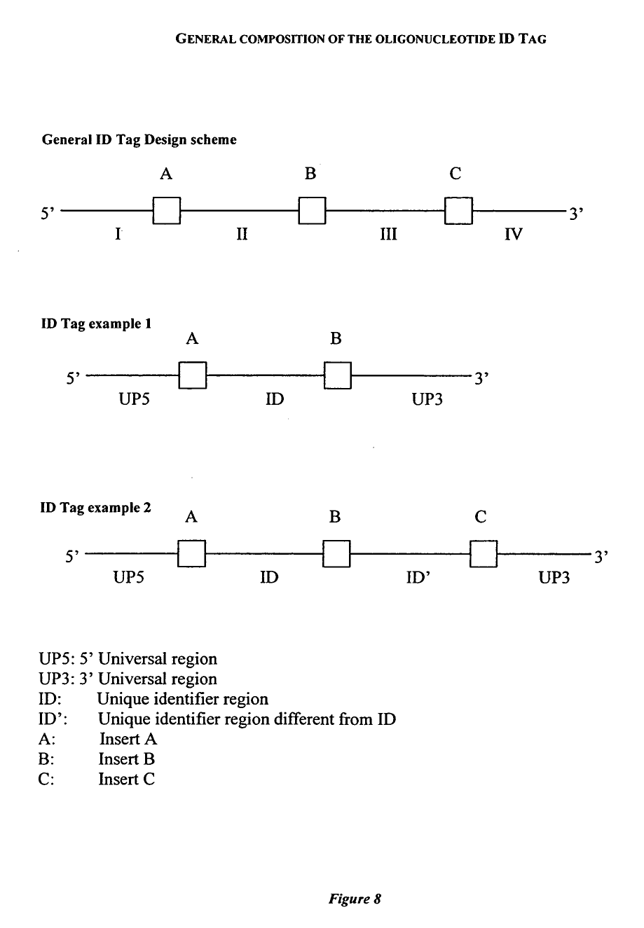

More specifically, and in more detail the invention is provided with the following details and elements. An oligonucleotide ID tag can comprise an oligonucleotide sequence that contains within its sequence one or more unique identifier nucleotide sequences or regions. The oligonucleotide ID tag can include or not include accessory regions. An oligonucleotide ID tag can comprise linear or circular oligomers of natural and modified monomers or linkages, including, for example, deoxyribonucleosides, ribonucleosides, anomeric forms, and the like, capable of specifically binding to a complement polynucleotide in a regular pattern of monomer-to-monomer interactions, such as, for example, Watson-Crick type of base pairing, base stacking, Hoogsteen or reverse Hoogsteen types of base pairing, or the like. The linkage between monomeric nucleotide units includes phosphoramidate bonds, thioester bonds or analogs, methylphosphonate etc. bonds or analogs thereof to form oligonucleotides. Additional linkage between monomeric nucleotide unites and modified oligonucleotide is described in publications such as by Milligan et al. (Concepts in antisense drug design in J. Med Chem 1993 Jul. 9; 36(14):1923-37); by Herdewijn P. (Heterocyclic modifications of oligonucleotides and antisense technology in Antisense Nucleic Acid Drug Dev 2000 August; 10(4):297-310); by De Mesmaeker A. et al. (Backbone modifications in oligonucleotides and peptide nucleic acid systems in Curr Opin Struct Biol. 1995 June; 5(3):343-55); by Gryaznov S M (Oligonucleotide N3′-->P5′ phosphoramidates as potential therapeutic agents in Biochim Biophys Acta. 1999 Dec. 10; 1489(1):131-40); by Micklefield J (Backbone modification of nucleic acids: synthesis, structure and therapeutic applications in Curr Med Chem 2001 August; 8(10):1157-79); by Sproat B S (Chemical nucleic acid synthesis, modification and labeling in Curr Opin Biotechnol 1993 February; 4(1):20-8) and elsewhere. Two types of oligonucleotide ID tag contain the general composition are illustrated in FIG. 8.

One type oligonucleotide ID tag contains only one unique identifier sequence; Another type of oligonucleotide ID tags contains two different unique sequences.

The unique identifier nucleotide sequence is the nucleic acid or nucleotide sequence that is unique in sequence identity, sequence composition or a combination of these. A unique identifier nucleotide sequence is used for sequence-based detection to detect the sequence that encodes the identity of the target. A measurement process is applied in order to identify a given target and to distinguish that target from other targets identified in the same assay. Such an assay identifies multiple targets in a parallel or simultaneous fashion.

Other sequence regions within the oligonucleotide ID tags can be called and considered accessory regions. Accessory regions can also refer to region comprising sequence that is not used for sequence-based decoding, or not used as a unique identifier sequence to identify an oligonucleotide ID tag, such as UP5, UP3 and Insert A, B, C as depicted above. For convenience, accessory regions can be designed to be the same among all oligonucleotide ID tags used in one assay. In general, accessory regions are sequences that can be either annealed with complementary sequences that facilitate replicating the oligonucleotide ID tag, incorporating modified nucleotide derivative into oligonucleotide ID tag, or annealing with fluorescence labeled probe for real-time quantitative PCR detection, or can be restrictive endonuclease cleavage sites. Accessory regions are optional depending on which method is used for decoding, amplification and measurement of the unique identifier nucleotide sequence portion of the oligonucleotide ID tag. Additional nucleotide sequences and accessory sequence regions may be included in an oligonucleotide ID tag depending on the methods employed to amplify and to detect the unique identifier nucleotide sequence.

The unique identifier nucleotide sequence may range in length from 10-1000 nucleotides (nt), or basepairs, usually from 15-500 nucleotides or basepairs, more usually from 20-250 nucleotides, or basepairs. Depending on the nature of synthesizing oligonucleotide ID tag, the chemical synthesized oligonucleotide ID tag may have a short unique identifier sequence in a range usually from about 12 to about 120 nucleotides or basepairs, more usually in a range from about 18 to about 40 nucleotides or basepairs. The oligonucleotide ID tag synthesized by a enzyme reaction or synthesized in a organism can have a longer unique identifier sequence in a range from usually 40 to 500 nucleotide or basepairs, more usually from 60 to 200 basepairs. The unique identifier nucleotide sequence can be designed accordingly to the decoding method selected and the amount of diversity of targets. For hybridization based sequence detection, such as nucleic acid array, e.g. flat array, suspension sphere array, or a bundle of fiber array, the unique identifier nucleotide sequences should be as different as possible and the melting temperature (Tm) of the unique identifier nucleotide sequences should be as similar as possible. The same melting temperature and maximum difference in sequences among all unique identifier sequences used in one assay allows design of a washing stringency to ensure maximum specific annealing between a unique identifier nucleotide sequence and its complementary nucleotide sequences in a hybridization reaction without significant cross-hybridization. This in turn ensures more specific and accurate measurement for the quantity of each unique identifier nucleotide sequence. Generally, the number of mismatches among unique identifier sequences in one assay should contribute to at least 5° C. difference in washing temperature comparing to perfect match, more usually at least 15° C. difference or larger. Note that the larger the sequence difference is among unique identifier sequences, the smaller the likelihood is of cross-hybridization occurring when hybridizing different unique identifier sequences to their complementary sequences. The specificity of base-pairing is higher, and thus detection can be more specific.

When parallel PCR is designed for detection of unique identifier nucleotide sequence, the unique identifier nucleotide sequence will anneal with a sequence-complementary PCR primer. The specific annealing between PCR primers and their complementary unique identifier nucleotide sequences determine the specificity of the detection. The unique identifier nucleotide sequences that complementary to PCR primers are usually at least 8 nt, more usually at least 16 and may be as long as 25 nt or longer, but will usually not exceed 50 nt. The number of mismatch among unique identifier nucleotide sequence is at least 1. When mismatch is at the 3′ end of its complementary PCR primer, one mismatch is sufficient to distinguish unique identifier nucleotide sequences, when the mismatch is not at the 3′ end of its PCR primer, more mismatches are need in order to determine an annealing temperature for specific parallel PCR detection.

For concatemer-based detection, unique identifier nucleotide sequence can be in the same length or similar in length to ensure the same ligation efficiency for concatemer formation, and the length of unique identifier nucleotide sequence usually not be longer than 50 nt, more usually not be longer that 20 nt. One nucleotide difference in unique identifier nucleotide sequences is sufficient to distinguish a unique identifier nucleotide sequence by concatemer-based sequencing detection.

In a preferred embodiment, a unique identifier nucleotide sequence can be designed by a subunit method as described in U.S. Pat. No. 5,635,400 or by a computer algorithm described by Shoemaker, et al. (Nature Genetics, 14:450-456 (1996)). To generate a collection of unique identifier nucleotide sequences to detect proteins from the whole genome of a given organism such as human, more than 35,000 different unique identifier nucleotide sequences are needed and these unique identifier nucleotide sequences can be designed according to the following publications: U.S. Pat. No. 5,635,400, U.S. Pat. No. 5,654,413, WO99/55886 and Shoemaker et al. (Nat Genet, 14(4):450-456 (1996)).

In another preferred embodiment, unique identifier nucleotide sequences can be the nucleotide sequences from naturally occurring DNA and RNA. To select naturally occurring nucleotide sequences to be unique identifier nucleotide sequence, the homologue of nucleotide sequences can be determine by BLAST (default setting). Generally, the homologues of nucleotide sequences are at least smaller than 70%, more usually smaller than 50%, preferably smaller than 20%.

The oligonucleotide ID tags may be synthesized by conventional oligonucleotide chemistry methods, where the nucleotide units may be: (a) solely nucleotides comprising the heterocyclic nitrogenous bases found in naturally occurring DNA and RNA, e.g. adenine, cytosine, guanine, thymine and uracil; (b) solely nucleotide analogs which are capable of base pairing in the course of nucleic acid replication or in hybridization condition annealing with complementary sequence such that they function as the above nucleotides found in naturally occurring DNA and RNA, where illustrative nucleotide analogs include inosine, xanthine, hypoxanthine, 1,2-diaminopurine and the like; or (c) from combinations of the nucleotides of (a) and nucleotide analogs of (b) the oligonucleotide ID tags may comprise detecting moiety or hapten groups, usually 1 to 2, which serve to simplify detection procedure.

The oligonucleotide ID tags may also be synthesized enzymatically by nucleic acid replication. The template for nucleic acid replication may be from naturally occurring DNA and RNA or derivatives from naturally occurring DNA and RNA. The template may also from chemically synthesized oligonucleotide with artificial nucleotide sequence or with a sequence homology to naturally occurring DNA and RNA. Nucleic acid replication may take place in test tube or may take place in organisms, e.g. E coli, yeast, virus. Any of a number of nucleic acid replication processes can be employed in enzymatically synthesis of oligonucleotide ID tags, e.g. PCR, LCR, in vitro transcription, T7 polymerase transcription, reverse transcription, or synthesized in vivo by bacteria or other organisms, such as synthesis in a plasmid, which are known by those skill in the art.

The oligonucleotide ID tags can also be synthesized together with the protein ligand or antagonist during in vitro protein synthesis by forming a RNA-protein fusion molecule as described in WO 01/16352 A1.

In accessory regions, UP5 and UP3, refers to adjacent universal sequences upstream and downstream of the unique identifier nucleotide sequences. They serve as the annealing templates for 5′ and 3′ universal primers. 5′ universal primer and 3′ universal primers can be used for priming nucleotide replication, such as polymerase chain reaction (PCR), T7 polymerase amplification, ligase chain reaction (LCR), rolling cycle amplification, strand displacement amplification, and cleavase/invader amplification, all of which can be employed for amplification of the unique identifier nucleotide sequences and for incorporating a moiety for detection into the oligonucleotide ID tag. UP3 and UP5 can also be used for priming real time quantitative PCR detection, or can be used for converting single stranded oligonucleotide ID tag into double stranded oligonucleotides. Insert A, B and C can be any sequences that serve as spacers between each region, a sequence for restriction cleavage, a sequence for annealing with primers or probes for amplification or detection. For example, if oligonucleotide ID tag has already been labeled with a moiety for detection, and amplification of oligonucleotide ID tags are not necessary, the oligonucleotide ID tag can be detected directly by hybridizing with a nucleic acid array, in this case, all accessory region, Up3, Up5, Insert A, B and C are not necessary, only ID or ID′ are needed. In another example, UP3 and UP5 accessory regions are necessary if a polymerase chain reaction is used to replicate oligonucleotide ID tags and to incorporate modified base into a unique identifier sequence, or to incorporate a primer that contains a modified base into oligonucleotide ID tags to facilitate the detection. In this example, it is preferable to include inserts A and B accessory regions to be two restriction sites, which facilitates cleavage of accessory regions from a unique identifier sequence after amplification and incorporating a moiety for detection into oligonucleotide ID tag. Removing accessory regions from a unique identifier sequence may increase specificity when detecting a unique identifier sequence by hybridizing to a nucleic acid array. In this case, it is also important to design the sequence of restriction site to exclude a modified base that is incorporated into oligonucleotide ID tag because a modified base may prevent the cleavage of oligonucleotide ID tags by restriction enzyme activity.

In other example, when modified base is included on a replicating primer, and the primer is used to introduce the moiety for detection into replicated copies of the oligonucleotide ID tag, the moiety for detection is located at an accessory region that is complementary to the primer. The accessory region should not be removed before hybridizing with nucleic acid array. For another example, when TaqMan™ real-time quantitative fluorescence PCR is used for decoding and quantification a target, the sequence at insert A position should be designed to complement with a common TaqMan™ probe sequence that contains a fluorescence dye and a quenching dye modified bases. In this example, UP3 and insert B are not necessary. In order to increase specificity for detection using real-time fluorescence quantitative PCR, another unique sequence region may be included in the accessory region, for example at the position of insert A. In this case, the quantitative PCR reaction are primed by two unique sequences flanking TaqMan™ probe annealing region, insert A should be designed for annealing with TaqMan™ probe and UP5, UP3 and insert B are not necessary.

In an additional example, if sequencing concatemers is going to be used for detection, UP3 or UP5 are necessary for converting single stranded oligonucleotide ID tags to double stranded ones, and inserts A and B should be designed as two restriction sites that can be cleaved by restrictive endonucleases to generate overhangs on both side of unique identifier sequences to facilitate ligation of unique identifier sequences together to form concatemers.

Oligonucleotide ID tags can also be designed to contain a modified base (or a nucleotide derivative) that contains a moiety for detection to facilitate direct detection of the oligonucleotide ID tag. The moiety for detection is a moiety that is capable of generating detectable signal directly from the moiety or indirectly through its binding to an intermediate molecule that is attached with a moiety that can generate a detectable signal. The binding between the modified base and the moiety that generates a detectable signal can be direct between modified base and moiety or indirect through one or more intermediate molecules. The detectable signal can include but is not limited to energy emitting, optical or electrical signals. The moieties that can be directly detected include, for example, radioactive isotopes such as, for example, ³²P, ³³P, ³⁵S, ¹²⁵I, ⁴C, ³H; fluorophores, such as, for example, Cy3, Cy5, fluorescein, Rhodamin, Texas Red and other derivatives; and chromophores, such as, for example, ruthenium derivatives which intercalate into DNA to produce photoluminescence under defined conditions (Friedman et al., Am. Chem. Soc. 112:4960 (1990). See also William T. Mason and W. T. Mason, in Fluorescent & Luminescent Probes: A practical Guide to Technology for Quantitative Real-Time Analysis, Academic Press, Inc. (1993)). A detection moiety that can be detected through coupling with an enzyme, an antibody, or a binding ligand that can be attached with a directly detectable moiety or enzyme, can include, for example, biotin, digoxigenin, and fluorescein. Fluorescein can serve for both direct and indirect detection. These moieties can bind an enzyme or fluorophore or chromophore linked to streptavidin and antibodies, for example. The enzymes that can be used to generate detectable signals include those that can catalyze a substrate to emit a chemiluminescent, a chemifluorescent or a chromogenic signal. Enzymes suitable for use in a detecting conjugates include, for example, but are not limited to, hydrolases, lyases, oxido-reductases, transferases, isomerases, ligases, peroxidase, glucose oxidase, phosphoatase, esterase and glycosidase. Specific examples include, for example, alkaline phosphatase, horse-radish peroxidase, lipases, beta-galactosidase, porcine liver esterase and the like. For indirect detection there can be other intermediate molecules between the labeled moiety such as biotin and molecules emitting a signal, for example, an ABC detection system (Vector Laboratory Inc, California) uses multivalent avidin first to bind to biotin, then uses biotin conjugated with a detection molecule such as an enzyme for detection. There can be two layers of biotin-avidin binding and this system can be used to amplify a signal several fold according to the valency of avidin molecule. Various non-radioactive detection methods are described elsewhere (see C. Kessler in Nonradioactive labeling and detection of biomolecules, Springer-Verlag, 1992).

In order to facilitate attachment of oligonucleotide ID tag to a ligand to form a reporter ligand, one or more modified bases can be incorporated into an oligonucleotide ID tag. The modified base can be directly introduced during chemical synthesis of oligonucleotide ID tag, or introduced by a primer that contains a modified base through priming enzymatic nucleotide synthesis. For example, thiol or amino-modified base on either 5′ or 3′ end can be used to facilitate coupling of an oligonucleotide ID tag to a protein based ligand by using different types of NHS-Esters-Maleimide crosslinkers, such as NBS, sulfo-SMCC, sulfo-MBS, SMPB, Sulfo-SMPB, GMBS, Sulfo-GMBS, EMCS, Sulfo-EMCS (products available from Pierce, Rockford, Ill.). Another example, a biotin modified base on either 5′ or 3′ end of oligonucleotide ID tag can be used to facilitate attachment of an oligonucleotide ID tag with a biotinylated ligand. The attachment can be bridged by an intermediate molecule such as avidin or streptavidin, or via a recombinant protein chimera, protein A-streptavidin for labeling biotinylated oligonucleotide to antibodies.

In general, one unique identifier nucleotide sequence can be assigned to one target. Multiple copies of the same or different unique identifier sequences can be included in one oligonucleotide ID tag. Multiple copies of oligonucleotide ID tags including the same unique identifier sequences can be included in one reporter ligand. If multiple ligands are used to detect one target, the oligonucleotide ID tag containing the same identifier sequence can be assigned to all ligands that bind to the same target. In this scenario, multiple ligands can be specific to the same target but different ligands can bind different epitopes on that target. One example is to assign one unique identifier nucleotide sequence to a polyclonal antibody that is a mixture of antibodies against different epitopes on a target. Another example is to assign one unique identifier nucleotide sequence to two or more monoclonal antibodies that are against different epitopes of a single target. When the goal is to accomplish parallel detecting of different epitopes, motif, binding sites and moieties on the same target, different unique identifier nucleotide sequences are assigned to different ligands that are specific to the different epitopes or binding sites or moieties on the same target.

Several methods can be used to attach an oligonucleotide ID tag to ligands to form reporter ligands. The 3′-end, 5′-end, or the middle portion of the oligonucleotide ID tag can be used for attaching the oligonucleotide ID tag to a ligand with or without a molecular spacer. The use of a spacer between the tag and the ligand is sometimes necessary to maintain the natural binding ability of the tag or ligand. An oligonucleotide ID tag can be covalently conjugated with a ligand directly, or though a mediator so as to be conjugated indirectly, for example, Hendrickson et al. (Nucleic Acid Research, 23(3): 522-529 (1995)) described the use of 5′ amino-modified oligonucleotides for antibody-oligonucleotide conjugation. Schweitzer et al. (PNAS 97:10113-10119, 2000) described a modified method to conjugate multiple oligonucleotides (3 on average) onto each antibody that serves as a reporter ligand. Oligonucleotide ID tags can also be attached with a ligand non-covalently or through a mediator. For example, Sano et al (Science, 258:120-122 (1992); BioTechnology, 9:1378 (1991)) constructed a protein A-streptavidin chimera protein capable of simultaneously binding antibody and biotinylated DNA label. Ruzicka et al. (Science, 260:698 (1993)) used commercially available avidin to join the biotinylated DNA label and biotinylated antibody. Zhou et al. (Nucleic Acid Research, 21:6038-6039 (1993)) employed streptavidin to link biotinylated DNA and antibody to form a universal reporter complex. In addition, oligonucleotide ID tags can be fused together with an in vitro translated protein as described in U.S. Patent WO 01/16352 A1. In the attachment process, the molar ratio of oligonucleotide ID tag to ligand should be one to one or more than one to one.

Once the reporter ligands have been formed, with their attached oligonucleotide ID tags comprising unique identifier nucleotide sequences, the reporter ligands for a given test sample will contact the test sample and the different targets present in that test sample. The chemical environment provided with this contact is sufficient to promote binding between the reporter ligands and the targets for which they are specific, and thus any additional reagents or conditions that need to be provided in order to promote specific binding between reporter ligands to the targets and to minimize non-specific binding of reporter ligands are provided during the contacting of the reporter ligands with the targets in the test sample. In a preferred example, the targets are proteins, conditions for an conventional immunoassay that promotes binding between an antibody and its antigen can be applied. Chemical environments suitable for binding antibody reporter ligand with protein targets will usually comprise buffering agents, usually in a concentration ranging from 10 to 200 mM which typically support a pH in the range 6 to 9, such as Tris-HCl, PBS, HEPES-KOH, etc; salts containing monovalent ions, such as KCl, NaCl, etc., at concentrations ranging from 0-1000 mM; salts containing divalent cations like CaCl₂, Mn(OAc)₂, MgCl₂ etc, at concentrations usually ranging from 0 to 20 mM; chalet, e.g. EDTA, EGTA and the like at concentrations usually ranging from 0 to 20 mM; and in some instance, include proteinase inhibitors, e.g. PMSF, leupeptin, trypsin inhibitors and the like; and additional reagents that blocking non-specific binding such as detergents, e.g. NP40, Tween 20, Trixton X-100 and the like; ionic detergents, proteins, e.g. albumin, animal serum, fat-free milk and the like; nucleic acid fragments, e.g. sperm DNA, yeast tRNA, synthetic oligonucleotide and the like. The chemical environment can be designed to be stringent enough to prevent non-specific binding. Detergents, pH change, ionic strength, temperature, and organic solvent, for example, can be used to change the stringency of the chemical environment. Tolerance to stringent conditions will vary with the nature of the target and reporter ligand, the contact conditions must be experimentally optimized for each assay for a given test sample. Enzymes that act as inhibitors for hydrolysis can also be included in the chemical environment to prevent the target or reporter ligand from hydrolysis during their contact. In one preferable example, when the targets are phosphorylation-modified residue, the chemical environment will also contain phosphatase inhibitors that prevent dephosphorylation of the phosphorylated target during contact with the reporter ligand. In another preferable example, when the targets are calmodulin binding proteins, calcium may be included in the chemical environment to promote binding between calmodulin with calmodulin binding proteins.

Depending on the nature of the targets in a test sample, the various chemical environment will be selected to promote the binding between reporter ligands and targets, and also to facilitate separation free unbound reporter ligands from reporter ligand-target complexes. In one preferable example, the targets are in fixed cells or fixed tissue section, detergents e.g. NP40, Triton X 100 will be included in the buffer medium. The detergents in buffer medium will permeablize cell membrane and facilitate reporter ligands contacting with the targets in the fixed cells. In another preferable example, the targets are on the cell surface, and the cell surface will be utilized as a support in separation of free unbound reporter ligands from reporter ligand-target complexes on the cell surface, the buffer medium should not contain detergents that may damage cell surface membrane.

The method and reagent for cell fixation and permeabilization are described in website and user manual of BDBioscience/PharMingen, Santa Cruz Biotech, Biosource and elsewhere. The method for tissue fixation is described in Cell Biology Laboratory Manual by Dr. William H. Heidcamp and elsewhere. The fixed and permeabilized cells and fixed tissue can then be subjected to interact with various ligands to detect the presence and the amount of various cellular molecules including cell surface and intracellular molecules.

Contacting reporter ligands with the targets in a test sample, the end result can be binding between reporter ligands and targets and the formation of complexes comprising a reporter ligand and a target. It is important to note that the complex can be a complex containing only one target and one reporter ligand, but can also be more than one of the same target binding to more than one of the same ligands or one target and multiple ligands. The complex can also be multiple different targets binding with multiple different reporter ligands, for example, multiple transcription factors are naturally together in a complex in the environment for contacting, reporter ligands specific for each transcription factor that binds with their counterpart in the complex to form a new complex that contains multiple transcription factors and multiple reporter ligands. In fact, many soluble proteins are naturally in complex form with other proteins in a fluid and thus need to remain in the complex for an accurate indication of their activity. In some cases more than one reporter ligand will bind a single target, depending on the binding sites that the reporter ligand binds at and whether the target possesses such a binding site specific for a given ligand. In order to obtain quantitative measurement for targets in a test sample, the amount of the reporter ligand should be in excess to its corresponding target, except in an antagonist competition assay (described below) in which, the antagonist reporter is not necessarily in an excess amount to its corresponding target.

Critical to the subject invention is separation of free unbound reporter ligands from reporter ligand-target complexes. Various separation schemes may be selected depending on the nature of the targets in a test sample. In general, free unbound reporter ligands are soluble, presenting in a solution phase. Targets can be present in soluble phase, e.g. IgGs, cytokines, hormone, cell lysate, etc. The targets can also present in insoluble phase e.g. cell surface proteins, transmembrane proteins in cell cultures or in the isolated cells, proteins in fixed cells or a tissue section. The targets in soluble phase can be immobilized onto a support surface to form immobilized targets, and the targets in insoluble phase can be, in some instance, solubilized into solution phase. Therefore, for any given sets of targets, depending on the nature of the targets, a contacting/isolation strategy can be designed to promote the reporter ligand-target interaction and to facilitate recovery of the reporter ligand-target complex.

In general, when the targets are in insoluble phase, the separation can be achieved by simply washing away unbound free reporter ligands from insoluble phase with a stringent solution that allows target-bound reporter ligands to remain in insoluble phase and dissociates non-specific bound reporter ligands from the insoluble phase.

A preferable embodiment, the targets are in a fixed cell or tissue section, the targets are in insoluble phase. The targets can be made available for binding with reporter ligands after a treatment that will permeablize the cell or tissue section. Incubating the cell or tissue section with a buffer medium containing reported ligands, the reporter ligands will bind specifically with their targets to form reporter ligand-target complex on the cell or tissue section, wherein the target bound reporter ligands retain on the insoluble phase. Washing the cell or tissue section with a stringent wash solution, the free unbound reporter ligands will be washed away from the cell or tissue section, and the target bound reporter ligands will remain on the cell or tissue section. The wash solution usually contains similar components as the buffer medium that describe above, providing a chemical environment that promote specific binding between reporter ligand to the target. In addition, the wash solution usually is more stringent than the buffer medium that used in the contacting reporter ligand to the target. Increased concentration of detergents, salts and the like are often used to increase wash stringency.

Another preferable embodiment, the targets are associated with cell membrane, the cell membrane can be used as insoluble phase to facilitate separation. In one embodiment, the targets are cell surface antigens. The cells, either fixed cells or living cells, will incubate with a buffer medium containing reporter ligands, resulting reporter ligands bind with their targets to form reporter ligand-target complex on the cell surface, wherein the target bound reporter ligand retain on the cell surface. Washing the cell surface with a wash solution, the free unbound reporter ligands will be washed away from the cells or from membrane fraction. In another embodiment, the targets are membrane proteins associated with membrane fraction of cells, the membrane fraction may be plasma membrane, nuclei membrane, mitochondria membrane, endoreticulum membrane or other cellular organelle membrane. The similar procedure used to detect cell surface antigens described above can be used to detect cell membrane associated proteins. The chemical components that may damage cell membrane shall be excluded from both the buffer medium for contacting and wash solution. When the cell is in suspension, several cycles of centrifugation and re-suspension of cells or membrane fraction is usually employed in wash procedure, when the cell is attached on the cell culture support surface, washing through the cells with wash solution can be easily apply. In a preferable application, reporter ligands will contact with fixed/permeabilized or live cells in a buffer medium that promote specific binding between reporter ligands and intracellular or cell surface antigens, before, during or after incubation with reporter ligands, a cell surface marker may be labeled with a fluorescence labeled antibody. The cells that bind with reporter ligands will be washed in a wash solution through several cycles centrifugation and re-suspension. The desired cell population with the fluorescent antibody labeling is then sorted out using a flow cytometer. The multiple cell surface antigens can be analyzed simultaneously in different cell types. In this application, cell sorting process in which the cell population of interest is captured with target specific fluorescent labeling and a cell sorter. It is noted that many other cell fractionation techniques can be used in place of cell sorting in this application. For example, the ligands against cellular targets can be immobilized on magnetic beads or other support surfaces, the desired cell population can be separated through magnetic beads separation, affinity ligand column by positive or negative selection. Sometimes the desired cell population can also be achieved by separation based on biophysical properties of the cell. Cells can be separated by density with gradient centrifugation, by forward/side scattering parameter with FACS sorting and by selective binding to support surface.

Examples of targets on cell membrane are CD antigen (example: CD1-247 found on BDBioscience user manual and website, and Leukocyte typing VII, by David Mason, et al., Published by Oxford University Press), adhesion molecule (example: E-selectin, L-selectin, P-selectin, integrin α1, integrin α2, integrin α3, integrin α4, integrin α5□integrin α6□integrin α7, integrin α8, integrin α9, integrin α10, integrin α1, integrin α_{IEL}, integrin α_{L}, integrin α_{M}, integrin α_{X}, integrin α_{V}, integrin α_{Iib}, integrin β₁, integrin β₂, integrin β₃, integrin β₄, integrin β₅, integrin β₆, integrin β₇, integrin β₈, BCM1, BL-CAM, ICAM-1, ICAM-2, ICAM-3, LFA-2, LFA-3, MCAM, NCAM, Neurothelin, PECAM-1, RNCAM, VCAM-1, CEA, DCC, Cadherin-5, E-Cadherin, M-Cadherin, N-Cadherin, P-Cadherin, R-Cadherin, Desmoglein, α-Catenin, β-Catenin, γ-Catenin, and others found on website of BDbioscience), receptor (EGF receptor, PDGF receptor and other membrane receptor as listed on website of Santa Cruz Biotechnology and elsewhere).

When targets are in solution phase, different strategies can be designed to isolate the complexes from unbound free reporter ligand in solution phase. One strategy can be designed to change the phase of the complexes into insoluble phase and keep unbound free reporter ligand in solution phase. For example, the complexes can be immobilized onto a support surface by selectively binding the complex to a support surface and leaving free unbound reporter ligand in solution phase. After complexes become immobilized, a stringent wash solution will remove non-specifically bound reporter ligands from the support surface. The washing stringency of wash solution can be designed to maintain the complexes formation by specific binding between the reporter ligands and the target while dissociate reporter ligands bound non-specifically from the complexes. Another strategy can be designed to separate the complexes from free unbound reporter ligand based on the difference of the mass between the complexes and free reporter ligand. In this instant, the difference in the mass can be distinguished using any of a number of conventional techniques, for example, using size exclusion chromatography or by electrophoresis or by filtrating through a size exclusion filter as known by those skill in the art.

Soluble targets are targets present in a freely flowing solution. The target can be originally soluble in solution phase or can be originally insoluble but later solubilized into solution phase. For example, targets present in cells, tissue, tissue section, animal, plant, and microbial contexts which are not soluble originally can be solubilized into solution phase by chemical or enzymatic treatment, such as, by extracting targets from a test sample with a solution containing detergents (for example SDS, Tween -20 or -80, TritonX-100 etc), chaotropic agents (for example Urea, SCN⁻, etc) or enzymes (for example, proteases, protease K, trypsin, papine, collagenase, endoglycosidase, exoglycosidase, peptidase, and the like). Soluble targets can also be targets present in body fluids, such as but not limited to, for example, urine, pleural fluid, pericardial fluid, peritoneal fluid, tears, cerebrospinal, synovial and serous body fluid, plasma, milk, sputum, fecal matter, lung aspirates, and exudates. Soluble targets can also be present in cell or tissue culture fluid, in microbial culture fluid, aerosols, crop materials, soils and ground water, for example, or in general in any fluid like medium.

An immobilized target refers to a target present in an immobile phase, for example, including an embedded tissue section comprising any one or more of fixed cells, tissues, microorganism, or organ samples, or immobilized cells or cell membrane fragments. In order to facilitate isolation of the reporter ligand-target complexes from free unbound reporter ligand after the contact, targets that are originally present in a soluble phase or solubilized into a solution phase can also be immobilized onto a support surface.

The support surface onto which immobilization (or other operations involving a solid surface described herein) occurs can be various in material. Such as, for example, glass, synthetic polymers (e.g. polystyrene, polypropylene, polyglycidylmethacrylate, aminated or carboxylated polystyrene, polyacrylamides, polyamides, polyvinylchloride), agarose, nitrocellulose, nylon, lipid, plasma membrane, metal and silicon and the like. The support surface (for immobilization and other operations of the invention) can also be various in formation and shape, such as solid, hallow, wafer or wafer like; an in the shape of flat, spherical, stick or rod-like, strips, microwells, microtubes, microfibers, or capillaries, etc. Many types of support surface may be purchased commercially from various sources, where such sources include Pierce, Nunc, Amersham, Sigma, VWR, Fisher etc.

Non-specific immobilization comprises affixing targets along with other surrounding entities onto a support surface. The affixing procedure for non-specific immobilization does not discriminate targets from surrounding ligands or other entities. For example, affixing a tissue section on a glass slide, a population of cells on a glass slide, coating a body fluid on beads or microwell plate, coating a soluble cell lysate, tissue lysate on beads or microwell plate, affixes both targets and other entities onto the support surface. Non-specific immobilization is accomplished through a physical interaction between the surface material and the targets and surrounding ligands or other entities that share similar physical properties with the target. The physical interaction that retains the targets and other entities on the support surface can be, for example, charge, hydrophobicity, hydrophilicity and the like, for example, interactions between proteins and polystyrene, or interactions between proteins and nitrocellulose. Non-specific immobilization can also be mediated by chemical reaction where the support surface contains an active chemical that forms a covalent bond with targets and perhaps other molecules as well, e.g. aldehyde-modified support surface can react with amino groups in proteins, or amino-based support surface can react with oxidization activated carbohydrate moieties in glycoproteins, or support surface containing hydroxyl groups can first react with bifinctional chemical reagents, such as N,N dissuccinimidyl carbonate (DSC), N-hydroxysuccinimidyl chloroformate, to activate the hydroxyl groups and react with amino-containing molecules such as proteins. By using appropriate types of support surface, one may immobilize the targets following the instruction provided by the manufactures of the support surface.

Specific immobilization is selectively affixing targets onto a desired support surface. Generally, the targets are selectively to be retained on the support surface than other surrounding molecules. Specific immobilization is through specific binding between targets with the capture ligands that have been immobilized on a support surface. The capture ligand can comprise the same molecule as described for a reporter-ligand, both capture ligand and reporter ligand bind to targets specifically, the difference is that the capture ligand is not attached to the oligonucleotide ID tag, and the capture ligand is used as a mediator for selectively immobilizing the target or a complex that contains target to a support surface to facilitate isolating a reporter ligand-target complex from free unbound report-ligand. Therefore, the capture ligand binds with target at different position or epitope from that of reporter ligand. Specific immobilization will tend to enrich targets on the support surface.

Several methods can be used to specifically immobilize targets. For example, a group of targets can be selectively immobilized onto a support surface through binding with an immobilized capture ligand that selectively binds with a shared common structure feature among targets, such as, for example, a common post-translational modification moiety, e.g. phosphotyrosine; a common epitope, common motif, e.g. calmodulin binding site; or a common binding site, e.g. protein A binding site; or the like. For example, a capture ligand can be an antibody against a posttranslational modification moiety that is present in a group of targets (e.g. an immobilized anti-phosphotyrosine antibody that can selectively immobilize a group of proteins that contain phosphotyrosin residue). The other posttranslational modification moieties include, for example, phosphotyrosine, phosphoserine, phosphothreonine, phosphohistidine, acetylated-lysine, etc. The specific moiety can also be, but is not limited to, a polysaccharide structure, a lipid modification, ubiquitination, and methylation, ADP-ribosylation, and other modification moieties as described in WO 0127624. For example, an antibody against phosphotyrosine, or phosphoserine, or phosphothreonine can be used to immobilize targets containing such a modification. In another example, lectin can be used as a single immobilizing agent for immobilizing targets with a specific polysaccharide moiety as described by David C. Kilpatrick (Handbook of Animal Lectins: Properties & Biomedical Applications, CRC Press, 2000 and E. J. Van Damme, Handbook of Plant Lectins Properties & Biomedical Applications, Wiley, John & Sons, Inc, 1998). In another example a capture ligand may be an antibody against an epitope shared by a group of targets e.g. immobilized calmodulin that can selectively immobilize calmodulin-binding protein through binding to calmodulin-binding motif For example, caldesmon, ryanodine, adducin, MARCK3, NAP22/CAP23, neuronal NO synthase, metabotropic glutamate receptor 7A, calpastatin, calpontin, neurogranin, twitchin kinase, calmodulin-dependent protein kinase C, titin kinase, and myosin light chain kinase can be immobilized by binding to an immobilized calmodulin. Another example is a helix-loop-helix motif that can dimerize with any helix-loop-helix-containing proteins, and thus can act as a capture ligand for helix-loop-helix containing proteins. In another example, the capture ligand is Protein A or Protein G, immobilized protein A/G can capture immunoglobulins through binding with their Fc region. Additional binding pairs that can be employed to selectively immobilize targets to a support surface include but is not limit to binding pairs such as streptavidin bound to a biotin labeled target (Ed Harlow and David Lane, Antibodies, A Laboratory Manual, Cold Spring Harbor Laboratory, 1988); anti-digoxigenin antibody bound to digoxigenin labeled targets, anti-fluorescein antibody bound to fluorescein labeled targets, anti-HA antibody bound to HA tagged proteins, and anti-GST antibody bound to GST fusion proteins. Immobilized nucleic acid can selectively immobilize nucleotide acid binding proteins, for example, a double stranded oligonucleotide selectively binds to transcriptional factors. Immobilized capture ligand can interact with a group of proteins such as transcription factors that can bind to a group of proteins to form a transcription complex. Immobilized ligand that binds with a group of receptors can act as a capture ligand. Immobilized receptors that bind to multiple ligands can act as capture ligands.

A group of targets can also be selectively immobilized on a solid surface by a group of immobilized capture ligands each being specific for one target as described in U.S. Pat. No. 5,985,548. The capture ligands in the group can be the same type of molecule but have different in epitope specificity to the targets. The capture ligands can also be combinations of different types of molecules, e.g. antibody and antigen, protein and nucleic acid, protein and lipid, etc Capture ligands can be made by combining an antibody, antigen and one or more polysaccharides to form a combined entity that acts as a capture ligand. The combination of capture molecules used is determined by what a group of targets that the experiment attempts to analyze. For example, a group of monoclonal antibodies each specific to a target in the test sample are immobilized on a support surface, such as antibodies for different cytokines. Each antibody can be capable of specifically binding with one cytokinee, and can selectively immobilize a different member of cytokines from a soluble sample to a support surface, so that multiple cytokines are captured on the support surface and can be analyzed simultaneously with subject invention. In another example, a mixture of combination of antibodies and antigens, each antibodies against a virus coating proteins and each viral antigens are capable of binding with antibodies generated by immunosystem to against virus infection, are immobilized on a support surface, can selectively immobilize viruses and virus-induced antibodies from a blood sample to a support surface, which can then be analyzed with subject invention simultaneously on the support surface. An immobilized group of oligonucleotides that are specific for a group of nucleic acid transcription factors can selectively immobilize a group of transcription factors from a cell lysate. Immobilized cell plasma membranes can selectively immobilize membrane-binding molecules.

In a preferable embodiment, both specific immobilization and non-specific immobilization are combined to obtained improved performance of subjected invention. At the first step the targets in the test sample will be enriched by selective binding to capture ligands and then immobilizing onto a support surface, at the second step the enriched immobilized targets will dissociate from the support surface, and finally enriched targets are non-specifically immobilized again on a support surface before contacting with reporter ligands. For example, the soluble capture ligand, e.g. a single or a group of polyclonal antibodies, each specific for a target or specific for a shared common epitope, are mixed with test sample in solution phase, the antibodies bind to targets in test sample to form antibody-antigen (targets) complexes, and antibody-target complexes can be precipitated by addition of protein A/G agarose conjugate into mixture. The targets that bind to antibodies are captured on protein A/G agarose beads through the affinity of protein A/G to Fc fragment of the antibody. The agarose beads that bound with targets can be washed to remove other entities in test sample, and the targets that captured on protein A/G agarose beads can then be dissociated from agarose beads. The dissociated targets are selectively enriched targets from test sample. The selectively enriched targets can be immobilized on another support surface again as described above to facilitate separation of reporter ligand-target complex from unbound free reporter ligands. This approach avoids immobilizing capture antibodies on the support surface, which often cause losing activity of antibodies. Capture antibodies can be mixed with targets in a solution phase, therefore the maximum activity of capture antibodies can be preserved. In addition, this approach allows capture antibodies bind with the same epitope that reporter ligand bind with. For example, polyclonal antibodies can be used as capture antibodies to capture targets to protein A/G agarose, by boiling agarose beads with a solution containing SDS and mercapto-ethanol or DTT, the targets are dissociated from capture antibodies as well as from agarose beads. The dissociated targets can then be immobilized onto nitrocellulose membrane. The reporter ligands that bind with the same epitopes that capture antibodies also bind with will contact with the immobilized targets for detection.

In general, when the targets are immobilized on a support surface, isolation of the target-reporter ligand complex can be accomplished by simply washing away unbound free reporter ligands from the support surface by a stringent wash solution that allows target-bound reporter ligands to remain in the support surface and dissociates non-specific bound reporter ligand from the support surface. When targets are in liquid phase, different strategies can be designed to isolate the complexes from unbound free reporter ligand in solution phase. One strategy is designed to change the phase of the complexes into a solid phase. For example, the complexes can be immobilized into a solid phase by selectively binding the complex to a support surface and leaving free unbound reporter ligand in the solution phase. After complexes are immobilized, washing the complexes in the solid phase with a stringent wash solution will remove non-specific bound report-ligands from the solid phase. The washing stringency can be designed to maintain the specific binding between target and reporter ligands and dissociate non-specific bound reporter ligands from the complexes. This will ensure that the detection for a unique identifier nucleotide sequence in the complex can be used as the measurement for the specific target.

Another strategy can be designed to separate the complexes from free unbound reporter ligand based on the difference of the mass between the complexes and free reporter ligand, in this instance, the difference in the mass can be conveniently distinguished by using a size exclusive chromatography or by electrophoresis or by filtrating through a size exclusion filter.

For example, separation of target-reporter ligand complexes from free reporter ligands can be accomplished when the reporter ligand binds to the target to form a complex based on their molecular weight difference. The complex is larger than free reporter ligand in molecular mass, e.g. (1 ligand+1 target) versus (1 ligand). In some instances, the target is present in a complex form, e.g. in plasma membrane or associated with other proteins or compounds (e.g. transcription complexes). The size comparison therefore is that one ligand and one target and the associated molecules) versus one ligand.

Multiple reporter ligands can be used to bind to one target on different binding sites, forming a complex that is significantly larger than the free reporter ligand itself. For example, a polyclonal antibody is a mixture of antibodies specific to different epitopes of an antigen. A target bound with a polyclonal antibody results in many IgG molecules binding to one target. The complex is significantly larger than a single IgG molecule, multiple IgGs and one target versus one IgG). Another example includes two monoclonal antibodies specific for two epitopes on a target protein. The mass of complex is equal to two IgGs and one target versus one IgG. The difference in molecular mass is a physical property that can assist in designing a method based upon mass difference in order to separate complexes from free reporter ligand. Various methods can be employed for molecular mass based separation, for example, but not limited to, size-exclusion chromatography (P. A. Miller, High Resolution Chromatography, Oxford University Press, 1999).

Size-exclusion chromatography can be used and exclusion limits of the resin can be chosen to be larger than free reporter ligand but smaller than the target/reporter ligand complex. For example, Macro-Prep SE 100/40 (Bio-Red, California), or Sephacryl S-200 or S-300, or Superdex 200 (Amersham Bioscience, NJ) can be chosen to separate free oligonucleotide labeled IgG (reporter ligand) from the target/IgG complex. Filtration can also be used to separate the target/reporter ligand complexes from free reporter ligand, in which the pore of the filtration membrane allows free reporter ligand to pass through but not bigger molecules or complexes, such as target/reporter ligand complexes, for example, using Centricon YM-100 spin filters (Millipore Corp, Bedford, Mass.). Preparative electrophoresis (Launch et al. Electrophoresis, 16:636-641, 1995; James P. Landers, Handbook of Capillary Electrophoresis, CRC Press, 1997) can also be employed for the separation. Preferably, electrophoresis is carried out in a native condition with slightly alkaline PH, it can be accomplished by gel electrophoresis or capillary electrophoresis (CE). Upon CE the complexes migrate slower than the free reporter ligands in the capillary, the fractions containing complexes larger than free reporter ligand can thus be collected. Centrifugation can also be employed for the separation. For example, by sucrose gradient ultra-centrifugation, the complexes that have larger molecular mass go to different layers of gradient. Preferably, the target is on the plasma membrane, or on a sub cellular organelle, and thus the complex that binds with reporter ligands is large enough to be simply precipitated by centrifugation.

Separation of reporter ligand-target complexes from free reporter ligands by affinity precipitation (immobilization) can be accomplished based upon the specific property of the target in the complex. The processes that selectively immobilize reporter ligand-target complexes to a support surface are the same processes as described for the specific immobilization of a target on a support surface as described above, and the capture ligand can also be the same as described above for specific immobilization of targets on a solid surface. A capture ligand that can selectively bind to a target can be first immobilized on a support surface through non-specific immobilization or specific immobilization (as described above). Then reporter ligand—target complexes can be selectively immobilized onto a solid surface through binding with a capture ligand that has been immobilized on the solid surface, and the free unbound reporter ligand and non-specifically bound reporter ligand can be washed off from the solid surface with a washing solution that is stringent enough to dissociate non-specifically bound reporter ligand from the support surface. For example, anti-phosphotyrosine antibody conjugated agarose beads can be used to affinity precipitate a subgroup of receptor tyrosine kinases-reporter ligand complexes. In another example, a capture ligand is not immobilized on the solid surface but conjugated with a binding intermediate moiety, such as biotin, digoxigenin, or fluorescein. So the capture ligand binds with target-reporter ligand complex in solution phase to form a complex that comprises a target, reporter ligand and capture ligand. The complex can then be immobilized to a support surface through the binding between a binding intermediate moiety and its binding pair molecule, such as, for example, avidin, streptavidin, anti-dixogenine antibody, or anti-fluoroscine antibody and the like, that has been immobilized on the support surface.

The support surface can be made of different materials or in different physical forms. (See description for solid surfaces in specific and non-specific immobilization above). In one embodiment, the capture ligand can be immobilized on agarose beads, and the complexes can be precipitated from solution by centrifugation; in another embodiment, the capture ligand is immobilized on a microtube or microtiter plate, and the complexes are retained in the microwell. Free reporter ligands are washed off. The method to immobilize capture ligands on a support surface can be the same as for the specific or non-specific immobilization. In one embodiment, biotinylated capture ligands can be immobilized on streptavidin-coated beads; in another embodiment, capture antibodies are coated on a microtiter plate. One capture ligand can be employed to immobilize all complexes when the capture ligand is capable of selectively binding with a shared common structural feature among targets, such as a common post-translational modification moiety, a common epitope, a common motif or a common binding site. The shared structural feature can be used to affinity precipitate the complex, as described above for specific immobilization of targets. In general, capture ligand should be supplied in excess of target in order to obtain a quantitative measurement for a target.

Detecting can comprise identifying a unique identifier nucleotide sequence and measuring a quantity of the unique identifier nucleotide sequence. Detecting can comprise direct detecting of the unique identifier nucleotide sequence that can be either dissociated from complex or direct detection can be accomplished with the unique identifier nucleotide sequence remaining associated with the complexes. For example, see the invader assay described in Hessner M J et al., Clinical Chemistry 46:1051-1056. (2000). Detecting can also comprise detecting replicated copies of the unique identifier nucleotide sequences. The oligonucleotide ID tags that are associated with or dissociated from the complexes can be replicated for detection.

In order to detect the oligonucleotide ID tags that attach to reporter ligands that bind to targets, oligonucleotide ID tags can be released from the complexes. In general, at least two approaches can be designed to release oligonucleotide ID tags into solution phase. One approach use the property that oligonucleotide ID tags that associated with the complexes can serve as templates for nucleic acid replication without need of dissociating from the complexes. The replicating enzymes, such as Taq polymerase, and primers, such as 5′ and 3′ universal PCR primers can be added directly into the mixture containing reporter ligand-target complexes, and nucleotide replication reaction is carried out using complexes associated oligonucleotide ID tags as templates. The replicated copies of oligonucleotide ID tags are released from complexes and present in solution phase, and are ready for sequence-based detection. Another approach use physical, chemical or enzymatic treatment that can either destroy the complexes by hydrolysis of non-nucleic acid components in the complexes, or dissociate oligonucleotide ID tags from the reporter ligands. In the case where double stranded oligonucleotide ID tags are conjugated with reporter ligand, increasing temperature can release the one strand of oligonucleotide ID tag that is not covalently bound with reporter-ligand. In addition, double stranded oligonucleotide ID tags can be released from complexes by endonuclease cleavage if a restrictive site is designed in the oligonucleotide ID tag for that purpose. For example, an oligonucleotide ID tag can be released from the complexes by an endonuclease, e.g. EcoR I, Bam H1, etc if restriction site is designed in the oligonucleotide ID tags in accessory region. An oligonucleotide ID tag can also be released by enzymatic cleavage for ligand, for example, treatment with trypsin, pepsin, or proteinase K that hydrolyzes proteins in the complex to release oligonucleotide ID tags. Chemical reaction, such as hydrolysis in HCl solution, or dissociate disulfide-bond between oligonucleotide ID tag and reporter ligand by reduction reagent, e.g. beta mercapto-ethanol, DTT can be used. Dissociation reagents sometimes affect the consequent reaction, such as nucleic acid amplification, labeling with detecting moiety and detection, etc. In order to neutralize or inactivate the dissociation reagents, heating can be used to inactivate enzymes by protein denaturation, or an enzyme inhibitor can also be used to inactivate enzymatic activities. For chemical reagent, adding a neutralization reagent can inactivate the reagent, e.g. alkaline can be used to neutralize HCl if it is necessary.

Also, site-specific cleavage can be created by using chemically modified DNA. There are a number of examples of compounds covalently linked to DNA that subsequently cause DNA chain cleavage. 10-phenanthroline has been coupled to single-stranded oligothymidylate via a linker that results in the cleavage of poly-dA oligonucleotides in the presence of Cu²⁺ and 3-mercaptopropionic acid (Francois et al., Biochemistry 27:2272 (1988)). Similar methods have been developed, e.g. use EDTA-Fe(II) for double stranded DNA (Boutorin et al., FEBS Lett. 172:43-46 (1986)), and triplex DNA (Strobel et al., Science 249:73 (1990)), porphyrin-Fe(III) (Le Doan et al., Biochemistry 25:6736-6739 (1986)), and 1,10-phenanthronine-Cu(I) (Chen et al., Proc. Natl. Acad. Sci. USA, 83:7147 (1986)), which all result in DNA chain cleavage in the presence of reducing agent in aerated solutions. A similar example using porphyrins mediated DNA strand cleavage, and base oxidation or cross-linking of the DNA under very specific conditions (Le Doan et al., Nucleic Acid Res. 15:7749 (1987)).

In general, before detection of unique identifier nucleotide sequence with any of a number of simultaneous sequence detection technologies, the oligonucleotide ID tags will be amplified and labeled with detecting moiety. Some exceptions are that if a detecting moiety is originally included in the oligonucleotide ID tag (see description above) the dissociated oligonucleotide ID tag can be detected directly by a sequence based-hybridization method, such as nucleic acid array based detection without further labeling procedure. If a detecting moiety is not included in the oligonucleotide ID tag, the dissociated oligonucleotide ID tag can still be detected by real time quantitative PCR.

Amplification and labeling can be accomplished in one step or in separate steps, e.g., labeling can be done after amplification. Labeling can be achieved by integrating modified nucleotide derivatives or by using a primer containing modified nucleotide derivative through polymerase reaction. Replicating the unique identifier nucleotide sequences can comprise performing a procedure selected from the group consisting of polymerase chain reaction, T7 polymerase amplification, strands-replacement amplification, ligase chain reaction, rolling cycle amplification, and other available methods of replication or amplification of nucleotide sequences. See, Clinical Diagnosis and Management by Laboratory Methods, by Henry et al., 20^{th} edition, published by John Bernard Henry, W.B. Saunders Company; Wu, et al., 1989, The ligation amplification reaction (LAR), amplification of specific DNA sequences using sequential rounds of template-dependent ligation, Genomics 4:560-569; Walker, et al., 1992, Strand displacement amplification, an isothermal, in vitro DNA amplification technique, Nucl Acids Res 1992, 20:1691-1696; Loeffler et al. 2001, Nucleic acid sequence-based amplification of Aspergillus RNA in blood samples, Journal of Clinical Microbiology, 39:1626-1629; 1056; Winn-Deen et al., 1993, Non-radioactive detection of Mycobacterium tuberculosis LCR products in a microliter plate format, Molecular and Cellular Probes 7:179-186), for example, in which the oligonucleotide ID tags are used as templates in order to quantify an amount of the unique identifier nucleotide sequences.

The replicated copies can be further amplified and labeled with a moiety for detection if necessary, e.g., Shoemaker et al., Nat Genet, 14(4):450-456, 1996; Winzeler et al., Science 285:901-906, 1999, Ausubel, et al., Current Protocols in Molecular Biology, John Wiley & Sons, Inc, 2001. In this embodiment, one of either the universal primer or dNTPs, preferably dNTPs, will be labeled such that the replicated copies of oligonucleotide ID tags are labeled. By labeled is meant that the entities comprise a member of a signal producing system and are thus detectable, either directly or through combined action with one or more additional members of a signal producing system. Examples of directly detectable labels include isotopic and fluorescent moieties incorporated into, usually covalently bonded to, a nucleotide monomeric unit, e.g. dNTP, or monomeric unit of the primer. Isotopic moieties or labels of interest include 32P, 33P, 35S, 125I, and the like. Fluorescent moieties or labels of interest include coumarin and its derivatives, e.g. 7-amino-4-methylcocumarin, aminocoumarin, bodipy dyes, such as Bodipy F L, cascade blue, fluorescein and its derivatives, e.g. fluorescein isothiocyanate, Oregon green, rhodamine dyes, e.g. texas red, tetramethylrhodamine, eosins and erythrosins, cyanine dyes, e.g. Cy3 and Cy5, macrocyclic chelates of lanthanide ions, e.g. quantum day, fluorescent energy transfer dyes, such as thiazole orange-ethidium heterodimer, TOTAB, etc. The detect moieties or labels may also be members of a signal producing system that act in concert with one or more additional members of the same system to provide a detectable signal. Illustrative of such moieties or labels are members of a specific binding pair, e.g. biotin, fluorescein, digoxigenin, antigen, polyvalent cations, chelator groups, and the like, where the members specifically bind to additional members of the signal producing system, where the additional members provide a detectable signal either directly or indirectly, e.g. antibody conjugated to a fluorescent moiety or an enzymatic moiety capable of converting a substrate to a chromogenic or chemiluminescence product, e.g. alkaline phosphatase conjugate streptavidin, HRP conjugated avidin.

Amplification of oligonucleotide ID tags and detecting amplified copies of oligonucleotide ID tags can significantly increase the sensitivity for target detection. The amplified copies of oligonucleotide ID tags can be analyzed using different sequence-based oligonucleotide identification and quantification methods. For example, polymerase-based quantification, nucleic acid array-based detection, or forming and sequencing concatemers can be used. T7 polymerase-based amplification (see Loeffler et. al. Journal of Clinical Microbiology, 39:1626-1629, 2001) generates single strand RNA copies of oligonucleotide ID tags. These RNA copies can be directly detected by hybridizing with a nucleic acid array, or can be reverse transcribed to cDNA and detected by other sequence-based detection methods for DNA, such as quantitative PCR, cPCR, and sequencing concatemers, and other methods.

The labeling process can serve both the function of labeling as well as amplification. Methods of nucleotide amplification can include, for example, PCR (as described in U.S. Pat. No. 4,683,195, and in U.S. Pat. No. 4,683,202), ligase chain reaction or LCR (as described in Tabor, S. and Richardson, C. C., 1985, PNAS 82:1074-1078), and rolling circle amplification (Schweitzer, et. al., 2000, PNAS 97:10113-10119), Some exemplary nucleic acid amplification techniques that can be used include T7 polymerase amplification, strand-displacement amplification, and other nucleic acid amplification technologies as described by John B. Henry, in Clinical Diagnosis and Management by Laboratory Methods, 20^{th} edition, W.B. Saunders Company, 2001. Any other methods that can replicate unique identifier nucleotide sequences at the same efficiency can be employed to practice the invention.

Detecting can comprise hybridizing the unique identifier nucleotide sequences with a array of complementary polymeric probes affixed on a support surface. A variety of different arrays that can be used are known in the art. The polymeric probes of the arrays may be oligonucleotides or hybridizing analogues or mimetics thereof, including nucleic acids in which the phosphodiester linkage has been replaced with a substitute linkage, such as phosphorothioate, methylimino, methylphosphonate, phosphoramidate, guanidine and the like; nucleic acids in which the ribose subunit has been substituted, e.g. hexosephosphodiester; peptide nucleic acids; an the like. The length of the nucleic acid probes will generally equal or smaller than oligonucleotide ID tags, usually from 10 to 1000 nts, more usually 15 to 200 nts in length, where the polynucleotide probes may be single or double stranded, usually single stranded, and may be chemically synthesized or PCR fragments amplified from DNA. The support surface can be selected from the group consisting of, for example, a planar solid support, a spherical solid support, a rod-like solid support, a tube-like solid support, a microwell, a microtube, and a capillary.

For nucleic acid array based detection, only the unique identifier nucleotide sequences need to be detected. If double strand copies of the unique identifier nucleotide sequence are used for hybridization, a step of denaturing double stranded DNA to single stranded DNA is necessary before hybridizing to a nucleic acid array. In one preferable embodiment, oligonucleotide ID tags can be designed to include a RNA promoter, e.g. T7 or S6 promoter, thus the unique identifier nucleotide sequence is replicated to be single stranded copies of RNA. In another preferable embodiment, the unique identifier nucleotide sequence is replicate by an asymmetrical PCR or uni-direction polymerase amplification; the unique identifier nucleotide sequence is replicated to be single stranded copies of DNA. In more preferable embodiment, either of one universal primer for PCR amplification, such UP5, or UP3 as described above, is synthesized to contain a phosphorothioate-modified nucleotide. The PCR replicates the unique identifier nucleotide sequence to be double stranded DNA. The double stranded DNA will under go an exonuclease treatment, one of the DNA strand that containing the phosphorothioate-modified primer is resistant to exonuclease, the another strand is degraded, thus only one single stranded replicated copies are remained for detection. The single stranded copies are ready to be detected by hybridizing to a nucleic acid array without denaturing.

Nucleic acid array based detection is a hybridization based nucleotide sequence identification and quantification method. Single stranded oligonucleotide ID tags or their replicated copies specifically bind to their complementary oligonucleotides that are affixed on a support surface. Because complementary nucleic acid are affixed at predetermined physical locations, the hybridization results in localizing the oligonucleotide ID tags or their replicated copies to the physical location on the support surface where their complementary sequence is affixed. If the oligonucleotide ID tags are labeled with a detection moiety, the measurement of the detection moiety that has localized on an array can reveal the relative amount of the oligonucleotide ID tag. Several types of nucleic acid array based detection methods can be employed for detecting multiple oligonucleotide sequences at the same time, for example, such as flat array, suspension beads array, optical fiber bundler array, e-Sensor, and others (Schena, M., DNA Microarrays, Oxford University press, 2001).

An nucleic acid array that is made by various methods and materials can be used to analyze unique identifier sequence, as described in Ramsay G, Nature Biotechnology 16:40-44, 1998, Okamoto, et al., Nature Biotechnology 18:438-441, 2000), or as provided by commercial vendors, such as Affymetrix (Santa Clara, Calif.), Motorola (Phoenix, Ark.), Mergen (San Leandro), Rosetta pharmaceuticals (Seattle, Wash.), Qiagen (Alameda, Calif.), Coming (Coming, N.Y.), NEN PerkinElmer Life Sciences(Boston, Mass.), Hyseq (Sunnyvale, Calif.), Luminex (Austin, Tex.), Illumina (San Diego, Calif.), Metrigenex (Gaitherburg, Md.), PamGene BV('s-Hertogenbosch, Netherlands) and Agilent (Palo Alto, Calif.) The hybridization condition for nucleic acid array-based detection is described elsewhere (see Shoemaker et al., Nat Genet, 1996 14(4):367-370), or described by commercial vendors. Either way, an optimal hybridization condition needs to be experimentally determined for particular test samples, targets to be tested or particular combinations of methods and techniques.

For isotope labeled oligonucleotides, radioactivity can be determined by autoradiography or by a phosphorimager. The signal intensity reflects the amount of oligonucleotide ID tag. For fluorophore-labeled oligonucleotide ID tags, fluorescence intensity can be detected by a fluorescence detector, such as an array scanner available from Affymetrix (Sunnyvale, Calif.) and from Axon Instruments Inc. (Union City, Calif.). When hybridizing with a suspension sphere array, fluorescent signal can be detected by flowcytometer or by Lumix2000 (Luminex Inc. Texas). For an oligonucleotide ID tag that is labeled with a moiety for detection, such as biotin or digoxin (DIG), or a moiety that can couple with an enzymatic reaction such as horse radish peroxidase (HRP) and alkaline phosphatase (AP), for example, the enzyme activity can be determined by measuring a chemiluminescent, fluorescence or colormetric signal that generates from a substrate reaction catalyzed by the enzymes. These enzyme based detection methods are described elsewhere (see C. Kessler Nonradioactive labeling and detection of biomolecules, Springer-Verlag, 1992). Oligonucleotide ID tags can also be detected by eSensor array (available from Motorola, Phoenix, Ariz.).

If the purpose is only to compare relative amounts of target between two samples, a single array can hybridize with two different fluorescent dye labeled oligonucleotide ID tags from two test samples. For example, Cy3 labeled oligonucleotide ID tags from sample 1 and Cy5 labeled oligonucleotide ID tags from sample 2. Equal amount of sample 1 and sample 2 are used to generate labeled oligonucleotide ID tags respectively. The relative amount of each target between two samples can be instantly determined based on the signal ratio of two fluorescent colors. This method has been used extensively in gene array technology for comparing gene expression between two samples and is described elsewhere (see Brown, et al., Nature Genetics Supplement 21:33-37, 1999) (see also example 2 to 6 below).

The same procedures used in flat nucleic acid array-based detection can be applied in a suspension array. Suspension type nucleic acid arrays that encode and decode by different methods can be used to analyze the unique identifier sequences. The physical location that determines the identity of an oligonucleotide on a two dimensional (flat) chip array is the X and Y axis, but with a suspension beads array, the physical location is coded by a detectable optical, isotopic, or other physical properties.

There are many encoding and decoding systems that can apply to this invention. For example, color encoding (see WO0114589), chemical encoding such as using peptide or small molecules (see Xiao, X. Y. and M. P. Nova in Combinatorial Chemistry; Synthesis and Application, Wiley, New York, 1996, chap. 7), oligonucleotide encoding (Walt, D. R. (2000). Techview:Molecular Biology. Bead-Based Fiber-Optic Arrays, Science, 287, 451-452; Steemers F J, Ferguson J A, Walt D. R. (2000) Screening Unlabeled DNA Targets with Randomly Ordered Fiber-Optic Gene Arrays, Nature Biotechnology, 18, 91-94; Ferguson, J. A., Steemers, F. J., Walt, D. R. (2000) High-Density Fiber-Optic DNA Random Microsphere Array, Analytical Chemistry, 72, 5618); radiofrequency encoding (see Nicolaou, K C, et al., Angew. Chem. Int. Ed. Engl. 34(20): 2289-2291,1995; Xiao, X, et al., Biotechnology and Bioengineering (Combinatorial Chemistry), 71(1): 44-50, 2000), or laser optical encoding (see Xiao, X., et al., Angew. Chem. Int. Ed. Engl., 36(7): 780-782, 1997).

Detecting can comprise sequencing DNA concatemers that are formed by ligating unique identifier nucleotide sequences together (Velculescu, et al., 1995, Serial analysis of gene expression, Science 270:484-487). To analyze unique identifier sequences using concatemer based sequencing detection, single stranded oligonucleotide ID tags or single stranded copies of replicated oligonucleotide ID tags need to be converted into double stranded DNA followed by restriction digestion to release the unique identifier sequences with staggered ends. The double stranded oligonucleotide ID tags or double stranded copies of replicated oligonucleotide ID tags can proceed directly to restriction digestion. Two restriction enzyme sites that flank the unique identifier nucleotide sequence of the oligonucleotide ID tag generate staggered ends when the enzyme acts on the oligonucleotide ID tag. These two restriction enzyme sites can be the same or different. Preferably, they select from restriction enzyme sites that generating greater than 4 overhanging nucleotides, for example the enzymes EcoRI and Bam HI, and the like. These unique identifier nucleotide sequences with staggered ends are ligated together randomly to form concatemers followed by inserting each concatemer into a plasmid vector for sequencing as described by SAGE technology (see Velculescu, et al., Science, 270:484-7, 1995). The frequency of each unique identifier nucleotide sequence appearing in the total sequence represents the relative abundance of the target. This method is very accurate for measuring both high abundant targets and low abundant targets since there is no bias towards high abundance targets.

The amount of each unique identifier nucleotide sequence can be quantified by various polymerase chain reaction methods performed in parallel. To date, there are many PCR-based methods for nucleic acid quantification including, for example, quantitative PCR (see Quantitative PCR Protocols, edited by Bemd Kochanowski and Udo Reischl, 1999, published by Humana Press), competitive PCR (see Ambion kits available from Austin, Tex.), 5′ nuclease PCR/TaqMan PCR (see Lie, et al., 1998, Current Opinion in Biotechnology, 9: 43-48) and Amplifluor PCR (see Uehara, et al, BioTechniques 26:552-558, 1999).

For example, there are several ways to design the oligonucleotide ID tags, which enable oligonucleotide ID tags to be analyzed with 5′ nuclease real-time PCR. For example, the oligonucleotide ID tag can be designed to contain a unique identifier nucleotide sequence, a common TaqMan™ fluorescence probe-annealing region and a universal PCR primer-annealing region. In order to analyze each unique identifier nucleotide sequence in parallel by TaqMan PCR, each PCR micro-tube is designed to specifically measure one unique identifier nucleotide sequence, in which a primer complementary to each unique identifier nucleotide sequence is used as a reverse primer for specifically priming the PCR reaction. The fluorescence released from the common TaqMan™ probe in each PCR microtube determines the amount of each unique identifier nucleotide sequence. In another example, in addition to unique identifier nucleotide sequences, another unique nucleotide sequence that is different from the unique identifier nucleotide sequence is designed into the oligonucleotide ID tag, together with the unique identifier nucleotide sequence to flank the common TaqMan™ probe. In this example, specific primers that are complementary to the unique identifier nucleotide sequence and the unique nucleotide sequence on one oligonucleotide ID tag are used to prime a specific PCR reaction in which fluorescence released from common TaqMan™ probe in each PCR micro-tube during PCR reaction determines the amount of each unique identifier nucleotide sequence. Using a unique nucleotide sequence together with a unique identifier nucleotide sequence to prime the quantitative PCR results in increased specificity for unique identifier nucleotide sequence detection. An oligonucleotide ID tag can also be designed to include one unique identifier nucleotide sequence flanked by two universal primer-annealing regions. In this example, the unique fluorescence TaqMan™ probe that specifically anneals with each unique identifier nucleotide sequence has to be used to quantify each unique identifier nucleotide sequence. Detailed methods can follow various TaqMan™ probe-based quantification of multiple DNA targets described elsewhere (de Baar et al., J. Clin Microbiol, 39(5): 1895-902, 2001).

Regular PCR without TaqMan™ probe can also be used to quantify a unique identifier nucleotide sequence. In order to obtain quantitative measurement of a PCR amplification product, PCR cycle number should be limited to the number that gives linear amplification of each oligonucleotide ID tag. Various methods can be used for quantification of an amplified oligonucleotide ID tag, for example gel electrophoresis followed by ethidium bromide staining or direct incorporation of a fluorescent nucleotide (see Hendrickson E R, et al. Nucleic Acid Research, 23:522-529, 1995) or dye such as ethidium bromide and SYBR Green (available from Molecular Probes, Eugene, O R).

PCR using an energy transfer primer, such as Amplifluor™ primer can also be used to quantify unique identifier nucleotide sequences (see Uehara, et al., BioTechniques 26:552-558, 1999). Amplifluor™ primer can be designed to anneal with a common sequence region or with the unique identifier nucleotide sequence. The specificity of detection is determined by primers complementary to the unique identifier nucleotide sequence and the quantity of a unique identifier nucleotide sequence is determined by fluorescence generated during the PCR reaction. Similar to analysis of a unique identifier nucleotide sequence by TaqMan™ real-time PCR, multiple real time PCR reactions can be set up in the same fashion to detect multiple unique identifier nucleotide sequence in different micro tubes. Equal amounts of sample containing oligonucleotide ID tags is added to the each tube. The quantity of each unique identifier nucleotide sequence is determined by the fluorescent signal emitted from the PCR reaction in each PCR micro-tube. Alternatively, Amplifluor™ primer can also be designed to specifically anneal with a unique identifier nucleotide sequence in which each PCR reaction in a micro-tube uses a unique identifier nucleotide sequence specific Amplifluor™ primer to specific quantify each unique identifier nucleotide sequence.

Other non-polymerase chain reaction nucleotide amplification methods can also be used to analyze unique identifier nucleotide sequences. For example, strand-displacement amplification or SDA (as described in Walker G T, et al. Nucleic Acids Res, 20:1691-1696 1992 and Proc Natl Acad Sci USA, 89: 392-396, 1992). ligase chain reaction or LCR (as described in Wu D Y, et al. Genomics, 4:560-569, 1989; Barany F, et al., Proc Natl Acad Sci USA, 88:189-193, 1991 and Birkenmeyer L G, et al., J. Virol Methods, 35:117-126, 1991), and Invader assay provided by Third Wave Technologies in Madison, Wis. (as described in Hessner M J et al, Clinical Chemistry 46:1051-1056, 2000). Based on the same principle used in tracking and detecting targets by a reporter ligand that is encoded with a oligonucleotide ID tag and binds specifically to the target, a competition assay can be designed to analyze targets in solution phase. The competition assay can analyze multiple targets in parallel.

Turning to the competition assay, an antagonist that can compete with a target is first encoded with a unique identifier nucleotide sequence by attaching an oligonucleotide ID tag to the antagonist. Antagonists comprise the same molecular substance as described in the targets above. Antagonists compete specifically with a target for the binding of the same receptor ligands. The antagonist can be the same molecule as the target, or a different molecule. For example, a natural protein can be an antagonist for the same natural protein in a test sample, or a recombinant protein synthesized in bacteria can be an antagonist of a natural protein. A synthetic peptide can be an antagonist of a natural protein. The antagonist can also be a small molecule or a small compound. In general the antagonist can be any molecule or binding entity that the target can be, for example, as listed herein. The oligonucleotide ID tag is attached to an antagonist to form a reporter antagonist. The procedures described for attaching oligonucleotide ID tags to ligands to form reporter ligands can also be applied when attaching oligonucleotide ID tags to antagonists to form reporter antagonists.

In the competition assay, reporter antagonists should be mixed with targets in a test sample first before adding receptor ligands into the test sample. The receptor ligands that can bind targets and reporter antagonists are the same substances described above for reporter-ligands. In order to promote competition between target and reporter antagonist in binding with receptor ligands, the amount of the receptor ligands added into the mixture containing both target and reporter antagonist should always be less than the combined amount of target and reporter antagonist added in the test sample in the mixture, thus, the targets have to compete with reporter antagonists to bind to receptor ligands. Only a fraction of targets and reporter antagonists can be collected through isolating the complexes that contains either reporter antagonists/receptor ligands complex or targets/receptor ligands complex.

Receptor ligands used in competition assay can be in solution phase or can also be in immobile phase. To separate the complexes from unbound free reporter antagonist, the same separation schemes described above for separation of the complexes containing reporter ligand-target can be applied. The separation methods can be designed to distinguish the complexes that contain reporter antagonist and receptor ligands from free reporter antagonist based on the difference of molecular mass. The methods that can be used for this purpose include but are not limited to electrophoresis, chromatography, centrifugation, filtration, etc. as described in above. The separation methods can also be designed to selectively isolate complexes by selective immobilization of the complexes to support surface, thus, unbound free reporter-antagonist can be washed off from support surface. The selective immobilization of the complexes can be through immobilizing receptor ligands. For example, when receptor ligands are antibodies, agarose conjugated protein A/G can selectively precipitate the complexes that contain reporter antagonists and receptor ligands by binding with the Fc regions of IgGs of receptor ligands. In another example, receptor ligands can be pre-immobilized on a support surface before contacting with the mixture containing both reporter antagonists and targets, and the free reporter antagonists can be washed off from the support surface.

The chemical environment described for promoting binding between reporter ligands and targets in the above can also be applied in the competition assay. The chemical environments also need to maintain the stringency that minimizes non-specific binding between reporter antagonists with receptor ligand. In this chemical environment, reporter antagonists bind specifically with their ligands to form complexes.

The oligonucleotide ID tags associated with the complexes that contain both reporter-antagonist and ligand can be dissociated, amplified, labeled or detected by the methods as described above for analyzing oligonucleotide ID tags.

In contrast to using reporter ligand to detect target in a test sample, in the competition assay, the signal intensity measurement from sequence-base detection is proportional to reciprocal of the concentration of target in the test sample. To calculate the absolute amount of each molecule target in the sample, two aliquots of a test sample are required to mix with reporter antagonist to form two mixtures, where each has a different ratio of reporter-antagonist to target, therefore, the absolute amount of a target in the test sample can be calculated if the concentration of reporter antagonist is known and the same amount of ligand is used for both aliquots of antagonist. For example, a test sample is subdivided to two aliquots, A and B. If we assume the concentration of target in the test sample is Xi, then in two aliquots of sample A and B, the amount C and amount RC of reporter antagonist can is added into aliquots A and B, forming the mixtures that have the ratio of target to reporter antagonist equal to Xi/C and Xi/RC, respectively. Here, R is a dilution factor between reporter antagonist added in aliquot A and aliquot B. In each mixture, the amount Y of the antibody that functions as a receptor-ligand can be added to mixture to bind to reporter antagonist or target in competition fashion. Because the amount Y of the antibody is less than the total of the reporter antagonist and the target contained in the mixture, only fraction of the reporter-antagonist binds with antibody. The amount of the reporter antagonist that binds with antibody is reciprocal to the target in the mixture; the higher concentration of target in mixture result in the lower amount of the reporter antagonist bind to receptor-ligand (which is an antibody in this example). The amount of the reporter antagonist that binds with the receptor-ligand can be measured by measuring the oligonucleotide ID tags associated with the complex of reporter antagonist-target. The oligonucleotide ID tags can be released from the complexes by the method described above for releasing oligonucleotide ID tags or their replicated copies from isolated complexes of reporter ligand-target, and the different sequence-based nucleotide detection methods as described above can be used for this purpose. The signal intensity, Si, generated from oligonucleotide ID tags isolated from aliquot A, should be proportional to YC/(Xi+C), or
Si=YC/(Xi+C),

same as the signal intensity from aliquot B, Si′, should be proportional to YRC/(Xi+RC), or
Si′=YRC/(Xi+RC).

The amount of the target in the test sample can then be calculated by the following formula:
Xi=RC(Si−Si)/(RSi−Si′),

wherein Xi is a concentration of target, C is a concentration of competitive reporter antagonist r that added into the first aliquot of the test sample, R is a dilution factor that is equal to the ratio of competitive reporter antagonist added into the first aliquot to that added into the second aliquot of the test sample, Si is a signal intensity of oligonucleotide ID tag (or its unique identifier nucleotide sequence) that derived from the first aliquot of the test sample in which the ratio of competitive reporter antagonist to its target is equal to C/Xi, and Si′ is a signal intensity of the oligonucleotide tag (or unique identifier nucleotide sequence) derived from the second aliquot of the test sample in which the ratio of competitive reporter to its target is equal to RC/Xi.

In the competition assay, different oligonucleotide ID tags can be used to encode different antagonists to form reporter antagonists, and a plurality of reporter antagonists can be used to compete with multiple targets for binding with receptor ligands, and all of oligonucleotide ID tags used for analyzing a test sample can be detected simultaneously, therefore, the concentration of multiple targets in a test sample can be calculated simultaneously using the same formula described above for every interested target.

To practice competition assay in this invention, the ratio of reporter-antagonists to target in the mixture can also be proportionally changed either by changing the concentration of reporter-antagonists added in different aliquots of sample, or by changing the concentration of target in different aliquots of sample, for example, the targets in different aliquots of sample can be diluted before adding the same amount of reporter antagonist into mixture, the dilution factor R is used to calculate the absolute amount of targets in the test sample The most important element for quantification is to form different ratio of reporter antagonist to target in two mixture. Three or more aliquots of a sample can be used to form mixtures with three or more ratios of reporter antagonist to target. The absolute amount of the target can be calculated by data regression. The data regression can be achieved with a computer data analysis program.

By the same logic, the competition assay can be designed to measure unbound free reporter antagonist instead of measuring reporter-antagonist bound with receptor ligand. Protein-DNA/RNA chimera molecule, prepared through in vitro translation or other methods, is of a protein fused with a fragment of DNA/RNA sequence that the protein is derived from. The protein DNA/RNA-chimera molecule can serve as a reporter-antagonist without needing an additional step to conjugate the oligonucleotide ID tag to protein. DNA/RNA fragment that fused with protein can serve as the unique identifier sequences. And the amount of DNA/RNA fragment is proportional to the protein molecule. There are several methods for deriving these kind protein-DNA/RNA chimera molecules for a population up to the whole genome proteins (as described in WO01/14539A2, WO 01/16352A1, WO 99/51773, Li M., Nature Biotechnology, 18:1251-1256, 2000; Hammond et al., J. Biol. Chem. 276:20898-20906, 2001; and Kurz M., et al., Nucleic Acid Research 28:e83, 2000). The DNA/RNA-protein chimera can be used as the reporter antagonists to measure the amount of a population of proteins including all the protein in a genome by competition assay. In addition, the competition assay can be performed in vivo. This can be carried out by transfecting a population (up to the total protein produced in a genome) of DNA/RNA-protein chimeras that are synthesized by in vitro translation, in which the DNA/RNA-protein Chimeras serve as reporter-antagonists. Two concentrations of reporter-antagonists are used to transfect the cells respectively by a protein transfection reagent such as Chariot by Active Motif (Carlsbad, Calif.). After entering cells, each reporter-antagonist will compete with endogenous naive protein for binding to their partner molecule inside the cells. Each endogenous protein and its reporter antagonist is also degraded at the same rate to reach its steady state concentration inside the cell. Then, proteins DNA/RNA chimera from both transfected cells are harvested separately, but using the same procedure to ensure the combined amount of each target and its corresponding DNA/RNA chimera is equal between two harvests and DNA/RNA sequences on protein DNA/RNA chimera can be used to identify and quantify the endogenous targets of transfected cells. These protein DNA/RNA chimera from each transfected cell population can be harvested by various methods as long as the combined amount of each target and its corresponding DNA/RNA chimera is equal among harvests to be compared. It includes isolating a cell organelle, such as nuclei, Golgi, endoreticulum, and cellular membrane and organelle membrane, and other biological compartments. Equal amount of the same biological compartment from different sample will ensure the equal amount of each target including endogenous target and its DNA/RNA chimera combined. The sequence based nucleotide acid detection methods described above can be used to detect DNA/RNA sequence that fused with protein chimera, therefore, the amount of each naive protein inside the cells can be simultaneously calculated using the competition method described above.

Turning to simultaneously detection for enzyme activities of multiple targets, with subject invention, the enzyme substrates are encoded with oligonucleotide ID tags to form reporter substrate. The reporter substrate can then be modified in a reaction catalyzed by the target enzymes, the enzymatic reaction to the reporter substrate can take place in both test tube or in living cells. The modified reporter substrates will be separated from unmodified reporter substrate, the oligonucleotide ID tags associated with the modified reporter substrates can be amplified, labeled, and simultaneously detected by the methods described above. The target enzyme is the same substance as the target described above and is capable of modification of a substrate in an enzymatic reaction. The target enzyme can be in solution phase or in immobilized phase. It can also be in living cells or in cell extract and in purified form. The substrate is a substance that the target enzyme can modify in an enzymatic reaction, e.g. peptide, protein, nucleic acid, carbohydratesetc. The substrate may be chemical synthesized, or enzymatic synthesized, it can also be naturally occurred or recombined. The enzymatic reaction can be any of naturally occurring post-translational modification and metabolic reaction, e.g. phosphorylation, acetylation, methylation, glycosylation, ubiquitination, etc.

The reporter substrate can be DNA/RNA-protein chimeras or substrate labeled with oligonucleotides as described above. When reporter substrate are proteins whose activity is determined by their post-translational modification. The reporter substrates can be used to detect post-translation modification of their corresponding endogenous protein/enzymes in vivo or in vitro. The reporter substrates are either transfected into cells in vitro or added to a cell free system in test tube. After a period of time when the anticipated post-translational modification is achieved, cell lysate is prepared. A capture ligand specific against the modified portion of the substrate is used to isolate the reporter substrate with the desired post-translational modification. The capture ligand is provided in excess to the number of modification moiety. Through analyzing the sequence of unique identifier sequence on reporter substrates, the identity and amount of post-translation modification on each reporter substrate can be obtained. If equal amount of reporter substrates are used for two separate samples, comparison on the level of post-translational modification of the same reporter substrate can be directly made. If the modification directly correlates protein activity and “on” or “off” of a signal transduction pathway. This method can effectively map out activation state of various signal transduction in a given biological system by using oligonucleotide-labeled regulatory proteins for various pathways as reporter antagonists. The proteins whose post-translational modification control “on” or “off” state of a signal transduction is described elsewhere (WO0127624 Shen et al. April 2001).

This invention applies to any enzyme whose substrate is known. Various enzymes can be found in “Methods in Enzymology” published by academic press and “Advances in Enzymology and Related Areas of Molecular Biology” published by Wiley, John & Sons and as described on the website of http://www.expasy.ch/cgi-bin/enzyme-search-cl including various oxidoreductases, transferases, hydrolases, glycosylase, lyases, ligases and isomerase.

In a preferable embodiment, the target enzymes are a group of cellular kinases in a living cell. A plurality of synthetic peptide substrates, each served as specific substrate for a target kinase, is conjugated with oligonucleotide ID tags to form reporter substrate. The reporter substrates will delivered into living cells by the chemical or physical means, such as by using transfection reagents, e.g. liposome, nuclear localization peptide, or physical methods used for transfection nucleic acid and protein. The cellular kinases catalyze phosphorylation of both endogenous substrates and transfected reporter substrates in the cell. Using a common phosphorylation-specific antibody, e.g. anti-phosphotyrosine, anti-phosphoserine or anti-phosphotheroine, the phosphorylated reporter substrates can selectively isolated from the cell extract. Anti-phospho antibodies, each specific for one substrate, can also be used to isolate enzyme modified reporter substrate. Many immunoprecipitation methods, which have been well established in the art, can be applied to isolate enzyme-modified substrates. e.g. commercial products offered by companies such as Cell Signaling (Beverly, Mass.) and Biosource (Camarillo, Calif.), Upstate (Waltham, Mass.), Santa Cruz (Santa Cruz, Calif.).

Applications of the subject invention can be practiced in a number of different ways. For example, micro-fluid based can be miniaturized onto labchips (Agilent Inc., Palo Alto, Calif.; Caliper Inc., Mountain View, Calif.) for simultaneous quantification of various targets in any given sample. As shown by various publications (Cohen, et al., Analytical Biochemistry, 273:89-97, 1999; Sundberg, et al., Current Opin in Biotech., 11:47-53, 1), labchip/microchip-based system not only provides an integrated system for a series of biochemical processes, but also consume minute amount of sample and deliver fast result. A labchip can be built with the following compartments:

A: sample,

B: reaction chamber,

C: reporter ligand,

D: PCR reagent mix,

E: oligo array with unique identifier nucleotide sequences,

F. washing buffer,

G: waste.

Sample containing targets to be detected are first sent from chamber A to B (reaction chamber) for immobilization. After washing, reporter ligands are sent from C to B for specific binding. Excess reporter ligand is washed away with buffer from chamber F. The PCR reagents containing fluorescent dye on a nucleotide in chamber D are sent to B to amplify unique identifier nucleotide sequences by PCR. After PCR, the solution in chamber B containing amplified unique identifier nucleotide sequences is sent out through a separation channel. The unique identifier nucleotide sequences are collected from a fixed position of the separation tube and sent to chamber E where oligo array is located. After hybridization, the amount of each unique identifier nucleotide sequence is determined based on fluorescent signal intensity of individual spot on the oligo array in Chamber E. Alternatively, both sample and reporter ligand can be sent out to chamber B for incubation followed by a separation step through a separation channel. Targeted molecule-bound reporter ligands are collected from a fixed position of the separation channel while sending free reporter ligands to waste chamber G. These ligands are then subjected to PCR in chamber D. After PCR, the solution in chamber D containing amplified unique identifier sequence is sent out through another separation channel. The unique identifier nucleotide sequences are collected from a fixed position of the separation tube and sent to chamber E where gene array is located. After hybridization, the amount of each unique identifier sequence is determined based on fluorescent signal intensity of individual spot on the oligo array in Chamber E.

Applications of the subject invention can also be used to create a database of biochemical data for a wide range of applications, including, for example, diagnosis of diseases states, the prognosis for recovery, determination of the onset (or potential therefore) of future disease states, assessment of health or medical condition and the like. The same practice except the method is followed according the PCT publication, WO01/20533 by Luminex titled as “Creation of A Database of Biochemical Data and Methods of Use”. In brief, multiple samples obtained from multiple subjects are subjected to quantitative profiling for a group of targets pre-selected using the methods described above and in U.S. Pat. No. 5,985,548. The multiple samples from multiple subjects include multiple samples from different individuals or multiple samples from the same individual on different time. The biological data generated is compiled electronically into a database along with each subject's phenotypic information and genetic information. After systematically collection a certain number of subjects, a statistical analysis is applied to extract one or more targets or a expression profile of a group of targets as the marker for the diagnosis of a disease, a disease states, the prognosis for a recovery, a future disease states and the assessment of a health or medical condition. The sample refers to any biological sample including, for example, body fluid and tissue biopsy.

Turning now to the figures, FIG. 1 depicts detection for soluble targets using molecular weight based separation scheme to separate reporter ligand-target complexes from free reporter ligands. The same oligonucleotide ID tag 10 is affixed to ligands that each is specific for epitope A and B of a target forming reporter ligand 12, 14. Reporter ligands 12,14 are then allowed to contact target 16 in contacting step 18 to form complex 20. Complex 20 is then separated from unbound reporter ligand 12, 14 by size exclusion chromatography, electrophoresis, or ultra-centrifugation in step 22. Step 24 detects and quantifies oligonucleotide ID tags using any suitable methods such as quantitative PCR, hybridization to a nucleic acid array or sequencing concatemers.

FIG. 2 depicts detection for soluble targets using selective immobilization scheme to separate reporter ligand-target complexes from unbound reporter ligands. Oligonucleotide ID tags a and b (30) are affixed with ligands (32) to form reporter ligand 33, 34 for target A and B respectively. Reporter ligands 33, 34 are then allowed to contact target A (36) and target B (37) to form complex 40 and 41 in solution phase. Once complexes are formed, a bead coated with a capture reagent 38 that is specific to a common epitope in both target A and target B is allowed to contact with the complexes 40 and 41 to form immobilized complex 44. Step 46 washes away free unbound reporter ligands from beads, and Step 48 amplifies and labels oligonucleotide ID tags that are associated with complexes and captured on the beads. Step 50 detects and quantifies oligonucleotide ID tags 30. Either quantitative PCR (step 52), hybridization to a nucleic acid array (step 54) or sequencing of the concatemer (step 56) is performed to detect and quantify the targets.

FIG. 3 depicts detection of specific immobilized targets. Oligonucleotide ID tags 60 and 62 are first labeled with moiety for detection 66 and then are affixed with ligands to form reporter-ligands 68, 70 for target A and B respectively. Targets A and B are selectively immobilized on a support surface by a capture ligand 64. The reporter ligands contact the targets on support surface in step 72, forming complexes bound on the support surface 74. Step 76 washes away unbound reporter ligands, and Step 78 dissociates the labeled oligonucleotide ID tags from support surface. Labeled oligonucleotide ID tags for targets A and B are represented in 80 and 82 respectively. The labeled oligonucleotide ID tags 80, 82 are detected simultaneously by hybridization in step 84 with a nucleic acid array, resulting in a signal on a nucleic acid array corresponding to 86 and 88 (for target A and B respectively) in step 90 comprising nucleic acid array detection.

FIG. 4 depicts detection of non-specific immobilized targets (e.g., fixed cell or tissue section, non-specific immobilized soluble cell lysate). Oligonucleotide ID tags 100 and 102, are affixed with ligands to form reporter ligand 106, 108 for target A and target B, respectively. Target A (110) and target B (112) are immobilized on the support surface 114, forming support surface with targets bound directly without capture molecules (e.g. a biopsy slide) 116. The slide 116 and reporter ligands 106 and 108 are contacted in step 118 to form immobilized reporter ligand-target complex 120. Step 122 washes away unbound reporter ligands, and Step 124 amplifies and labels the oligonucleotide ID tags associated with complex on the support surface. Step 126 detects and quantifies amplified copies of oligonucleotide ID tags including options 128 (quantification PCR), 130 (hybridization to an nucleic acid array) or 132 (sequencing concatemers).

FIG. 5 depicts detection of soluble targets in a competition assay. Targets A and B (144 and 146 respectively) are to be identified by oligonucleotide ID tags 142 and 140 (for target A and B respectively). Reporter antagonists 148 and 150 are formed by affixing oligonucleotide ID tags 142 and 140 to competitive antagonists for target A and B, respectively. Reporter antagonists for target A and B are mixed with targets 144 and 146, and contacted with receptor ligands 152 and 154 in step 156, forming complexes of receptor ligands bound to either targets or reporter antagonists for either A (158) or B (160). The reporter antagonist-receptor ligand and target-receptor ligand complexes are isolated in step 162 to obtain the complexes 158 or 160 in isolation. Finally, the oligonucleotide ID tags, 142 and 144 in the reporter antagonist-receptor ligand complexes, are analyzed simultaneously in step 164.

FIG. 6 depicts detection of targets on a cell surface. Double stranded DNA tags 165 are affixed with ligands to form reporter ligand 166, 167 for target A and target B respectively present on a cell surface 169. Sorting ligand 168 that is specific for a cell surface marker is labeled with a sorting moiety. Reporter ligands 166, 167 and sorting ligand 168 are allowed to contact with a mixed cell population at step 172 to form reporter ligand-target complex on the cell surface. Step 174 sorts out a cell population that contains the desired cell surface marker (173). Step 175 dissociates reporter ligands from sorted cell surface, and step 176 amplifies and labels DNA ID tags for detection.

FIG. 7 depicts detection of enzyme activity in a living cell. Oligonucleotide ID tag A and B are affixed with substrate A and B (177) to form reporter substrates 179, wherein the substrate A and B are specific substrates for the cellular enzymes A and B, respectively. Step 182 transfects the reporter substrate 179 into a living cell 181. The target enzymes in the environment of living cell modify the reporter substrates 183. Step 184 lyses cell and releases reporter substrate from living cells. Step 185 isolates enzyme modified reporter substrates, and oligonucleotide ID tags associated with modified reporter substrates are amplified and analyzed in step 186.

FIG. 8 depicts the general design principle of oligonucleotide ID Tag. In general, an oligonucleotide ID Tag can have different function regions, e.g., (I, II, III, IV) that are separated by inserts e.g., (A, B, C). In example 1, a unique identifier region (ID) is flanked by 2 universal regions (UP5, UP3). In example 2, two different ID regions are used (ID, ID′). The ID sequences are flanked by universal regions similar to example 1 (UP5, UP3). ID region is used to identify a oligonucleotide ID tag, the nucleotide sequence in ID region is different from each other among plurality of oligonucleotide ID tags used in an assay; other regions are used to facilitate amplification, labeling and/or detection of oligonucleotide ID tag. The nucleotide sequence in the regions other than ID region can share the same nucleotide sequence among plurality of oligonucleotide ID tags. ID region is the necessary component of oligonucleotide ID tag, other regions are considered as accessory regions. Depending on the desired method for oligonucleotide detection, different accessory regions can be included in oligonucleotide ID tags.

### C. EXAMPLES

### Example 1

### Simultaneous Analysis of Multiple Proteins in a Biopsy Section: Detection of p53, Epidermal Growth Factor (EGFR or erbB1), erbB2, erbB3, erbB4 and Estrogen Receptor (ER) in a Breast Cancer Biopsy

1. Sample Preparation

Fixed, paraffin-embedded breast cancer biopsy material is prepared using a protocol for conventional immunohistochemistry (http://www.gac.edu/cgi-bin/user/˜cellab/phpl?chpts/chpt2/intro2.html) prior to multiplex detection. Briefly, cancer tissue is fixed in Baker's Formalin Fixative, dehydrated with a series of increasing alcohol concentrations ranging from 30% to 100%, cleared sequentially in a 50:50 mixture of 100% ethanol: toluene, pure toluene, a 50:50 mixture of toluene and paraffin, and then embedded in paraffin. Sections (5-10 microns) of the embedded biopsy sample are made with a microtome, and immobilized on glass slides. Tissue slides are deparaffinized and hydrated by sequential incubation for 5 min in xylene, 100% ethanol, 95% ethanol, 70% ethanol and PBST (phosphate-buffered 0.9% NaCl containing 0.1% Tween 20). Slides are rinsed three times with PBST before use.

2. Conjugation of an Oligonucleotide ID Tag to an Antibody to form a Reporter Ligand.

An oligonucleotide ID tagged antibody (reporter-ligand) contains the general composition described in following:

5′ 3′
I A II B III C IV

Where,
I=Universal forward primer annealing region (UP5).
II=TaqMan quantitative PCR probe annealing region (TMP).
III=Unique identifier sequence region (ID).
V=Universal reverse primer annealing region (UP3).
A=Insert (or spacer) A
B=Insert (or spacer) B
C=Insert (or spacer) C

Each oligonucleotide ID tag contains an unique identifier sequence region or ID sequence, that serves as a unique identifier of the oligonucleotide ID tag. Each unique identifier nucleotide sequence is unique in nucleotide sequence, but all unique identifier nucleotide sequences retain similar melting temperatures. Other regions, such as UP5, UP3, TMP and Inserts A, B and C are accessory sequence regions and are identical in all oligonucleotide ID tags. These sequences are used to facilitate the amplification and detection of the oligonucleotide ID tag. The accessory regions are optional, depending on the analytical methods that will be employed for amplification and detection of the unique identifier nucleotide sequences.

In this application, TaqMan real-time quantitative polymerase chain reaction (PCR) will be used to analyze oligonucleotide ID tag composition, and therefore, the oligonucleotide ID tag ID is designed to contain the following:

Unique identifier nucleotide sequences (ID sequences) for each target:

| | | |
|---|---|---|
| EGFR: | 5′-ACGCTTAAGAAACCGCCTAC-3′; | (SEQ ID NO:1) |
| p53: | 5′-CACAGCACGGAAACAGGAGA-3′; | (SEQ ID NO:2) |
| erbB2: | 5′-ATATAGAACGCCCACTCGCA-3′; | (SEQ ID NO:3) |
| erbB3: | 5′-ATTATCCAAAAGCCCGACCG-3′; | (SEQ ID NO:4) |
| erbB4: | 5′-TATATATGCGCGTGCAAGCG-3′; | (SEQ ID NO:5) |
| ER: | 5′-AGCTTATTGTTTCGGGGTGC-3′; | (SEQ ID NO:6) |
| pg8: | 5′-ATTTTTGTGGCGGATCGCTG-3′; | (SEQ ID NO:7) |
| β-actin: | 5′-ACGTTTATGACGTGTTCGGC-3′. | (SEQ ID NO:8) |

Sequences for accessory regions:

| | |
|---|---|
| UP5 = | |
| 5′-TAGGCAGGAAGACAAACA-3′; | (SEQ ID NO:9) |
| UP3 = | |
| 5-ACAGCACCACAGACCA-3′; | (SEQ ID NO:10) |
| TMP = | |
| 5′-CTGGGCTCAACCCAGGAAGTG-3′ | (SEQ ID NO:11) |
| (bacterial nucleic acid sequence); | |
| Spacer A = | |
| 5′-AAGCTT-3′ | (SEQ ID NO:12) |
| (Hind III restriction site); | |
| Spacer B = | |
| 5′-GCGCGC-3′ | (SEQ ID NO:13) |
| (BssH II restriction site); | |
| Spacer C = | |
| 5′-CGGCCG-3′ | (SEQ ID NO:14) |
| (Eag I restriction site). | |

None of the unique identifier nucleotide sequences or sequences of UP3, UP5 and TMP share any sequence homology with human and mouse genes as assessed by a BLAST search.

In this example, a oligonucleotide ID tag has a structure of UP5-TMP-IDs-UP3, is a 78-mer oligonucleotide with the following sequence: 5′thiol/TAGGCAGGAAGACAAACA CTGGGCTCAACCCAGGAAGTG IDs TGGTCTGTGGTGCTGT-3′ (SEQ ID NO:15).

All 75-mer oligonucleotide ID tags share common accessory regions but contain different unique identifier nucleotide sequences. A thiol group is added to the 5′-end to facilitate antibody conjugation. Thiol-modified oligonucleotide ID tags are purchased from Integrated DNA Technologies, Inc. (Coralville, Iowa).

The following monoclonal antibodies (mAb) are purchased from commercial vendors: anti-p53, anti-EGFR, anti-erbB2 and anti-erbB3 are purchased from BD Transduction Laboratory (San Diego, Calif.); anti-erbB4 are from Santa Cruz Biotechnology, Inc. (Santa Cruz, Calif.); anti-ER is from Exalpha Biologicals, Inc. (Boston, Mass.); anti-phage coating protein VIII (pg8) is from Amersham Pharmacia (Piscataway, N.J.) and is used as the negative control, anti-actin is from Aldrich-Sigma Chemical Co. (St. Louis, Mo.) and is used as the positive control.

Each oligonucleotide ID tag is conjugated with a different antibody as described by Scheitzer (Scheitzer et al, 2000, Proc. Natl. Acad. Sci. USA, 97: 10113-10119) and Hendrickson (Hendrickson et al. 1995, Nucleic Acids Res., 23: 522-529). Briefly, each antibody is conjugated with a 5′-thiol-modified oligonucleotide ID tag using the crosslinking reagent, sulfo-GMBS (Pierce, Rockford, Ill.) at a molar ratio of 5:1. The antibody-oligonucleotide ID tag conjugate is purified by anion-exchange chromatography on Q-Sepharose (Amersham Pharmacia Biotech, Piscataway, N.J.) and size exclusion chromatography on Superdex-200 (Amersham Pharmacia Biotech, Piscataway, N.J.). The effect of conjugation on the ability of the antibody to bind antigen is determined via competitive ELISA assay as described by Ziporen (Ziporen et al., 1998, Blood, 92: 3250-9.).

Each oligonucleotide ID tag-conjugated antibody (reporter-ligand) is mixed to form a cocktail.

The amount of each reporter ligand is diluted to an equivalent immunoreactivity prior to preparing the cocktail.

3. Contacting the Reporter Ligand with Biopsy Slide and Isolating the Reporter Ligand-Target Complex

Tissue section slides prepared above are incubated for 30 min with normal mouse serum diluted 1:10 in PBST containing 10 μg/ml yeast tRNA to block non-specific binding. The slide is rinsed in PBST for 5 min and incubated for 60 min in a humidified chamber at 37° C. with the reporter-ligand cocktail diluted in PBST containing 10 μg/ml yeast tRNA and 1% normal mouse serum. The slide is washed three times in PBST for 5 min to remove unreacted reporter-ligand.

4. Detecting the Reporter Ligand by Real-Time PCR

Parallel TaqMan real-time quantitative PCR is used to quantify the unique identifier nucleotide sequences or ID sequence of the reporter ligands simultaneously, and is based on previous studies (Holland P M et al., 1991, PNAS, 88: 7276-7280; Lee L G et al., 1993, Nucleic Acids Res., 21: 3761-3766; Livak K J et al, 1995, PCR Methods Appl., 4:357-362.).

Reporter ligands captured on the tissue slide are released by two consecutive incubations of the slide with 100 μl of oligonucleotide ID tag dissociation buffer TagDB (50 mM Tris.Cl, pH 8.3, 100 μg/ml trypsin, 5 mM DTT and 0.2% Tween 20) for 10 min at 37° C. Two sequential incubations are pooled and added to a microtube, and protease inhibitor PMSF is added to a concentration of 1 mM. The solution containing the dissociated reporter-ligand is heated at 100° C. for 5 min to inactivate the trypsin. The solution is centrifuged briefly and the supernatant divided into 16 aliquots of 10 μl each in 16 microtubes. The unique identifier nucleotide sequence contained in the reporter ligand is quantified in duplicate by TaqMan real-time quantitative PCR. To each microtube is added the following reagents:

TABLE 1
Real-time TaqMan quantitative PCR reaction components

| | Volume/tube | Final | |
|---|---|---|---|
| Component | (μl) | Concentration | |
| Oligo-tag containing | 10.0 | — | |
| solution | | | |
| RNase-free water | 15.5 | — | |
| 10X TaqMan Buffer | 5.0 | 1x | |
| 25 mM MgCl₂ | 10.0 | 5.0 | mM |
| 2.5 mM dNTPs | 6.0 | 0.3 | mM |
| 10 μM UP5 5′primer | 1.0 | 0.2 | μM |
| 10 μM 3′primer | 1.0 | 0.2 | μM |
| 5 μM TMP probe | 1.0 | 0.1 | μM |
| AmpliTaq Gold DNA | 0.5 | 0.05 | U/μl |
| Polymerase (5.0 U/μl) | | | |

where UPS5 5′primer=5′-TAGGCAGGAAGACAAACA-3′ (SEQ ID NO:16) and TMP probe=5′FAM/CACTTCCTGGGTTGAGCCCAG/TAMRA-3′ (SEQ ID NO:17), where FAM and TAMRA are a fluorescence reporter dye and quenching dye, respectively. The UPS5′-primer serves as a universal PCR primer, and eight unique 3′ primers for the identifier nucleotide sequences to serve as specific PCR primers, where

EGFR unique identifier nucleotide sequence

(SEQ ID NO:18)

3′-primer = 5′-GTAGGCGGTTTCTTAAGCGT-3′;
p53 unique identifier nucleotide sequence

(SEQ ID NO:19)

3′-primer = 5′-TCTCCTGTTTCCGTGCTGTG-3′;
erbB2 unique identifier nucleotide sequence

(SEQ ID NO:20)

3′-primer = 5′-TGCGAGTGGGCGTTCTATAT-3′;
erbB3 unique identifier nucleotide sequence

(SEQ ID NO:21)

3′-primer = 5′-CGGTCGGGCTTTTGGATAAT-3′;
erbB4 unique identifier nucleotide sequence

(SEQ ID NO:22)

3′-primer = 5′-CGCTTGCACGCGCATATATA-3′;
ER unique identifier nucleotide sequence

(SEQ ID NO:23)

3′-primer = 5′-GCACCCCGAAACAATAAGCT-3′;
pg8 unique identifier nucleotide sequence

(SEQ ID NO:24)

3′-primer = 5′-CAGCGATCCGCCACAAAAAT-3′;
β-actin unique identifier nucleotide sequence

(SEQ ID NO:25)

3′-primer = 5′-GCCGAACACGTCATAAACGA-3′.

Each unique identifier nucleotide sequence 3′-primer is complementary to an unique identifier nucleotide sequence of the reporter ligand and is added to the PCR reaction to quantify the unique identifier nucleotide sequence. The quantity of each unique identifier nucleotide sequence from the dissociated reporter ligand represents the relative amount of antigen target in the slide.

Real-time quantitative PCR is carried out using an ABI PRISM 7700 Sequence Detection System under the following thermal cycling parameters: The initial cycle is HOLD at 50° C. for 2 min and 95° C. for 10 min, followed by 40 cycles each at 95° C. for 15 sec and 60° C. for 1 min. Data are analyzed using ABI PRISM 7700 SDS software.

A serial dilution of each reporter-ligand is used to calibrate TaqMan real-time quantitative PCR. The amount of reporter-ligand dissociated from a tissue section is calculated from a calibration curve, and the amount of target in the tissue slide is calculated based on the amount of corresponding reporter-ligand.

### Example 2

### Simultaneous Comparison of Tyrosine Phosphorylation Levels of EGFR, erbB2, erbB3 and erbB4 between Breast Cancer Cell Lines MCF-7 and MDA-MB-231

1. Sample Preparation

Cell lysates are prepared from ER-positive breast cancer cell line MCF-7 cells and ER-negative cell line MDA-MB-231 cells using a cell lysis buffer (CLB) containing: 50 mM Tris-HCl, pH 7.4, 150 mM NaCl, 1 mM DTT, 1 mM EGTA, 1 mM Na₃VO₄, 100 nM okadaic acid, 1 mM PMSF, 1 μg/ml each of aprotining, leupeptin and pepstatin, and 1% NP-40. Cells harvested from cell culture flasks are suspended in PBS and centrifuged for 5 min at 2,000 rpm/min to pellet the cells. Cell lysates are prepared on ice by mixing the cell pellets in CLB buffer on a vortex mixer, and collecting the supernatant by centrifugation. The protein concentration of the cell lysate is adjusted to 0.5 mg protein/ml.

2. Conjugation of an Oligonucleotide ID Tag to an Antibody to Form a Reporter Ligand.

Monoclonal antibodies (mAb) are purchased from commercial vendors: anti-p53, anti-EGFR, anti-erbB2 and anti-erbB3 are purchased from BD Transduction Laboratory (San Diego, Calif.); anti-erbB4 are from Santa Cruz Biotechnology, Inc. (Santa Cruz, Calif.); anti-ER is from Exalpha Biologicals, Inc. (Boston, Mass.). Fab fragments of these antibodies are prepared using ImmunoPure Fab Preparation Kit (Pierce, Rockford, Ill.). Fab fragments are conjugated with 5′-thiol-modified oligonucleotide ID tags using the same procedure described in Example 1. The ID sequence assigned to EGFR, p53, erbB2, erbB3, erbB4, pg8 and β-actin antigens are the same as described in Example 1. In this example, the oligonucleotide ID tags have a structure of UP5-IDs-UP3.

Oligonucleotide ID tag-conjugated Fab fragments are mix to form the reporter-ligand cocktail. Each reporter ligand is diluted to an equivalent immunoreactivity prior to preparing the cocktail.

3. Contacting Reporter Ligands with the Respective Antigen (Target) and Isolating the Reporter Ligand-Target Complexes

Add 100 μl of cell lysate prepared from either MCF-7 or MDA-MB-231 cells separately into 0.6 ml microcentrifuge tubes. To each microtube add 10 μl of cocktail of reporter ligand, and mix gently. Incubate the mixture at room temperature for 60 min to allow the oligonucleotide ID tag-conjugated antibodies to bind to their erbB receptor tyrosine kinase targets in the cell lysates. To selectively precipitate the tyrosine phosphorylated targets, 20 μl of anti-phosphotyrosine-agarose conjugate (Upstate Biotechnology, Lake Placid, N.Y.) is added to each microtube, which are incubated for 30 min at room temperature with constant mixing of the microtubes. The microtubes are centrifuged to pellet the agarose beads and the supernatant is discarded. The agarose beads are washed three times each with 200 μl PBST, and the beads are collected by centrifugation.

4. Detecting Tyrosine Phosphorylation in Four erbB Receptor Tyrosine Kinases from MCF-7 and MDA-MB-231 Using a Nucleic Acid Array

The oligonucleotide ID tag is released from the agarose beads by adding 50 μl oligonucleotide ID tag dissociation buffer TagDB to each tube and incubating for 10 min at 37° C. Protease inhibitor PMSF is added to each tube to a concentration of 1 mM, and the mixture containing the dissociated oligonucleotide ID tag is heated at 100° C. for 5 min to inactive the typsin. After a brief centrifugation, 10 μl of each supernatant corresponding to either the MCF-7 or MDA-MB-231 cell lysate are each transferred to a PCR microtube. The oligonucleotide ID tags are amplified and labeled in an asymmetric PCR reaction, where oligonucleotide ID tags bound with targets in MCF-7 cell lysate are labeled with the fluorescence dye Cy3 and bound with targets in MDA 231 cell lysate are labeled with fluorescence dye Cy5, respectively. The amplification and labeling are carried out using the following reagents:
10 μl 5×PCR buffer that contains 250 mM Tris-HCl, pH 8.3, 7.5 mM MgCl₂, 1 mM dCTP, dGTP DATP and dTTP
2.5 μl of 2 μM Cy3 or Cy 5 labeled UP5 primer, where Cy3UP5 primer for MCF-7 targets=5′-Cy3-TAGGCAGGAAGACAAACA-3′ (SEQ ID NO:26); and for Cy5UP5 primer for MDA-MD-231 targets=5′-Cy5-TAGGCAGGAAGACAAACA-3 (SEQ ID NO:27).
2.5 μl of 2 nM UP3 primer, where UP3 primer=5′-ACAGCACCACAGACCA-3′ (SEQ ID NO:28) and
2.5 unit Taq DNA polymerase
dH₂O to a final volume of 50 μl.
where 0.5 mM Cy3-dATP and Cy3-dUTP are added to PCR reaction containing oligonucleotide ID tags from the MCF-7 cell lysate, and 0.5 mM Cy5-dATP and Cy5-dUTP are added to the PCR reaction containing oligonucleotide ID tags from the MDA-231 cell lysate.

The asymmetric PCR reaction is carried out under the following thermal cycling parameters: The initial step is HOLD at 95° C. for 2 min followed by 20 cycles each at 94° C. for 1 min, 55° C. for 1 min and 72° C for 1 min, followed by one cycle at HOLD at 72° C. for 10 min. Equal amounts of PCR products from the two PCR reactions containing the Cy3-labeled oligonucleotide ID tags from MCF-7 cells and the Cy5-labeled oligonucleotide ID tags from MDA-MB-231 cells are combined into one tube.

A nucleic acid array for detecting unique identifier nucleotide sequences is prepared by non-contact printing on glass slides of the oligonucleotides complementary to the unique identifier nucleotide sequences or cIDs. 5′-amino-(CH₂)6-modified cIDs are synthesized by Integrated DNA Technologies Inc. (Coralville, Iowa) and the oligonucleotides are printed on silylated glass slides (TeleChem International, Inc., Sunnyvale, Calif.) using a micro dispenser (SuperArray, Inc., Bethesda, Md.). Before hybridizing with fluorescence-labeled oligonucleotide ID tags, the nucleic acid array slide is blocked by pre-hybridization with 0.5 ml GEAhyb Hybridization Solution (SuperArray, Inc., Bethesda, Md.) containing 50 μg/ml yeast tRNA. The prehybridization solution is removed and 50 μl of denatured and fluorescence-labeled oligonucleotide ID tags are mixed with 450 μl of GEAhyb Hybridization Solution and added to the nucleic acid array slide, which is incubated at 34° C. for 16 hr. After hybridization, the nucleic acid array slide is washed twice with 2×SSC containing 0.5% SDS at room temperature for 5 min, and twice with the same solution at 38° C. for 5 min. The washed slide is rinsed with 2×SSC and air dried. The hybridized array slide is scanned using a fluorescence laser scanner at an excitation wavelength of 532 nm and an emission wavelength of 635 nm (Axon Lab, Palo Alto, Calif.). Since only those reporter ligands bound to tyrosine phosphorylated targets are collected by precipitation and only oligonucleotide ID tags dissociated from the agarose beads are detected by the nucleic acid array, the intensity of the fluorescence signal hybridized to each spot of the nucleic acid array represents the amount of the tyrosine phosphorylated target in the cell lysate. Cy3- and Cy5-derived fluorescence represents the targets in MCF-7 and MDA-MB-231 cell lysates, respectively, and are detected simultaneously on each spot of the nucleic acid array. The relative change in tyrosine phosphorylation of the erbB receptor tyrosine kinases in MCF-7 cells and MDA-MB-231 cells is determined by comparing the fluorescence signal ratio of Cy3/Cy5.

### Example 3

### Simultaneous Analysis of Soluble Proteins in Serum: Profiling Angiogenic Factors aFGF, bFGF, Angiogenin, TGF-α and TGF-β

1.. Sample Preparation:

Blood is drawn from cancer patients to screen for levels of the angiogenic factors aFGF, bFGF, angiogenin, TGF-α and TGF-β. Cell-free serum is prepared by conventional protocols used in clinical laboratories (Mohan C, et al., 2001, Clinic Exp. Immunol., 123:119-26.), and 500 μl of serum from each patient is used for analysis.

2. Conjugation of an Oligonucleotide ID Tag to an Antibody to Form a Reporter-Ligand.

For each angiogenic factor, two monoclonal antibodies, each against a different epitope of the antigen, are conjugated with the same oligonucleotide ID tag to form reporter ligands. Monoclonal antibodies are obtained that specifically recognize aFGF (Upstate Biotechnology, Lake Placid, N.Y.), bFGF (Exalpha Biologicals Inc. Boston, Mass. and Chemicon International, Pittsburgh, Pa.), angiogenin (Biotrend Chemikalien GmbH and ACS Corp. Cologne, Germany), TGF-α (SeroTec Inc. Raleigh, N.C. and Santa Cruz Biotechnology, Santa Cruz, Calif.), TGF-β (Biosource International, Camarillo, Calif. and SeroTec Inc. Raleigh, N.C.) and the reference antigen pg8 (Amersham Pharmacia, Piscataway, N.J.). Two monoclonal antibodies that specifically recognize the same target are conjugated with the same oligonucleotide ID tags as described in Example 1.

The oligonucleotide ID tags have a structure of UP3-SPACER A-IDs-SPACER B-UP3 and the ID sequences assigned to each angiogenic factor are the following.

| | |
|---|---|
| aFGF: | |
| 5′-CATTACCCCTAAGGGATGC-3′; | (SEQ ID NO:29) |
| bFGF: | |
| 5′-AATTGCACAAGAGCCCACTC-3′; | (SEQ ID NO:30) |
| angiogenin: | |
| 5′-TACACGACTTTCGAGCGCAT-3′; | (SEQ ID NO:31) |
| TGF-α: | |
| 5′-AAGAAGCGACAACGGAGGAA-3′; | (SEQ ID NO:32) |
| TGF-β: | |
| 5′-ACTACACGTACACCGAGAGA-3′; | (SEQ ID NO:33) |
| pg8: | |
| 5′-ATTTTTGTGGCGGATCGCTG-3′. | (SEQ ID NO:34) |

Oligonucleotide ID tag conjugated antibodies are mixed to form the reporter ligand cocktail. Each reporter-ligand is diluted to an equivalent immunoreactivity prior to preparing the cocktail.

3. Contacting the Reporter Ligands with their Respective Targets in Serum and Isolating the Reporter Ligand-Target Complex.

To each 200 μl aliquot of serum sample is added 1 pM of the positive reference antigen pg8, and 10 μl of reporter ligand cocktail. Incubate the mixture at 37° C. for 60 min. The complexes formed between the reporter ligands and their respective targets in the serum each contain two reporter ligands bound to one angiogenic target. The molecule weight of these complexes is greater than 300 kDa, and is separated from free reporter ligand by gel filtration using a Sephacryl 500 column (Amersham Pharmacia Biotech Inc., Piscataway, N.J.). A sample of 200 μl of the reporter ligand-target reaction is applied to the column and eluted with Elution Buffer (150 mM NaCl, 50 mM sodium phosphate, pH 7.0 and 50 mg/ml BSA). Fractions containing the reporter ligand-target complex are combined and 50 μl of Protein A/G-agarose (Oncogene Science, Cambridge, Mass.) is added to precipitate the complexes. The antibody-antigen complexes captured by Protein A/G-agarose are collected by centrifugation. The agarose pellet is washed twice with

PBST and the agarose beads are collected by centrifugation.

4. Detection of Angiogenic Factors Using a Nucleic Acid Array.

To dissociate the oligonucleotide ID tag from the agarose pellet, mix 50 μl of oligonucleotide ID tag dissociation buffer TagDB with the pellet and incubate at 37° C. for 10 min. Protease inhibitor PMSF is added to the mixture to a concentration of 1 mM and the microtube is heated at 100° C. for 5 min to inactivate the trypsin. The microtube is centrifuged for 2 min and the dissociated oligonucleotide ID tags are recovered in the supernatant. The oligonucleotide ID tags are labeled with biotin by transferring 10 μl of supernatant to a microtube and adding the following reagents:
10 μl of 5×PCR buffer containing: 250 mM Tris-HCl, pH 8.3, 7.5 mM MgCl₂, 1 mM dCTP and dGTP, and 0.5 mM DATP and dTTP
5 μl of primer mix containing: 2 μM UP3 and UP5 primers, where UP3 primer=5′-ACAGCACCACAGACCA-3′ (SEQ ID NO:35) and UP5 primer=5′-TAGGCAGGAAGACAAACA-3′ (SEQ ID NO:36).
10 μl of biotin label (0.5 mM biotin-dUTP and 0.5 mM biotin-dATP)
2.5 units Taq DNA polymerase
dH₂O to a final reaction volume of 50 μl

The oligonucleotide ID tags are amplified and labeled with biotin by 20 cycles of PCR, and the PCR-amplified and biotin-labeled DNA is digested with 100 units each of BssH II and Eag I (New England Biolab, Beverly, Mass.) at 37° C. for 60 min followed by denaturation at 94° C. for 2 min and chilled on ice. The biotin-labeled and heat-denatured DNA is hybridized to a nucleic acid array slide prepared using the protocol described in Example 2. The biotin-labeled DNA hybridized to the nucleic acid array slide is detected by chemiluminescence after incubating the slide with streptavidin-conjugated alkaline phosphatase and a chemiluminescent substrate CDP-Star (Nonrad GEArray Detection Kit, SuperArray Inc., Bethesda). The chemiluminescent image is captured and analyzed using the FluorChem 8000 imaging system (Alpha Inotech, Oakland, Calif.).

The concentration of angiogenic factor in serum is calculated using the following equation:
Cxi=(Sxi)×(Cr/Sr)
where
Cxi is the concentration of angiogenic factor in serum,
Cr is the concentration of reference antigen added to the test sample, in this example it is 1 pM, Sxi is the chemiluminescent signal from the angiogenic factor detected in the array, Sr is the chemiluminescent signal from the reference antigen pg8 detected in the array.

### Example 4

### Simultaneous Quantification of Protein Targets in a Biological Sample by Competition Assay

1. Sample Preparation

A biological sample containing the targets of interest is solubilized with cell lysis buffer CLB described in Example 2, and the sample is adjusted to 0.5 mg protein/ml.

2. Conjugation of an Oligonucleotide ID Tag to a Peptide Competitor to Form a Reporter-Antagonist.

The reporter-antagonist has the same amino acid sequence as the peptide used to produce the target-specific antibody, and therefore, the reporter-antagonist competes with its target for binding to antibody. Antagonist peptides corresponding to different targets are synthesized by Research Genetics (Birmingham, Ala.), biotinylated with the EZ-Link Sulfo-NHS-LC-Biotinylation Kit (Pierce Chemicals, Rockford, Ill.). Biotinylated peptides are purified by D-Salt Polyacrylamide Desalting Columns ((Pierce Chemicals, Rockford, Ill.).

The oligonucleotide ID tag for the reporter-antagonist is of the same general composition as described in Example 1. In this application example, 100 32-mer unique identifier nucleotide sequences are generated by the method described by U.S. Pat. No. 5,654,413, in which each 32 mer oliognucleotide contains at least a 3 base difference in sequence and is not complementary to any of the other 32-mer oligonucleotides. In addition, all 32-mer oligonucleotides have a 50% G:C content and the same melting temperature. A 5′-biotinylated oligonucleotide ID tag synthesized by Integrated DNA Technologies Inc (Coralville, Iowa) is attached to the biotinylated reporter-antagonist using streptavidin.

Each streptavidin molecule has four biotin-binding domains, three biotinylated oligonucleotide ID tags and one biotinylated antagonist peptide are bound to one streptavidin molecule to form the reporter antagonist. To prepare the reporter antagonist, mix 300 μl of 1 μM biotin-oligonucleotide ID tag with 100 μl of 1 μM streptavidin in a microtube. In this mixture, three biotin-binding sites of streptavidin are occupied by three biotinylated oligonucleotide ID tags. Add 100 μl of 1 μM biotinylated reporter-antagonist to the microtube, and allow the mixture to react at room temperature for 30 min. Each reporter-antagonist will contain three oligonucleotide ID tags and one antagonist peptide per streptavidin molecule. The concentration of reporter-antagonist solution is adjusted to 100 nM. Reporter-antagonists for each target are prepared similarly.

A reporter-antagonist cocktail for 100 targets is prepared by mixing 10 μl of each reporter-antagonist (100 nM) in a microtube, and adjusting the final concentration of each reporter-antagonist to 1 nM. A peptide derived from pg8 is used as a reference control.

3. Contacting the Reporter-Antagonist to its Respective Target and Isolating the Reporter Antagonist-Target Complex

Antibodies against the peptide antigens are used as receptor-ligands in a competition assay. The immunoreactivity of each antibody is determined by ELISA assay using the synthetic antagonist peptide as antigen (Ziporen et al., 1998, Blood, 92:3250-9.). The immunoreactivity of each antibody with its respective antagonist peptide is determined, and the concentration of each antibody is adjusted to an equivalent immunoreactivity. To prepare a cocktail of 100 receptor-ligands for competition assay, the concentration of each antibody is adjusted to bind to 1 nM of antagonist peptide.

The cell lysate that contains the targets is diluted to a concentration of 0.5 mg protein/ml. An aliquots of 100 μl of each lysate is added to each of two microtubes labeled A and A′. Either 10 μl or 1 μl of the reporter antagonist cocktail is added to either microtube A or A′ to give respective final concentrations of 100 and 10 pM. After the reporter-antagonists are mixed thoroughly with the cell lysate, 1 μl of receptor ligand cocktail is added to each microtube, mixed on a vortex mixer and incubated at 37° C. for 60 min. After reaching equilibrium, the reporter-antagonists bound to the antibodies (receptor-ligand) are collected by the addition of 20 μl of Protein A/G-agarose (Oncogene Science, Cambridge, Mass.) and incubation at room temperature for 30 min on a shaker. The Protein A/G-agarose is recovered by centrifugation, and washed three times by centrifugation with 200 μl PBST.

4. Simultaneous Quantification of 100 Targets in a Biological Sample

The reporter-antagonists absorbed to Protein A/G-agarose are dissociated and amplified and labeled with biotin by a PCR reaction as described in Example 3. Biotin-labeled DNA is hybridized with a nucleic acid array slide containing 100 complementary nucleotides to the unique identifier nucleotide sequences of the oligonucleotide ID tags. The biotinylated DNA is detected by chemiluminescence, and the amplification, labeling and detection of the oligonucleotide ID tag is carried out using the same procedures described in

### Example 3

To calculate the absolute amount of each target molecule in the biological sample, the concentration of the target in the sample is Xi, C is the concentration of reporter-antagonist added to aliquot A (in this example it is 100 pM), R is the dilution factor of the reporter-antagonist added to aliquot A′ (in this example it is 1:10), and the concentration of reporter-antagonist added to aliquot A′ is equal to RC (in this example it is 0.1 C). Si is the chemiluminescent signal detected from the nucleic acid array that hybridized to the biotinylated DNA prepared from aliquot A, and Si′ is the chemiluminescent signal detected from DNA prepared from aliquot A′. Since the signal intensity of each spot on the nucleic acid array is proportional to the amount of oligonucleotide ID tag captured by Protein A/G-agarose, which is proportional to the ratio of reporter-antagonist to total molecules that can bind with the receptor-ligand, the signal intensity is equal to YC/(Xi+C) in aliquot A, and YRC/(Xi+RC) in aliquot A′, where Y is equal to the amount of antibody added to aliquots A and A′. Therefore, the signal intensity determined from the nucleic acid array hybridized with DNA from aliquot A and A′ is:

$$Si = Y \frac{C}{Xi + C}$$

$${Si}^{′} = Y \frac{RC}{Xi + RC}$$

In this example, Si and Si′ are calculated as:

$$Si = Y \frac{100 pM}{Xi + 100 pM} \qquad {Si}^{′} = Y \frac{10 pM}{Xi + 10 pM}$$

The formula for calculating the amount of target in the test sample (Xi) is:

$$Xi = RC \frac{{Si}^{′} - Si}{RSi - {Si}^{′}}$$

In this example, the target concentration is:

$$Xi = 10 pM \frac{{Si}^{′} - Si}{0.1 Si - {Si}^{′}}$$

Using the same equation, the chemiluminescence of different spots on the nucleic acid array can be used to calculate the concentration of 100 different targets simultaneously in the test sample.

### Example 5

### Simultaneous Monitoring of the Activation of Multiple Sisal Transduction Pathways

It is known that the activation of most, if not all, signal transduction pathways is associated with the phosphorylation of pathway-specific target proteins. Detecting changes in phosphorylation of these targets should therefore, be indicative of the relative activities of these pathways in the cell (See e.g., Table 2 below).

TABLE 2
Exemplary phosphorylation markers

| Pathway | Marker | Modification site |
|---|---|---|
| p44/42 ERK kinase | phosphoERK1/2 | Thr202/Tyr204 |
| pathway | | |
| p38 ERK kinase pathway | phosphop38 ERK | Thr180/Tyr182 |
| SAPK/JNK pathway | phosphoSAPK/JNK | Thr183/Tyr185 |
| NF6B pathway | phosphoI6B | Ser32/Ser36 |
| Wnt/insulin pathway | phosphoGSK-3 | Ser21/Ser9 |

1. Sample Preparation

To investigate the effect of serum stimulation on five signal transduction pathways in cultured NIH 3T3 cells, two flasks of NIH 3T3 cells are cultured to 50% confluence in DMEM containing 10% fetal bovine serum (Invitrogen, San Diego, Calif.). The medium is removed from the flask and the cells are washed three times with serum-free DMEM. One flask is incubated in serum-free DMEM and the second flask is incubated for 24 hr in DMEM containing 10% fetal bovine serum. Cells from each flask are harvested and cell lysates are prepared separately using cell lysis buffer CLB as described in Example 2. Each cell lysate is adjusted to 0.5 mg protein/ml.

2. Conjugation of an Oligonucleotide ID Tag to a Phospho-Specific Antibody to Form the Reporter-Ligand

Phospho-specific polyclonal antibodies are purchased from Cell Signaling Technology (Beverly, Mass.). The oligonucleotide ID tags have the same structure described in example 2 and the unique identifier nucleotide sequence assigned to each phospho-specific antibody is the following:

| | |
|---|---|
| ERK1/2 [phosphoThr202/Tyr204]: | |
| 5′-ATCTGAGCAAACGCAGCATG-3′; | (SEQ ID NO:37) |
| p38 MAPK[phosphoThr180/Tyr182]: | |
| 5ATTATCCAAAAGCCCGACCG-3′; | (SEQ ID NO:38) |
| SAPK/JNK[phosphoThr183/Tyr185]: | |
| 5′-TTTCCGACATCTGAGCCAAC-3′; | (SEQ ID NO:39) |
| I6B[phosphoSer32/Ser36]: | |
| 5′-CTAAACCCTCATAGGGACAC-3′; | (SEQ ID NO:40) |
| GSK-3 [phosphoSer21/Ser9]: | |
| 5′-TCATCACGACTACCGATGCA-3′; | (SEQ ID NO:41) |
| Pg8: | |
| 5′-ATTTTTGTGGCGGATCGCTG-3′. | (SEQ ID NO:42) |

The oligonucleotide ID tag is synthesized and conjugated to its respective antibody to form the reporter ligand by the procedure described in

### Example 1

### Each Reporter Ligand is Diluted to Give an Equivalent Immunoreactivity and Combined into a Single Cocktail

3. Contacting the Reporter-Ligands with Respective Targets and Isolating the Reporter Ligand-Target Complex

Polyclonal antibodies specific for ERK1, p38 MAPK, JNK, I6B, and GSK-3 are purchased from Santa Cruz Biotechnology (Santa Cruz, Calif.). These antibodies are used to selectively immobilize the target to a solid support. The capture antibody is first adsorbed to MaxiSorp microwell strips (Nunc, purchased from VWR Scientific Product, Chester, Pa.) following the manufacturer's protocol. The antibody-coated strips are incubated with PBST containing 10% normal rabbit serum for 30 min and washed three times with 200 μl PBST for 5 min. Cell lysates prepared from serum-depleted and serum-supplemented NIH 3T3 cell cultures are incubated with the antibody-coated strip at 100 μl of cell lysate (0.5 mg/ml) per well and incubated at 37° C. for 60 min to immobilize the target in the sample. The strips are washed three times with 200 μl PBST for 10 min, and blocked with 100 μl PBST containing 1% normal rabbit serum and 10 μg/ml yeast tRNA. The strips are then incubated with 100 μl PBST containing 10 μl of the oligonucleotide ID tag-conjugated antiphospho antibody mixture (reporter ligand) in 1% normal rabbit serum and 10 μg/ml yeast tRNA. The strips are incubated at 37 ° C. for 60 min to allow the reporter ligand to bind to its respective phosphorylated target. The strip is washed three times with 200 μl of PBST for 5 min to remove the free unbound reporter ligand.

4. Detecting Changes in Phosphorylation of Four Pathway-Specific Targets Using a Nucleic Acid Array

The oligonucleotide ID tags retained on the strips is amplified by PCR directly without their prior dissociation from the microwell. The same composition of reagents described in Example 2 is added to the strip. Cy3-UP5 primer is used to label the oligonucleotide ID tags from the serum-depleted cell lysate, and Cy5-UP5 primer is used to label the tags from the serum-supplemented cell lysate. Oligonucleotide ID tag amplification, labeling and detection are carried out as described in Example 2. The relative changes in phosphorylation of each target is determined by the ratio of Cy3/Cy5 fluorescence hybridized to each spot of the nucleic acid array.

### Example 6

### Simultaneous Analysis of Calmodulin-Binding Protein

1. Sample Preparation

Calmodulin (CaM) is involved in a number of cellular signaling pathways through direct protein-protein interactions. This assay is designed to quantify the levels of target CaM-binding proteins caldesmon, adducin, MARCK3, NAP22/CAP23, neuronal nitric oxide synthase (nNOS), metabotropic glutamate receptor 7A (mGluR7A), calpastatin, calpontin, neurogranin, twitchin kinase, titin kinase, and myosin light chain kinase in mouse brain tissue.

A cell lysate is prepared from neonatal mouse brain using cell lysis buffer CLB described in Example 1, and the cell lysate is adjusted to 0.5 mg protein/ml.

2. Conjugation of an Oligonucleotide ID Tag to an Antibody to Form a Reporter Ligand

Polyclonal antibodies specific for the CaM-binding proteins caldesmon, ryanodine, adducin, MARCK3, NAP22/CAP23, nNOS, mGluR7A, calpastatin, calpontin, neurogranin, twitchin kinase, CaM-dependent protein kinase, titin kinase and myosin light chain kinase are purchased from Santa Cruz Biotechnology (Santa Cruz, Calif.). Anti-pg8 is from Amersham Pharmacia (Piscataway, N.J.) and is used as a negative control. Each polyclonal antibody is conjugated to an oligonucleotide ID tag following the procedure in Example 1. The unique identifier nucleotide sequences assigned to each CaM-binding protein is the following:

| | |
|---|---|
| Caldesmon: | |
| 5′-GATTCACGTGATCCGATGC-3′; | (SEQ ID NO:43) |
| Adducin: | |
| 5′-TGATCCGATAGACGACTGCA-3′; | (SEQ ID NO:44) |
| MARCK3: | |
| 5′-AAACGCAGAAGAGGCACACA-3′; | (SEQ ID NO:45) |
| NAP22/CAP23: | |
| 5′-TTTGGAGCGTAAGCAGCATG-3′; | (SEQ ID NO:46) |
| nNOS: | |
| 5′-GTAAGCAGAGTAGGCAACAG-3′; | (SEQ ID NO:47) |
| mGluR7A: | |
| 5′-AGTAGGCAATGTACCCAGAC-3′; | (SEQ ID NO:48) |
| Calpastatin: | |
| 5′-AAAGACCCTCATGCAGCAGA-3′; | (SEQ ID NO:49) |
| Calpontin: | |
| 5′-AAGAGGCAACAAAGCGAAGG-3′; | (SEQ ID NO:50) |
| Neurogranin: | |
| 5′-ACTAAGCCTTTCGGAGTCTG-3′; | (SEQ ID NO:51) |
| Twitchin kinase: | |
| 5′-AACAAGGCTCATCCGATGTC-3′; | (SEQ ID NO:52) |
| Titin kinase: | |
| 5′-ACAAAGCCTCATCCGAAAGC-3′; | (SEQ ID NO:53) |
| Myosin light chain kinase: | |
| 5′-CATAAGGCAAACAGCGTTGC-3′; | (SEQ ID NO:54) |
| Pg8: | |
| 5′-ATTTTTGTGGCGGATCGCTG-3. | (SEQ ID NO:55) |

Each reporter-ligand is diluted to give an equivalent immunoreactivity and combined into a single cocktail.
3. Contacting Reporter Ligands with their Respective Target and Isolating the Reporter Ligand-Target Complex

CaM is purchased from Calbiochem (San Diego, Calif.). To selectively immobilize target proteins from the cell lysate, CaM is adsorbed to MaxiSorp strips following the manufacturer's protocol. The CaM-coated strip is blocked with 200 μl PBST containing 10% normal rabbit serum for 60 min followed by three washes with PBST. To each CaM-coated strip is added 98 μl of tissue lysate prepared as described above, and 2 μl of 50 mM CaCl₂. The strip is incubated at 4° C. overnight to allow the targets to bind. The strip is washed three times with 200 μl PBST containing 1 mM CaCl₂ for 10 min. The amount of target protein immobilized to the strip is measured by adding 100 μl of reporter ligand cocktail diluted 1:100 in PBST containing 1 mM CaCl₂ to each well. The strip is incubated at 37° C. for 60 min to binding the reporter-ligand to the immobilized target. The strip is washed three times with 200 μl PBST containing 1 mM CaCl₂ for 10 min. The reporter-ligand retained on the strip represents the amount of target in the lysate.

4. Detecting the Calmodulin-Binding Target Protein by Measuring the Amount of Oligonucleotide ID Tags

The oligonucleotide ID tag retained on the strip is amplified and labeled with biotin by PCR as described above. The amplification, labeling and detection procedures are described in Example 3. The amount of each calmodulin-binding protein in the tissue lysate is calculated based on the chemiluminescent signal detected in the hybridized nucleic acid array as described in Example 3.

### Example 7

### Simultaneous Detection of Multiple Cell Surface Antigens

This example illustrates the method of analyzing multiple cell surface targets using DNA ID-Tag in conjunction with flowcytometry cell sorting (FACS). In this example, anti-CD3 antibody was used to label spleen T lymphocytes for FACS sorting. DNA ID-Tag labeled antibodies were used to bind other cell surface antigens on the cell surface. Upon cell sorting, the DNA ID-Tag/antibodies immobilized on CD3+T cells were analyzed.

1. Sample Preparation

Mouse lymphocyte suspension was prepared from 8 weeks old BALB/c mouse spleen. Briefly, the spleen was crushed in a petridish in 5 ml 0.02 M Phosphate buffered saline (PBS) pH 7.4 using the back of a 10 ml disposable syring plug. The cell suspension was then filtered through a fine stainless steel sieve. The splenocyte was washed twice with FACS fluid (PBS, 0.2% Bovine serum albumin, 0.05% NaN₃). After centrifugation, the cell pellet was resuspended in FACS fluid at the density of 5×10⁶/ml.

2. Conjugation of an DNA ID-Tag to an Antibody to Form a Reporter Ligand Long DNA ID-Tag Preparation:

ID-Tag 1: ID tag 1 is a cDNA fragment of human bak gene (GenBank Acc. NM_{—}001188) position 288-512 (243 bp). Using PCR primer pair: Forward primer GAC ACA GAG GAG GTT TTC (SEQ ID NO:56) and reverse primer AGT ACT CAT AGG CAT TCT CT (SEQ ID NO:57), ID-tag 1 was amplified from human reference total RNA (CLONTECH) and cloned into pCR2.1 TOPO vector.

ID-tag 2: ID-tag 2 is a CDNA fragment of human TNFSF11 (TRANCE) (GenBank Acc. NM_{—}003701) position 463-714 (251 bp). Using PCR primer pair: forward primer ACT CTG GAG AGT CAA GAT AC (SEQ ID NO:58) and reverse primer AGA GGA CAG ACT CAC TTT AT (SEQ ID NO:59), ID-tag 2 was amplified from human reference total RNA (CLONTECH) and cloned into pCR2.1 TOPO vector.

Conjugation of ID tag to antibodies

Universal primer pair:

| | |
|---|---|
| Forward primer (TAF1): | |
| 5′-amino MC6/CGCCAGTGTGCTGGAATT | (SEQ ID NO:60) |
| (TAF1); | |
| and | |
| Reverse primer (TAR): | |
| CAGTGTGATGGATATCTGCA. | (SEQ ID NO:61) |

The universal primers anneal to the sequences on the pCR2.1 TOPO vector flanking the cDNA insert (ID-Tags). The 5′ amino-modified ID-Tags were prepared in a PCR reaction using the universal primer pair. The anti-mouse CD28 (Cat#553297, BD PharMingen, San Diego, Calif.) and anti-mouse CTLA4 (Cat#553719, BD PharMingen, San Diego, Calif.) were conjugated to ID-Tag 1 and 2 respectively using the published methodology described by Schweitzer (Schweitzer et al, 2000, PNAS, 97: 10113-10119) and Hendrickson (Hendrickson et al. 1995 Nucleic Acids Research 1995, 23: 522-529).
3. Contacting Reporter Ligands with their Respective Cell Surface Targets and Isolating the Reporter Ligand-Target Complex

5×10⁶ splenocyte was transferred into a 12×75 mm test tube. 50 μl anti-mouse Fc receptor antibody (Rat anti-mouse CD16/32, PharMingen Cat#553142, San Diego, Calif., 10 ug/ml in FACS fluid) was added to the cell suspension to block non-specific antibody binding to the lymphocytes. 10 μl of FITC conjugated anti-mouse CD3 (Cat#340960, BD Immunocytometry System, San Jose, Calif.) at 10 μg/ml, ID TagI conjugated anti-mouse CD28 at 10 μg/ml, and ID-Tag-2 conjugated anti-mouse CTLA4 at 10 μg/ml were added to the cell suspension. After mixing, the cell suspension was incubated on ice for 1 hr. It was then washed three times with 4 ml FACS fluid and resuspended in 1 ml FACS fluid. The cell suspension was filtered through a fine nylon screen to remove debris and then analyzed on a Becton Dickinson FACSCalibor cell sorter (Becton Dickinson Immunocytometry System, San Jose, Calif.). The cells positively stained with FITC-anti-CD3 antibody were sorted out and collected.

ID-Tag Retrieval and amplification: The 1×10⁵ sorted CD3+ lymphocytes were washed 3 time with PBS. After final centrifugation, the cell pellet was resuspended in 0.4 ml PBS. 50 μl freshly prepared Pronase (Cat#6911, Sigma, St. Louis, Mo.) solution (0.05% in PBS) was added to the cell suspension and incubated at 37° C. for 30 min. The mixture was then centrifuged at 1,500 rpm for 10 min. The supernatant was harvested and heated at 95° C. for 5 min to inactivate the pronase. The ID-Tags were precipitated by adding 0.1 volume of 0.1 M Ammonium acetate, pH5.5 and glycogen at a final concentration of 50 μg/ml and 1 volume of isopropanol. After incubating at −20° C. for 20 min, the precipitate was collected by centrifugation at 1,5000 rpm for 10 min. The DNA pellet was resuspended in 20 μl TE buffer.

4. Detecting the Cell Surface Proteins by Measuring the Amount of DNA ID Tags

DNA ID-Tag detection: 20 μl harvested DNA ID-Tags were amplified in a PCR reaction using with TAF1 and TAR primers in a 25 cycle PCR reaction as the following: The initial step is 2 min HOLD at 95° C. and followed by 25 cycles at 94° C. 1 min/55° C. 1 min/72° C. 1 min, followed by 72° C. HOLD for 10 min. The reaction mix was set up as the following: 50 mM Tris-HCl pH 8.5, 40 mM KCl, 2.5 mM MgCl₂, 8 mM Dithiotheitol, 0.2 mM of dATP/dCTP/dGTP, 0.05 mM dTTP, 0.1 mM Biotin-dUTP (Roche Biological).

Detection of DNA ID-Tags: The biotin labeled probe was detected by hybridization with cDNA array that contains bak and TRANCE cDNA fragment (HS-002 Apoptosis Q series GEArray, SuperArray Bioscience Corp. Frederick, Md.). The manufacture's hybridization and detection protocol was followed.

### REFERENCES

US Patent Documents
U.S. Pat. No. 4,683,195 July 1987 Mullis et al.
U.S. Pat. No. 4,683,202 July 1987 Mullis 435/91
U.S. Pat. No. 5,635,400 June 1997 Brenner et al. 435/320.1
U.S. Pat. No. 5,654,413 August 1997 Brenner et al. 536/22.1
U.S. Pat. No. 5,665,539 September 1997 Sano et al. 435/6
U.S. Pat. No. 5,985,548 November 1999 Collier et al. 435/6
U.S. Pat. No. 6,110,687 August 2000 Nilsen et al. 435/6
U.S. Pat. No. 6,264,825B1 July 2001 Blackburn et al. 205/777.5
U.S. Pat. No. 6,265,155B1 July 2001 Meade et al. 435/6
U.S. Pat. No. 6,268,149B1 July 2001 Meade et al. 435/6
U.S. Pat. No. 6,268,150B1 July 2001 Meade et al. 435/6

Other Publications
PCT/US99/08823 WO99/55886 Cen et al. Function-based gene discovery Nov. 4, 1999.
PCT/US00/25183 WO01/20533A2 Chandler et al. Creation of a database of biochemical data and methods of use March 2001.
WO0114589 Chandler M. Liquid array technology March 2001.
WO0127624 Shen et al. Compositions and methods for detecting protein modification and enzymatic activity April 2001.
PCT/US99/07203 WO 99/51773 Kuimelis et al. Addressable protein arrays October 1999.
PCT/US00/23414 WO01/16352A1 Kuimelis et al. Methods for encoding and sorting in vitro translated proteins Mar. 8, 2001.
PCT/US00/22906 WO01/14539A2 Min L. Methods and compositions for the construction and use of fusion libraries March 2001.
Chen, et al., 1986, Nuclease activity of 1,10-phenanthroline-copper: sequence-specific targeting, Proc. Natl. Acad. Sci. USA, 83:7147-7151.
Cohen, et al., 1999, A microchip-based enzyme assay for protein kinase A, Analytical. Biochemistry, 273:89-97.
Barany, et al., 1991, Genetic disease detection and DNA amplification using cloned thermostable ligase. Proc Natl Acad Sci USA 88:189-193.
Birkenmeyer, et al., 1991, DNA probe amplification methods, J. Virol Methods, 35:117-126.
Boidot-Forget, et al., 1988, Site-specific cleavage of single-stranded and double-stranded DNA sequences by oligodeoxyribonucleotides covalently linked to an intercalating agent and an EDTA-Fe chelate Gene 72(1-2):361-371.
Brown, et al., 1999, Exploring the new world of the genome with DNA microarrays, Nature Genetics Supplement 21:33-37.
de Baar, et al., One-tube real-time isothermal amplification assay to identify and distinguish human immunodeficiency virus type 1 subtypes A, B, and C and circulating recombinant forms AE and AG, J. Clin Microbiol, 2001, 39(5):1895-902.
Ferguson, et al., 2000 High-Density Fiber-Optic DNA Random Microsphere Array, Analytical Chemistry, 72:5618
Francois et al., 1988, Periodic cleavage of poly(dA) by oligothymidylates covalently linked to the 1,10-phenanthroline-copper complex, Biochemistry 27:2272.
Friedman et al., 1990, Molecular light switch for DNA: Ru(bpy)₂(dppz)²+, J. Am. Chem. Soc. 112:4960-4962.
Gordon R F and McDade R L. 1997. Multiplexed quantification of human IgG, IgA and IgM with Flowmetrix system. Clin. Chem. 43(9): 1799-1801.
Hammond, et al., 2001, In vitro selection and characterization of Bcl-X(L)-binding proteins from a mix of tissue-specific MRNA display libraries, 276:20898-20906.
Hendrickson, et al., 1995, High sensitivity multianalyte immunoassay using covalent DNA-labeled antibodies and polymerase chain reaction, Nucleic Acid Research, 1995, 23(3): 522-529.
Hessner, et al., 2000, Genotyping of factor V G1691A (Leiden) without the use of PCR by invasive cleavage of oligonucleotide probes, Clinical Chemistry 46:1051-1056.
Holland P M et al., 1991, Detection of specific polymerase chain reaction product by utilizing the 5′ - - - 3′ exonuclease activity of Thermus aquaticus DNA polymerase, Proc Natl Acad Sci U S A. 88: 7276-7280.
Kruse, N., Pette, M., Toyka, K., and Rieckmann, P. 1997. Quantification of cytokine MRNA expression by RT PCR in samples of previously frozen blood. J. Immunol. Methods 210(2):195-203.
Kurz, et al., 2000, Psoralen photo-crosslinked mRNA-puromycin conjugates: a novel template for the rapid and facile preparation of mRNA-protein fusions, Nucleic Acid Research 28:e83.
Lausch et al., 1995, Analysis of immunoglobulin G using a capillary electrophoretic affinity assay with protein A and laser-induced fluorescence detection, Electrophoresis 16:636-641.
Le Doan, et al., 1986, Targeted cleavage of polynucleotides by complementary oligonucleotides covalently linked to iron-porphyrins, Biochemistry 25:6736-6739.
Le Doan, et al., 1987, Sequence-specific recognition, photocrosslinking and cleavage of the DNA double helix by an oligo-[alpha]-thymidylate covalently linked to an azidoproflavine derivative, Nucleic Acid Res. 15:7749-7760.
Lee L G et al., 1993, Allelic discrimination by nick-translation PCR with fluorogenic probes, Nucleic Acids Res 21(16):3761-3766.
Lie, et al., 1998, Advances in quantitative PCR technology: 5′ nuclease assays, Current Opinion in Biotechnology 9:43-48.
Livak, et al. 1995, Oligonucleotides with fluorescent dyes at opposite ends provide a quenched probe system useful for detecting PCR product and nucleic acid hybridization, PCR Methods Appl. 4: 357-362.
Loeffler et. al. 2001, Nucleic acid sequence-based amplification of Aspergillus RNA in blodd samples, Journal of Clinical Microbiology, 39:1626-1629.
MacBeath, et al., 2000, Printing proteins as microarrays for high-throughput function deternination. Science 289: 1760-1763.
Min L., 2000, Applications of display technology in protein analysis, Nature Biotechnology, 18:1251-1256
Mohan, et al., 2001, Anti-subnucleosome reactivities in systemic lupus erythematosus (SLE) patients and their first-degree relatives. Clin Exp Immunol 123:119-26.
Nicolaou, et al., 1995, Radiofrequency encoded combinatorial chemistry, Angew. Chem. Int. Ed. Engi. 34(20):2289-2291
Okamoto, et al., 2000, Microarray fabrication with covalent attachment of DNA using bubble jet technology, Nature Biotechnology 18:438-441.
Ramsay G, 1998, DNA chips: state-of-the art, Nat Biotechnol. 16(1):404.
Ruzicka et al., 1993, Immuno-PCR with a commercially available avidin system, Science 260:698-699
Sano et al., 1992, Immuno-PCR: very sensitive antigen detection by means of specific antibody-DNA conjugates, Science 258, 120-122
Sano et al., 1991, A streptavidin-protein A chimera that allows one-step production of a variety of specific antibody conjugates, BioTechnology 9:1378
Scheitzer et al., 2000, Immunoassays with rolling circle DNA amplification: A versatile platform for ultra sensitive antigen detection, Proc. Natl. Acad. Sci. USA 97: 10113-10119.
Schweitzer, B., et al., 2000, Inaugural article: immunoassays with rolling circle DNA amplification: a versatile platform for ultrasensitive antigen detection, Proc. Natl. Acad. Sci. USA 97 (18):10113-10119.
Shoemaker et al., 1996, Quantitative phenotypic analysis of yeast deletion mutants using a highly parallel molecular bar-coding strategy, Nature Genetics 14 (4):450-465.
Sunberg S A, 2000, High-throughput and ultra-high-throughput screening: solution- and cell-based approaches, Current Opin in Biotech., 11:47-53
Steemers et al., 2000, Screening Unlabeled DNA Targets with Randomly Ordered Fiber-Optic Gene Arrays, Nature Biotechnology, 18:91-94
Strobel et al., 1990, Site-specific cleavage of a yeast chromosome by oligonucleotide-directed triple-helix formation, Science, 249:73-75.
Stults et al., 2001, Application of the 5′ Fluorogenic Exonuclease Assay (Real-time) for quantitative ribosomal DNA and rRNA analysis in sediments. Appl. Environmental Microbiol. 67: 2781-2789.
Tabor, et al., 1985, A bacteriophage T7 RNA polymerase/promoter system for controlled exclusive expression of specific genes, Proc. Natl. Acad. Sci. USA 82:1074-1078.
Zhao X et al., 2001, Immobilization of oligodeoxyribonuceotides with multiple anchors to microchips, Nucleic Acids Research 29: 955-959.
Ziporen et al., 1998, Defining an antigenic epitope on platelet factor 4 associated with heparin-induced thrombocytopenia, Blood 92(9): 3250-9.
Uehara, et al., 1999, Detection of telomerase activity utilizing energy transfer primers: comparison with gel- and ELISA-based detection, BioTechniques 26:552-558. Velculescu, et al., 1995, Serial analysis of gene expression, Science 270:484-487.
Walker, et al., 1992, Strand displacement amplification—an isothermal, in vitro DNA amplification technique Nucl Acids Res 1992, 20:1691-1696.
Walker, et al., 1992, Isothermal in vitro amplification of DNA by a restriction enzyme/DNA polymerase system, Proc Natl Acad Sci USA 89: 392-396.
Walt, D. R., 2000, Techview:Molecular Biology. Bead-Based Fiber-Optic Arrays, Science 287: 451-452.
Winn-Deen et al., 1993, Non-radioactive detection of Mycobacterium tuberculosis LCR products in a microliter plate format, Molecular and Cellular Probes 7:179-186.
Wu, et al., 1989, The ligation amplification reaction (LAR)—amplification of specific DNA sequences using sequential rounds of template-dependent ligation, Genomics 4:560-569.
Winzeler et al., 1999, Functional characterization of the S. cerevisiae genome by gene deletion and parallel analysis, Science 285:901-906.
Xiao, X. Y. and M. P. Nova in Combinatorial Chemistry; Synthesis and Application, eds.; S. R. Wilson, A. W. Czamik, Wiley, New York, 1996, chap.7
Xiao, X, et al., 2000, Solid phase combinatorial synthesis using mocrokan reactors, R f Tagging, and directed sorting, Biotechnology and Bioengineering (Combinatorial Chemistry), 71(1):44-50.
Xiao, X., et al., 1997, Combinatorial chemistry with laser optical encoding, Angew. Chem. Int. Ed. Engl. 36(7):780-782.
Zhou et al., 1993, Universal immuno-PCR for ultra-sensitive target protein detection, Nucleic Acid Research 21:6038-6039
Ziporen et al., 1998, Defining an antigenic epitope on platelet factor 4 associated with heparin-induced thrombocytopenia, Blood. 92(9): 3250-3259
Antibodies, A Laboratory Manual, 1988, by E d Harlow and David Lane, published by Cold Spring Harbor Laboratory.
Clinical Diagnosis and Management by Laboratory Methods, by Henry et al., 20^{th} edition, published by John Bemard Henry, W. B. Saunders Company.
Current Protocols in Molecular Biology, By Ausubel, et al., 2001, published by John Wiley & Sons, Inc.
Flurescent & Luminescent Probes: A practical Guide to Technology for Quantitative Real-Time Analysis by William T. Mason and W. T. Mason, published by Academic Press, Inc.
Handbook of Animal Lectins: Properties & Biomedical Applications, 2000, by David C. Kilpatrick, published by CRC Press.
Handbook of Capillary Electrophoresis, 1997, edited by James P. Landers, published by CRC Press.
Handbook of Plant Lectins Properties & Biomedical Applications, 1998, by E. J. Van Damme, published by Wiley, John & Sons, Inc.
High Resolution Chromatography, 1999, edited by P. A. Miller, published by Oxford University Press.
Nonradioactive labeling and detection of biomolecules, 1992, edited by C. Kessler, published by Springer-Verlag.
Quantitative PCR Protocols, 1999, edited by Bemd Kochanowski and Udo Reischl, published by Humana Press.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1-43. (canceled)

44. A method for assaying a plurality of different non-nucleic acid targets in a sample, which method comprises:
a) providing a plurality of target antagonists, each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags;
b) providing a plurality of receptor ligands, each receptor ligand specifically binding to a different target and its corresponding reporter antagonist in a competitive manner;
c) contacting a sample with said plurality of target antagonists and said plurality of receptor ligands provided in steps a) and b) under suitable conditions to allow competitive binding between said targets, if present in said sample, and their corresponding reporter antagonists, to their corresponding receptor ligands;
d) separating said target antagonists bound to said receptor ligands from said unbound target antagonists; and
e) assessing the identity and/or quantity of targets in said sample by detecting and/or quantifying said oligonucleotide ID tags in target antagonists bound to said receptor ligands.

45. The method of claim 44, wherein the sample is contacted with the plurality of target antagonists first and then contacted with the plurality of receptor ligands.

46. The method of claim 44, wherein the sample is contacted with the plurality of target antagonists and the plurality of receptor ligands simutaneously.

47. A composition for assaying a plurality of non-nucleic acid targets in a sample, which composition comprises a plurality of target antagonists, each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags.

48. The composition of claim 47, which further comprises a plurality of receptor ligands, each receptor ligand specifically binding to a different target and its corresponding reporter antagonist in a competitive manner;

49. A kit for assaying a plurality of non-nucleic acid targets in a sample, which kit comprises:
a) a composition of claim 47;
b) means for separating the target antagonists bound to the receptor ligands from the unbound target antagonists; and
c) means for detecting and/or quantifying said oligonucleotide ID tags in the target antagonists bound to the receptor ligands.

50. A composition, which composition comprises a plurality of complexes formed between a plurality of receptor ligands and their corresponding target antagonist, wherein each said receptor ligand specifically binds to a different target or its corresponding reporter antagonist in a competitive manner and each said target antagonist comprising a portion that specifically binds to a corresponding receptor ligand and an oligonucleotide identification (ID) tag, wherein said oligonucleotide ID tags in said target antagonists are distinguishable from each other based on an identifiable property other than the length of said oligonucleotide ID tags.

51. A method for assaying a plurality of different non-nucleic acid targets in a cell, which method comprises:
a) providing a plurality of target antagonists, each said target antagonist comprising a portion that specifically associates with a corresponding cellular component and an oligonucleotide identification (ID) tag, wherein said oligonucleotide ID tags in said target antagonists are distinguishable from each other based on an identifiable property other than the length of said oligonucleotide ID tags;
b) delivering said plurality of target antagonists into said cell to allow competitive interaction between said targets, if present in said cell, and said target antagonists, with said cellular components;
c) obtaining an equal amount of said cellular components associated with said targets or said target antagonists; and
d) assessing the identity and/or quantity of targets in said cell by detecting and/or quantifying said oligonucleotide ID tags in said target antagonists associated with said cellular components.

52. The method of claim 51, wherein the equal amount of the cellular component associated with the targets or target antagonists is obtained by isolating a biological structure from said cell.

53. A method for assaying activities of a plurality of enzymes in a sample, which method comprises:
a) providing a plurality of reporter substrates, each said reporter substrate comprising a portion that can be modified by a corresponding enzyme in a sample and an oligonucleotide identification (ID) tag, wherein said oligonucleotide ID tags in said reporter substrates are distinguishable from each other;
b) contacting said plurality of reporter substrates with said sample under suitable conditions to allow each enzyme to catalyze a modification reaction on its corresponding reporter substrate;
c) separating modified reporter substrates from unmodified reporter substrates; and
d) assessing the activities of said enzymes in said sample by detecting and/or quantifying said oligonucleotide ID tags in said modified reporter substrates.

54. The method of claim 53, wherein the oligonucleotide ID tags in the reporter substrates are distinguishable from each other based on an identifiable property other than the length of the oligonucleotide ID tags.

55. The method of claim 53, wherein the enzymes exist in vivo or in vitro.

56. The method of claim 53, wherein the enzymatic activity is assayed in situ.

57. The method of claim 53, wherein the modified reporter substrates are separated from the unmodified reporter substrates by contacting the reporter substrates with a capture reagent that specifically binds to the modification portion of the reporter substrates and that is immobilized on a surface.

58. A composition for assaying activities of a plurality of enzymes in a sample, which composition comprises a plurality of reporter substrates, each said reporter substrate comprising a portion that can be modified by a corresponding enzyme in a sample and an oligonucleotide identification (ID) tag, wherein said oligonucleotide ID tags in said reporter substrates are distinguishable from each other.

59. The composition of claim 58, wherein the oligonucleotide ID tags in the reporter substrates are distinguishable from each other based on an identifiable property other than the length of the oligonucleotide ID tags.

60. A kit for assaying activities of a plurality of enzymes in a sample, which kit comprises:
a) a composition of claim 59;
b) means for separating the modified reporter substrates from the unmodified reporter substrates; and
c) means for detecting and/or quantifying said oligonucleotide ID tags in the modified reporter substrates.

61. A composition, which composition comprises a plurality of reporter substrates, each said reporter substrate comprising a portion that has been modified by a corresponding enzyme in a sample and an oligonucleotide identification (ID) tag, wherein said oligonucleotide ID tags in said reporter substrates are distinguishable from each other.

62. The composition of claim 61, wherein the oligonucleotide ID tags in the reporter substrates are distinguishable from each other based on an identifiable property other than the length of the oligonucleotide ID tags.
